(19) United States
(12) Patent Application Publication
(10) Pub. No.: US 2010/0210713 A1
(43) Pub. Date: Aug. 19, 2010

(54) REGENERATION AND SURVIVAL OF CARDIAC PROGENITORS AND CARDIOMYOCYTES WITH A STRETCH ACTIVATED TRANSCRIPTION FACTOR

(75) Inventors: Kenneth R. Chien, Cambridge, MA (US); Yuh-Shin Chang, Boston, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/687,590

(22) Filed: Jan. 14, 2010

Related U.S. Application Data
(60) Provisional application No. 61/145,208, filed on Jan. 16, 2009.

(51) Int. Cl. A61K 48/00; C07K 14/00; A61P 9/10; A61P 9/00; A61K 31/7088

(52) U.S. Cl. 514/44 R; 530/350

(57) ABSTRACT

The present invention relates generally to the field of cardiovascular repair, and more particularly to methods and compositions for the survival of cells in response to mechanical stress and to the proliferation of postnatal cardiomyocytes. The present invention relates to a nuclear form of Creb312(N) polypeptide which when expressed in a cell promotes cell survival in response to stress such as mechanical stretch stress. Another aspect of the invention relates to the expression of the nuclear form of Creb312(N) polypeptide in a cardiomyocyte promotes propagation or regeneration of the cardiomyocyte. Other aspect relates to methods and compositions for gene therapy for the treatment of cardiac diseases and disorders in a subject comprising increasing the expression of Creb312(N) polypeptide in cells in vivo or ex vivo, such as cardiac cells and cardiac progenitor cells, and in some embodiments, subsequent transplantation of the Creb312 expressing cells into a subject for the treatment of cardiac diseases and disorders in the subject.

### CROSS REFERENCED APPLICATIONS

This application claims the benefit under 35 U.S.C. 119(e) of U.S. Provisional Application Ser. No. 61/145,208 filed on Jan. 16, 2009, the contents of which is incorporated herein in its entirety by reference.

### FIELD OF THE INVENTION

The present invention relates generally to the field of cardiovascular repair, and more particularly to methods and compositions for the proliferation of postnatal cardiomyocytes.

### BACKGROUND

Cardiovascular disease involves diseases or disorders associated with the cardiovascular system. Such disease and disorders include those of the pericardium, heart valves, myocardium, blood vessels, and veins.

Over the last two decades, the morbidity and mortality of heart failure has markedly increased (Tavazzi, 1998). Therefore, finding an effective therapeutic method is one of the greatest challenges in public health for this century. Although there are several alternative ways for treatment of heart failure, such as coronary artery bypass grafting and whole-heart transplantation, myocardial fibrosis and organ shortage, along with strict eligibility criteria, mandate the search for new approaches to treat the disease.

Cell transplantation has emerged to be able to increase the number of contractile myocytes in damaged hearts. However, cardiomyocytes, which are also known as cardiac muscle cells, are terminally differentiated cells and are unable to divide and their use in cell transplantation is limited by the inability to obtain sufficient quantities of cardiomyocytes for the repair of large areas of infarct myocardium. Thus, alternative sources of cells for cell transplantation need to be used, such as stem cells. However, the use of using non-committed stem cells also possess the risk of their differentiation into non-cardiac cells and risk of teratomas post transplantation. Furthermore, use of most cell types in a cell-based therapy approach is limited due to the mechanical stress placed on the transplanted cells once they are implanted into the heart, i.e. the mechanical forces due to the contraction of the heart muscle wall. Thus, there is a need in the art to develop alternatives to the presently used cells and transplantation techniques for the treatment of cardiovascular disease.

Myocardial infarction (MI) is characterized by the death of myocytes, coagulative necrosis, myocytolysis, contraction band necrosis, or apoptosis, resulting from a critical imbalance between the oxygen supply and demand of the myocardium. The most common cause of MI is coronary artery thrombosis following the rupture of atheromatous plaques. Though once strictly defined as a lack of blood flow, the modern definition of ischemia emphasizes the imbalance between oxygen supply and demand as well as the inadequate removal of metabolic waste products. Impaired oxygen delivery results in a reduction in oxidative phosphorylation that causes anaerobic glycolysis. This produces excess lactate that accumulates in the myocardium. Impaired ATP production and acidosis results in a decline in myocardial contractility. Similarly, ischemia reperfusion injury, without total occlusion, can also cause cardiac damage The exposure of the contents of the plaque to the basement membrane following plaque rupture ultimately results in vessel blockage culminating from a series of events including platelet aggregation, thrombus formation, fibrin accumulation, and vasospasm. Total occlusion of the vessel for more than 4-6 hours results in irreversible myocardial necrosis. Ultimately, death and morbidity from myocardial infarction is the result of fatal dysrhythmia or progressive heart failure. Progressive heart failure is chiefly the result of insufficient muscle mass (deficiency in muscle cells) or improper function of the heart muscle, which can be caused by various conditions including, but not limited to, hypertension. Progressive heart failure is, therefore, the focus of cell-based therapy.

All current strategies for the treatment of myocardial infarction focus on limiting myocyte death. Annually in the United States, 500,000 patients undergo angioplasty with stent placement. 400,000 will undergo coronary artery bypass, while an unknown additional number of patients will be treated by thrombolytic therapy.

One approach, known as cellular cardiomyoplasty, has received recent attention and focuses on repopulation and engraftment of the injured myocardium by transplantation of healthy cells (Reffelmann, T. and Kloner, R. A. (2003) Cardiovasc Res. 58 (2): 358-68), including transplantation of stem cell populations and cardiac progenitor cells. Many cell types that might replace necrotic tissue and minimize regional scarring have been considered. Cells that have already committed to a specific lineage, such as satellite cells, cardiomyocytes, primary myocardial cell cultures, fibroblasts, and skeletal myoblasts, have been readily used in cellular cardiomyoplasty with limited success in restoring damaged tissue and improving cardiac function (Menasche, P. (2003) Cardiovasc Res. 58 (2): 351-7; Etzion, S. et al (2001) J Mol Cell Cardiol. 33 (7): 1321-30; Sakai, T. et al (1999) L Thorac. Cardiovasc. Surg. 118 (4): 715-24).

Cardiogenic progenitors (also known as cardiac progenitor cells) are precursor cells that have committed to the cardiac lineage, but have not differentiated into cardiac muscle. Cardiomyocytes are the cells that comprise the heart. They are also known as cardiac muscle cells. Use of cardiomyocytes in the repair of cardiac tissue has been proposed. However, this approach is hindered by an inability to obtain sufficient quantities of cardiomyocytes for the repair of large areas of infarcted myocardium. There are also problems regarding the incorporation and tissue-specific function of intra-cardiac grafts derived from cardiomyocytes, even when they are harvested from embryonic sources (Etzion, S. et al (2001) J. Mol. Cell. Cardiol. 33 (7): 1321-30). Intra-cardiac grafts using this cell type can be successfully grafted and are able to survive in the myocardium after permanent coronary artery occlusion and extensive infarction. However, engrafted rat embryonic cardiomyocytes attenuate, but do not fully reverse, left ventricular dilatation and prevent wall thinning. While survival was improved during 8 weeks of follow-up, the implanted cells did not develop into fully differentiated myocardium. Surprisingly, they remained isolated from the host myocardium by scar tissue and did not improve systolic function over time (Etzion, S. et al (2001) J. Mol. Cell. Cardiol. 33 (7): 1321-30).

Congestive heart failure (CHF) is a clinical condition in which a primary or secondary circulatory system disease causes abnormal cardiac pressure or performance characteristics that lead to pulmonary congestion (Zhang, J. and Narula, J. (2004) Surg Clin N Am 84:223-242). CHF is a chronic condition that results when the heart muscle is unable to pump blood as efficiently as is needed to maintain physiological homeostasis. Although common causes of CHF include hypertension, anemia and cardiomyopathy, CHF is most often caused by myocardial infarction (Lee, Michael, et al (2004) Reviews in Cardiovascular Medicine, 5: 82-94). Currently, nearly 550,000 new cases of heart failure are now diagnosed each year.

CHF occurs when cardiac dysfunction prevents adequate perfusion of peripheral tissues. Inadequate perfusion leads to stimulation of compensatory mechanisms that then cause many of the clinical signs and symptoms of the condition. In patients with CHF, neurohumoral compensatory mechanisms are activated. Chronic stimulation by these agents increases cardiac afterload and preload, further worsening ventricular function.

Small molecule therapeutics are being tested with the aim to disrupt these compensatory systems. However, no pharmacological intervention improves the underlying pathophysiology of CHF.

Surgical intervention to treat CHF is also limited. Cardiac transplantation is the mainstay of treatment for patients with end-stage cardiomyopathies, such as CHF, but is limited by the scarcity of donor organs and complications, such as graft rejection and allograft coronary vasculopathy (Fedak, P. et al (2003) Seminars in Thoracic and Cardiovascular Surgery, 15: 277-286).

In summary, loss of function and/or cell mass in cardiac muscle can arise, for example, by physical damage or disease-related damage (e.g., genetic or acquired disease). Stem cell technology has made cellular myoplasty a realistic treatment for restoring or enhancing cardiac muscle function or cell mass. Tissue-specific stem cells and embryonic stem cells provide limited results.

### SUMMARY

One aspect of the present invention relates to methods and compositions to promote the survival of a cell or a population of cells in response to stress, such as mechanical stretch-induced stress. Another aspect of the present invention also provides compositions and methods for increasing proliferation, increasing cell cycle activity, and/or proliferation of postmitotic mammalian differentiated cardiomyocytes. In addition, other aspects of the present invention relate to methods and compositions discussed herein for the use to promote survival of cells in response to cellular stress, such as mechanical-induced stress. Another aspect relates to methods and compositions described herein to promote survival of cells exposed to such stress following transplantation or grafting into a subject. In one aspect of the present invention, the methods and compositions can be used to slow, reduce, or prevent the onset of cardiac damage caused by, for example, myocardial ischemia, hypoxia, stroke, or myocardial infarction in vivo.

In particular, one aspect of the present invention relates to methods and compositions for increasing survival of a cell to stress, such as mechanical-induced stress by increasing the level of a nuclear form of Creb312 polypeptide (Creb312(N)) in a cell that is at risk of, or has been exposed to cellular stress, such as mechanical stretch-induced stress. In some embodiments, the compositions and methods can comprise at least one agent which results in an increase in the level of a nuclear form of Creb312 polypeptide (Creb312(N)) in a cell, such as an agent which increases the proteolytic cleavage of full length Creb312 to the truncated Creb312(N), wherein the nuclear form of Creb312 (Creb312(N)) polypeptide comprises amino acids 1-376 of SEQ ID NO: 2. In some embodiments, an agent is a polypeptide of SEQ ID NO: 3 or a homologue, or functional variant or functional fragment thereof.

Alternatively, in some embodiments of this aspect and all other aspects described herein, the compositions and methods for increasing the survival of a cell to stress such as mechanical stress comprises contacting the cells with at least one agent which is a nucleic acid encoding a Creb312(N) polypeptide of SEQ ID NO: 3 or a functional fragment or functional variant thereof, where the nucleic acid encoding a promoter is operatively linked to a promoter for expression of the Creb312(N) polypeptide in the cell.

Another aspect of the present invention provides compositions and methods for increasing proliferation, increasing cell cycle activity, and/or proliferation of postmitotic mammalian differentiated cardiomyocytes, in some embodiments, the methods and compositions as discussed herein can be used to slow, reduce, or prevent the onset of cardiac damage caused by, for example, myocardial ischemia, hypoxia, stroke, Or myocardial infarction in vivo. In addition, the methods and compositions as discussed herein can be used to enhance proliferation of differentiated cardiomyocytes in vitro and/or ex vivo, which can then be used in tissue grafting.

The invention is based, in part, on the discovery that the full length Creb312 polypeptide, which is an ER membrane bound polypeptide which is a regulated intramembrane proteolysis (RIP) protein, and is cleaved into truncated active nuclear Creb312 form (herein referred to “Creb312(N)”) which translocates to the nucleus and functions as a transcription factor to initiate gene transcription of some cell survival genes in response to cellular stress. In particular, the inventors have discovered that full length Creb312 is developmentally expressed in cardiac tissues, such as the primary and secondary heart fields, and have discovered, using Creb312 null (−/−) mutant mice that loss of Creb312 results in significant (i.e. as much as 50-100%) increase in apoptotic cell death in the heart at a time when the heart begins beating. The inventors have also demonstrated that Creb312 provides a cytoprotective role following myocardial infarction or ischemia. The inventors also discovered that full length Creb312 polypeptide is proteolytically cleaved to the active, nuclear form of Creb312 (Creb312(N)) by chemical, mechanical, ER-stress, hypoxia and hormones, and that this activation (i.e. cleavage of the fill length Creb312 to the nuclear active Creb312 form) is mediated via the ILK and Akt/PKB pathways. The inventors have further discovered that overexpression of Creb312 results in less apoptotic death in response to stress, such as mechanical-induced stretch, and that the Creb312(N) polypeptide, by itself or as a homodimer functions as a cell survival factor in response to mechanical stress and also promotes cardiomyocyte proliferation and myocardial regeneration.

Some aspects of the invention is based, in part, on the discovery that the nuclear form of Creb312 (herein referred to “Creb312(N)” (which is a truncated polypeptide of the full length Creb312 polypeptide), and fragments of Creb312(N) promote cardiomyocyte proliferation and myocardial regeneration. Without wishing to be bound to theory, the adult mammalian heart responds to injury with scar formation, not with proliferation as the cellular basis for regeneration. The insufficient regeneration of mammalian hearts is explained by the contractile apparatus impinging on cardiomyocyte division. The inventors have demonstrated herein that a Creb312(N) polypeptide can induce cell cycle re-entry of differentiated mammalian cardiomyocytes. In particular, the inventors have demonstrated that a Creb312(N) polypeptide can stimulate mononuclear cardiomyocytes, present in the adult mammalian heart, to undergo the full mitotic cell cycle. The proteolytic cleavage of a full length Creb312 polypeptide to a truncated Creb312(N) polypeptide induces cardiomyocyte, proliferation, Cleavage of a full length Creb312 polypeptide to Creb312(N) occurs by the activation of the ILK and Akt/PI3K signaling pathways. After myocardial infarction, truncated Creb312(N) polypeptide induces cardiomyocyte cell cycle re-entry, improves cardiac remodeling and function, reduces fibrosis and infarct size, and increases angiogenesis. These results demonstrate that a truncated Creb312(N) polypeptide regulates is a new target for innovative strategies to treat heart failure.

The methods and compositions as discussed herein encompass any means to increase the level of the nuclear form of Creb312 in a cell and contacting the cell with an agent or entity which increases the level of the nuclear form of Creb312 within the cell. In some embodiments, agents useful in all aspects disclosed herein include, but without limitation, small molecules, nucleic acids, nucleic acid analogues, proteins, protein fragments, aptamers, antibodies etc as discussed herein in more detail.

As discussed above, another aspect of the present invention relates the methods and compositions for inducing the proliferation of post-mitotic or differentiated cardiomyocytes. In some embodiments of this aspect and all other aspects disclosed herein, a cardiomyocytes is, for example a postnatal cardiomyocyte, an adult cardiomyocytes or cardiomyocyte progenitor. In some embodiments of this aspect and all other aspects disclosed herein, the present invention relates to methods and compositions to increase the level of the nuclear form of Creb312 polypeptide, such as amino acids 1-376 of SEQ ID NO: 2 or to increase the level of the protein corresponding to SEQ ID NO: 3 or a homologue, or functional variant or functional fragment thereof in a cardiomyocyte. Any means to increase the level of a nuclear form of Creb312 (i.e. a Creb312(N)) polypeptide in a cardiomyocyte can be used in the methods as disclosed herein, including but not limited to, contacting a cardiomyocyte with an agent or entity which increases the level of a Creb312(N) polypeptide within the cell. In some embodiments of all aspects disclosed herein, useful agents include, but without limitation, small molecules, nucleic acids, nucleic acid analogues, proteins, protein fragments, aptamers, antibodies etc as discussed herein in more detail.

In some embodiments, an agent comprises a nucleic acid which encodes a polypeptide which has at least 80% sequence identity to SEQ ID NO: 3, or alternatively at least 85%, or at least 90%, or at least 95%, or at least 98%, or at least 99%, or at least 100% sequence identity to SEQ ID NO: 3. In some embodiments, an agent is a protease, such as a site-1 protease (SIP) and/or a site-2 protease (S2P), which is capable of cleaving full length Creb312 polypeptide (SEQ ID NO: 2) into the nuclear (active) form of Creb312 polypeptide (i.e. residues 1-376 of SEQ ID NO: 2). In alternative embodiments, an agent is any agent which activates the PI3K/Akt (PKB) pathway.

In some embodiments, an agent which increase the level of the nuclear form of Creb312 (Creb312(N)) can be administered to a cell and/or a cardiomyocyte which is present in a subject, for example a human subject. In alternative embodiments, an agent which increase the level of the nuclear form of Creb312 can contact a cell and/or a cardiomyocyte, or a cardiomyocyte progenitor prior to transplanting such a cell and/or cardiomyocyte (or population of those cells) into a subject. In some embodiments, cells are modified to express a Creb312(N) polypeptide or a biologically active fragment thereof, and transplanted into a subject. In some embodiments, a cell can be transduced ex vivo with a nucleic acid encoding a Creb312(N) polypeptide or a biologically active fragment thereof, which is operatively linked to a promoter, and then subsequently transplanted into a subject. In some embodiments, the cells are cardiac progenitor cells, as disclosed in U.S. In some embodiments, the cells are originated (i.e. came from) the same subject from which the cells are transplanted into. Any means to transducer a cell which is known by a person of ordinary skill in the art can be used, for example using vectors, such as plasmids, viral vectors, bacteriophage vectors and the like. In some embodiments of this aspect and all other embodiments described herein, a subject is a mammalian subject, preferably, but not limited to, a human subject.

Another aspect of the present invention relates to methods to identify agents and/or pathways which promote the proteolytic cleavage of a full length Creb312 polypeptide to a nuclear form of Creb312 (Creb312(N)) polypeptide. Accordingly, one aspect of the present invention relates to a method to identify agents which (i) promote the survival of a cell in response to stress, such as mechanical-induced stress, and/or (ii) promote the proliferation of a differentiated cardiomyocyte. Such identified agents are useful as potential therapy to protect a decrease in cardiac function in response to mechanical stress and/or for the treatment to restore cardiac function.

In another aspect of the invention, an agent which increases the activity of at least one signal transduction cascade which results in an increase in the level of a Creb312(N) polypeptide, or a ILK polypeptide, or a PI3-kinase/Akt polypeptide is useful in the methods and compositions as disclosed herein. In some embodiments, agents useful herein include a Creb312(N) polypeptide, a truncated biologically active fragments of a Creb312(N) polypeptide, functional analogs of a Creb312(N) polypeptide and pharmaceutically acceptable derivatives, salts and analogues of a Creb312(N) polypeptide, and combinations thereof.

In another aspect of the invention relates to a method of increasing the proliferation of a post-mitotic cell by contacting such a post-mitotic cell with an agent which activates the cleavage of full length Creb312 polypeptide to a Creb312(N) polypeptide. In some embodiments of this aspect and all aspects described herein, a method of increasing the proliferation of a post-mitotic cell comprises contacting a post-mitotic cell with a nucleic acid encoding a Creb312(N) polypeptide, which is operatively linked to a promoter. In some embodiments of this aspect and all aspects described herein, a method of increasing the proliferation of a post-mitotic cell comprises contacting a post-mitotic cell a Creb312(N) polypeptide, or biologically functional fragment or variants thereof, or pharmaceutically acceptable derivatives or salt thereof. In one embodiment of this aspect, and all other aspects described herein, a post-mitotic cell can be a cardiomyocyte, and preferably a mammalian cardiomyocyte, such as a human cardiomyocyte. In some embodiments, proliferation of a cell comprises at least one of; cell cycle reentry, increased cardiomyocyte DNA synthesis and cytokinesis. In some embodiments, a Creb312(N) polypeptide, fragments or variants thereof, is delivered locally.

In another aspect, the invention discloses a method of inducing division of post mitotic cells, comprising administering an ILK activator or a pharmaceutically acceptable derivative thereof to a subject in an amount effective to stimulate de-differentiation of post-mitotic cells. An ILK activator can be a ILK polypeptide or a biologically functional fragment thereof. In some embodiments, an ILK activator or a pharmaceutically acceptable derivative thereof can be delivered locally to the target cells and/or target area. In some embodiments, local and/or targeted delivery can be administered using a slow controlled release delivery system, such as, for example, a biodegradable matrix. In some embodiments of this aspect and all other aspects described herein, an agent can be used with a long-term, short-term and/or controlled release delivery system.

In another aspect, the invention provides a method of repairing heart tissue, comprising identifying a subject in need of heart tissue repair, and administering to the subject an effective amount of an agent which increases the Creb312(N) polypeptide in a cardiomyocyte, such that proliferation of cardiomyocytes increases. In some embodiments of this aspect and all other aspects described herein, the subject in need of heart tissue repair has undergone myocardial ischemia, hypoxia, stroke, or myocardial infarction.

In some embodiments, the methods and compositions as disclosed herein can be used to treat a subject having, or having an increased risk of developing a cardiovascular condition or disease or injury, for example but not limited to congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, autoimmune endocarditis, and congenital heart disease.

One aspect of the invention relates to a method for increasing the survival of a cell in response to stress, comprising contacting the cell with at least one agent which increases the level of the nuclear form of Creb312 polypeptide in the cell, wherein the nuclear form of Creb312 polypeptide comprises the amino acid residues 1 to 376 of SEQ ID NO: 2 or a biologically active fragment or variant thereof, and wherein the increase in nuclear form of Creb312 polypeptide in the cell increases the survival of the cell in response to stress.

Another aspect of the invention relates to a method for inducing the proliferation of a cardiomyocyte, comprising contacting the cardiomyocyte with at least one agent which increases the level of the nuclear form of Creb312 polypeptide in the cell, wherein the nuclear form of Creb312 polypeptide comprises the amino acid residues 1 to 376 of SEQ ID NO: 2 or a biologically active fragment or variant thereof, and wherein the increase in nuclear form of Creb312 polypeptide induces the proliferation of the cardiomyocyte.

In some embodiments of all aspects described herein, an agent is an activator of the PI3K/Akt pathway, such as for example but not limited to; an activator of the PI3K/Akt pathway is selected from a group consisting of; a ILK polypeptide or an activator of the ILK polypeptide. In one embodiment, an agent comprises a polypeptide of amino acid sequence SEQ ID NO: 3 or a biologically active variant or biologically active fragment thereof.

In some embodiments of all aspects described herein, an agent is a nucleic acid or nucleic acid analogue which encodes a polypeptide comprising amino acid sequence SEQ ID NO: 3 or a functional variant or functional fragment thereof, and in some embodiments, a nucleic acid or nucleic acid analogue can be selected from DNA, RNA, messenger RNA (mRNA), genomic RNA, PNA, pcPNA and Locked nucleic acid (LNA).

In some embodiments of all aspects described herein, an agent is a protease which cleaves the full length Creb312 polypeptide of SEQ ID NO: 2 at SP1 and/or SP2 cleavage sites.

In some embodiments of all aspects described herein, a stress is selected from the group consisting of; hormonal stress, mechanical stress, stretch stress, hypoxic stress.

In some embodiments of all aspects described herein, a cell is a cardiac cell, or a cardiac progenitor cell, or a cardiomyocytes or cardiomyocyte precursor, such as, for example but not limited to a postnatal cardiomyocyte. In some embodiments of all aspects described herein, a cardiomyocyte is a human cardiomyocyte.

In some embodiments of all aspects described herein, a cell, such as a cardiomyocyte can be present in a subject, such as for example but not limited to mammalian subject, preferably a human subject. In some embodiments, a cells is from (i.e. originates) from a subject such as a human subject, for example, a cell can originate from a human subject, be contacted with the agents as disclosed herein, and then subsequently transplanted back into the subject from which the cells originated from.

In some embodiments of all aspects described herein, a subject can have, or have an increased risk of a cardiovascular condition or disease or injury, such as for example, but not limited to a congestive heart failure, a coronary artery disease, a myocardial infarction, a myocardial ischemia, an atherosclerosis, a cardiomyopathy, an idiopathic cardiomyopathy, a cardiac arrhythmia, muscular dystrophy, a muscle mass abnormality, a muscle degeneration, an infective myocarditis, a drug- or toxin-induced muscle abnormality, a hypersensitivity myocarditis, an autoimmune endocarditis, and congenital heart disease.

In some embodiments of all aspects described herein, a cell and/or cardiomyocyte can be transplanted into the subject.

Another aspect of the present invention relates to a composition comprising a Creb312(N) polypeptide or a biologically active variant or fragment thereof, or a pharmaceutically acceptable derivative thereof for use in the manufacturer of a medicament to increase the survival of a cell to mechanical stress.

Another aspect of the present invention relates to a composition comprising a Creb312(N) polypeptide or a biologically active variant or fragment thereof, or a pharmaceutically acceptable derivative thereof for use in the manufacturer of a medicament for treatment of a cardiovascular condition, disease or injury to induce the proliferation of cardiomyocytes.

In some embodiments of this aspect and all aspects described herein, a Creb312(N) polypeptide comprises amino acid residues 1 to 376 of SEQ ID NO: 2 or a biologically active fragment or variant thereof.

Another aspect of the present invention relates to a composition comprising at least one agent which results in the cleavage of full length Creb312 polypeptide to a Creb312(N) polypeptide or a biologically active variant or fragment thereof, in the manufacturer of a medicament to increase the survival of a cell to mechanical stress.

Another aspect of the present invention relates to a composition comprising at least one agent which results in the cleavage of full length Creb312 polypeptide to a Creb312(N) polypeptide or a biologically active variant or fragment thereof, in the manufacturer of a medicament for treatment of a cardiovascular condition, disease or injury to induce the proliferation of cardiomyocytes.

In some embodiments of this aspect and all aspects described herein, an agent useful in the compositions disclosed herein can activate or increase the signalling the PI3K/Akt pathway.

In some embodiments of this aspect and all aspects described herein, the compositions described herein are useful in the treatment of a disease such as, but not limited to In some embodiments of this aspect and all aspects described herein, a disease is selected from the group consisting of congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, autoimmune endocarditis, and congenital heart disease.

In some embodiments of this aspect and all aspects described herein, the compositions disclosed herein are useful to increase the proliferation of a cardiomyocyte cell and/or for the expansion of a population of cardiomyocyte cells. In some embodiments of this aspect and all aspects described herein, a cardiomyocyte is a postnatal cardiomyocyte, such as a mammalian cardiomyocyte, including but not limited to a human cardiomyocyte.

Another aspect of the present invention relates to a method to promote the survival of a transplanted cell in a subject, comprising; (a) contracting the transplanted cell with an agent with at least one agent which increases the level of the nuclear form of Creb312 polypeptide in the transplanted cell, wherein the nuclear form of Creb312 polypeptide comprises the amino acid residues 1 to 376 of SEQ ID NO: 2 or a biologically active fragment or variant thereof, (b) transplanting the cell into a subject; wherein the increase in nuclear form of Creb312 polypeptide in the cell increases the survival of the cell in response to stress.

Another aspect of the present invention relates to a method to promote the survival of a transplanted cell to mechanical stress, comprising; introducing into a transplanted cell a nucleic acid encoding a nuclear form of Creb312 polypeptide which is operatively linked to a promoter, wherein the nuclear form of Creb312 polypeptide comprises the amino acid residues 1 to 376 of SEQ ID NO: 2 or a biologically active fragment or variant thereof; wherein expression of a nuclear form of Creb312 polypeptide increases the survival of the transplanted cell to mechanical stress as compared to a cell not expressing Creb312(N) protein. In some embodiments of this aspect of the invention further comprises a transplanting the cell into a subject.

In some embodiments of this and all other aspects described herein, a cell is contacted with an agent before or after transplantation into a subject. In some embodiments of this and all other aspects described herein, a cell is a cardiac progenitor cell. In some embodiments of this and all other aspects described herein, a promoter is a cardiac promoter or a mechanical stress-induced promoter.

### BRIEF DESCRIPTION OF FIGURES

FIG. 1A-1D shows group of transcription factors that are activated proteolytically by “regulated intramembrane proteolysis” (RIP). FIG. 1A shows the phylogenetic tree of the Creb family of transcription factors, showing the closest family member to Creb312 is OASIS(Creb3L1). FIG. 1B is a schematic representation of members of RIP regulated transcription factor family to demonstrate their protein domains and S1T and S2P cleavage sites, as well as their nuclear localization sites (nls), transmembrane (tm) spanning regions. FIG. 1C shows sequence comparison of a region of OASIS/Creb3L1, Creb3L2, ATF6 and SREBP2 transcription factors, demonstrating the transmembrane spanning region (boxed) and the S2P cleavage site (left hand arrow) and SIT recognition sequence (RxxL, right hand arrow) (where x is any amino acid) showing high homology of the amino acid sequences in the transmembrane domains between Creb3L1 and Creb312. FIG. 1D shows a schematic of construction of variants of Creb312, with the top panel showing the plasmid constructs that express the HA-tagged full-length Creb312 (HA-Creb312FL) and a deletion construct HA-Creb312(ΔTM) that lacks the transmembrane domain and the C-terminal portion. The bottom panel of 1D shows anti-HA immunostaining of expression Cos7 fibroblasts cells transfected with either the HA-Creb312FL and Creb312(ATM) construct. Cells transfected with HA-Creb312FL demonstrates full-length Creb312 is localized in the ER membrane, whereas cells transfected with HA-Creb312(ATM) which lacks the C-terminal and transmembrane show the nuclear form of Creb312 (i.e. amino acids 1-376 of Creb312) is localized exclusively in the nucleus.

FIG. 2A-2E show developmental expression of full length Creb312 and homodimerization of the nuclear form of Creb312. FIG. 2A shows RT-PCR analysis of full length Creb312 in whole embryos is detected by E6.5. FIG. 2B shows the onset of creb312 expression as compared to known secondary heart field cardiac marker genes nkx2.5, isl1, gata4, ggf10, hand1 and tbx5, and indicates that expression of Creb312 transcript is markedly increased in CJ7 ES cells at EB day 6, parallel to the expression of nkx2.5 and tbx5, and increases until EB day 10. FIG. 2C shows immunoprecipitation of FLAG-Creb312 by anti-Flag and Immunoblot with ant-Flag or anti-HA of COS7 cells transfected with either HA-Creb312(ΔTM) or FLAG-Creb312(ΔTM). A positive Immunoblot analysis for anti-HA demonstrates Creb312(ΔTM) or the nuclear form of Creb312 homodimerizes in vitro. FIG. 2D shows immunoprecipitation of FLAG-Creb312(ΔTM) (referred to as Creb312(N) in the figure) and endogenous nuclear form of Creb312 from nuclear extracts in E7.5 whole embryos or E8.5-E12.5 mouse embryonic hearts. Flag-tagged Creb312(N) overexpressed in COS7 cells was used to co-immunoprecipitate endogenous Creb312 from nuclear extracts of E7.5 total embryos or E8.5-E12.5 mouse embryonic hearts. As shown in FIG. 2E, both overexpressed and endogenous Creb312 were then detected with a Creb312-antibody targeted against the N-terminal region. FIG. 2E shows immunoprecipitation of the nuclear form of Creb312 in the mouse embryonic hearts at E8.5 to E12.5, but not at E7.5.

FIG. 3A-3G shows effect of knockout of Creb312 in the mouse. FIG. 3A shows a schematic of the targeting vector for the generation Creb312 knockout mice through targeted homologous recombination. The DNA-binding basic region, the nuclear localization signal (nls) and the leucine zipper domain required for the dimerization were removed in the targeting vector and replaced with an IRES-EGFP reporter as well as a floxed neomycin resistance gene cassette. Four ES cell clones out of 203 screened G418/gancyclovir resistant clones showed the correct targeting and removal of the Creb312 basic region/leucine zipper domain as well as the nuclear localization signal (nls). FIG. 3B shows Southern Blot analysis of homozygous knockout (−/−), heterozygous knockout (+/−) or wild type (+/+) mice. FIG. 3C shows RT-PCR results from E11.5 wildtype (+/+) and knockout (−/−) embryonic hearts demonstrating the absence of functionally active Creb312 mRNA in the knockout hearts. FIG. 3d shows frequency of genotype of progeny from intercrossing of heterozygotes Creb312 mice (+/−), and show that offsprings genotyped at three weeks of age homozygous Creb312 null mutant (−/−) have a lower frequency than expected according to the Mendelian ratio. FIG. 3E shows H&E histological staining of transverse sections of E11.5 and E13.5 embryonic hearts of homozygous Creb312 null mutant (−/−) or wild type (+/+) littermates, showing no gross phenotypic abnormalities in Creb312−/− embryos. FIG. 3E (b,d) shows no gross structural abnormalities such as ventricular septal defects (VSD), or other valve or outflow tract abnormalities in homozygous Creb312 null mutant (−/−) at E11.5. FIG. 3E(d) shows the right ventricular wall of Creb312 null mutant embryos is thinner and hypocellular as compared to wild type right ventricular wall at both E11.5 (compare black lines in FIG. 3E(c) with 3E(d)) and E13.5 (compare the black lines in FIG. 3E(g) with 3E(h)). FIG. 3F shows approximately a 50% increase in TUNEL positive, apoptotic cells in homozygous Creb312 null mutant (−/−) as compared to wild type (+/+) littermate mice at E11.5, and increased (approximately 100% increase) TUNEL staining in the ventricles of Creb312 null mutant (−/−) as compared to wild type (+/+) littermate mice at E13.5, but not a significant increase in TUNEL staining in AV valves of homozygous Creb312 null mutant (−/−) as compared to wild type (+/+) littermate mice at E13.5. FIG. 3G shows no significant change in the number of phospho-H3-positive cells (as detected by anti-phospho H3 immunohistochemistry staining) in ventricular cardiomyocytes or endocardial cushions of wildtype (+/+) and Creb312 null mutant (−/−) hearts at either E11.5 or E13.5.

FIGS. 4A-4G shows proteolytic cleavage of Creb312 by biomechanical stretch stress, hormonal agonists, ER stressors and hypoxia. FIG. 4A shows a schematic to assess which stresses (chemical, hormonal, mechanical and hypoxic stress) lead to the proteolytic cleavage of full length Creb312 to the nuclear (active) form of Creb312. Primary rat neonatal ventricular cardiomyocytes (NRVM) were transduced with recombinant adenovirus expressing the HA-tagged, full-length Creb312 (HA-Creb312(FL)) and subjected them to chemical, hormonal, mechanical and hypoxic stress. FIG. 4B shows Western Blot analysis with an anti-HA antibody demonstrating the formation of the proteolytically cleaved, nuclear form of Creb312 (identified by —N) within 1 hour when brefeldinA (BFA), thapsigargin (TG), DTT and tunicamycin (TG) (known ER stress-inducing chemicals) are applied to NRVM cells expressing HA-Creb312(FL). FIG. 4C shows Western Blot analysis with an anti-HA antibody demonstrating the relatively late formation of the proteolytically cleaved, nuclear form of Creb312 when 1% O₂ (a known hypoxic stress agent) is applied to NRVM cells expressing HA-Creb312(FL) for 24 hrs. The lower panel of FIG. 4C shows Immunoblot analysis using anti-HIFα to confirm the induction of the cellular response to hypoxic stress in the NRVM. FIG. 4D shows Western Blot analysis with an anti-HA antibody demonstrating the formation of the proteolytically cleaved, nuclear form of Creb312 (identified by —N) when NRVM cells expressing HA-Creb312(FL) after 24 hr exposure to hypertrophic agonists such as isoproterenol (ISO) (β1/2 adrenergic agonist), phenylephrine (PE) (α1 adrenergic agonist), angiotensin-2 (AT2) or endothelin-1 (ET1) (GCRP receptor agonist). FIG. 4E shows a schematic of method of mechanical stretch stress of ventricular cardiomyocytes. FIG. 4F shows a Western Blot analysis with an anti-HA antibody demonstrating the formation of the proteolytically cleaved, nuclear form of Creb312 (identified by —N) when NRVM cells expressing HA-Creb312(FL) are subject to 10% pulsary (cyclic) biaxial mechanical stretch for various time periods. FIG. 4G shows Western Blot analysis with an anti-HA antibody demonstrating the formation of the proteolytically cleaved, nuclear form of Creb312 (identified by —N) when NRVM cells expressing HA-Creb312(FL) are subject to either 20% pulsary (cyclic) or 10% static biaxial mechanical stretch for 30 mins or 1 hr.

FIG. 5 shows Creb312 activation by mechanical stretch stress is mediated by the ILK pathway and modulated by PI3K/Akt. FIG. 5 shows Western Blot analysis with an anti-HA antibody demonstrating the inhibitors LY294002 (PI3K inhibitor), SH6, Akt1/2 Inh (Akt inhibitors completely inhibited the formation of the proteolytically cleaved, nuclear form of Creb312 (identified by —N) in response to 10% pulsary (cyclic) biaxial mechanical stretch in NRVM expressing HA-Creb312(FL) cells. Inhibitors PD98059, U0126 (Erk1/2 inhibitor), SP600125 (JNK inhibitor), PP2 (Src inhibitor) and SB203580 (p38/MAPK inhibitor) did not inhibit the formation of the proteolytically cleaved, nuclear form of Creb312 in response to 10% pulsary (cyclic) biaxial mechanical stretch in NRVM expressing HA-Creb312(FL) cells.

FIG. 6 shows a schematic illustration of the stresses which induce the proteolytic cleavage of full length Creb312 to the nuclear(active) form Creb312.

FIG. 7 shows that Creb312 activates BNP gene transcription. FIG. 7 shows expression of luciferase protein which is regulated by the BNP promoter region in response to different kinase stress proteins such as c-fos, c-jun, MAKF, ATF1 and Creb312, tested at 10, 50, 20 or 100 ng plasmid DNA encoding the transcription factor. Creb212 induces BNP regulated luciferase gene expression at 20 and 100 ng Creb312 DNA.

FIGS. 8A-8D show the nuclear form of Creb312 activates BNP regulated luciferase gene expression. FIG. 8A shows a schematic of the transcription binding sites for ANF and BNP, as well as the BNP fragment (BNP(−135)) that is operatively linked to the luciferase gene for a luciferase gene expression assay. FIG. 8B shows a 700 bp promoter region of ANF regulated luciferase gene expression induced by the nuclear form of Creb312 (Creb312™) at 50 ng DNA, but not full length Creb312 (Creb312FL). FIG. 8C shows a 2 kb promoter region of BNP regulated luciferase gene expression induced by the nuclear form of Creb312 (Creb312™) at 10 ng DNA, but not full length Creb312 (Creb312FL). FIG. 8D shows a 135 bp promoter region of BNP regulated luciferase gene expression induced by the nuclear form of Creb312 (Creb312™) at 10 ng DNA, but not full length Creb312 (Creb312FL).

FIG. 9 shows fold activation of luciferase by Creb312(TM) when luciferase is operatively linked to different or mutated BNP promoter regions. FIG. 9 shows a schematic of the different transcription binding sites in the BNP promoter regions, and their presence, absence or mutated variants mediate the ability of nuclear form of Creb312 (Creb312(TM)) to induce luciferase expression. Deletion analysis demonstrates the nuclear form of Creb312 binding site is −135 residues upstream of the BNP initiation site (ATG site), and a −135 BNP promoter fragment with a mutated 3′-end to include TTA after the TCCTGAGCTCAGC (SEQ ID NO: 8) is highly effective at binding Creb312 and inducing BNP-promoter driven transcription of luciferase.

FIG. 10A-10C shows deletion analysis to determine the region of the BNP promoter which comprises the nuclear form of Creb312 (Creb312(TM)) binding site. FIG. 10A shows a schematic of BNP promoter regions; (−135 to −115) or (−129 to −110) operatively linked to the luciferase gene. FIG. 10B shows fold activation of luciferase gene expression in response to increasing concentrations of the nuclear form of Creb312 (Creb312(TM)) when the −135 BNP promoter region is operatively linked to the luciferase gene expression. FIG. 10C shows fold activation of luciferase gene expression in response to increasing concentrations of the nuclear form of Creb312 (Creb312(TM)) when either the (−135 to −115) or (−129 to −110) BNP promoter regions are operatively linked to the luciferase gene expression. A three fold increase in activation is demonstrated in the presence of 200 ng DNA of nuclear form of Creb312 (Creb312(TM)) when the (−135 to −115) BNP promoter region is operatively linked to the luciferase gene.

### DETAILED DESCRIPTION

The present invention relates to the inventors discovery that the nuclear form of the transcription factor Creb312 functions to promote survival of cells in response to cellular stress, such as cardiac cells from mechanical-induced stretch stress. As disclosed herein, the inventors have demonstrated that Creb312, a novel sarcoplasmic reticulum (SR) embedded transcription factor of the basic region/leucine zipper (Creb312) family, is enriched in heart progenitors in vivo and in vitro, and later in diverse differentiated progeny, including cardiomyocytes. The inventors demonstrate that concurrent with the onset of the heart beat in vivo and in response to unibiaxial mechanical stretch stress in vitro, the SR membrane-bound Creb312 protein is activated through proteolytic cleavage and the N-terminal portion of Creb312 (herein referred to as “Creb312(N)” or the “nuclear form of Creb312”) translocates to the nucleus, where it can trans-activate the expression of cell survival genes, such as stretch-induced target genes.

Creb312 is initially expressed as a ER-localized transmembrane protein and is proteolytically processed into a transcriptionally active nuclear form, herein referred to “nuclear form of Creb312” after mechanical stress stimuli. The inventors have demonstrated using BrdU labelling and immunocytochemistry experiments with proliferative markers, the inventors demonstrate that Creb312 activates re-entry into cell cycle and proliferation of primary neonatal rat cardiomyocytes by five fold (discussed in more detail below). The inventors also demonstrate that this is important in mammalian hearts, as cardiomyocytes exit the cell cycle and lose their proliferative capacity shortly after birth. As a consequence, any injuries to the heart or heart injures associated with a decrease in cardiomyocyte numbers and myocardial infarct and ischemia-reperfusion is invariably linked to the irreversible decrease in cardiomyocyte numbers and reduced physiological heart function.

Accordingly, the inventors have discovered that the nuclear form of Creb312 functions (i.e. amino acids 1-376 of full length Creb312 corresponding to SEQ ID NO: 2) as a mechanical stress response survival factor. The inventors have discovered a method to prevent mechanical stretch stress induced cell death of cardiac cells, in particular heart progenitors and their differentiated progeny, such as cardiomyocytes. Thus, one aspect of the present invention relates to a method to activate full length Creb312 (corresponding to SEQ ID NO: 2) into the nuclear Creb312 functional form (corresponding to SEQ ID NO: 3), for example, a method to promote the cleavage of full length Creb312 to the nuclear Creb312 to promote the survival of heart progenitors and cardiomyocytes from mechanical stretch activated stress.

The inventors have also demonstrated that the proteolytic activation of full length Creb312 (SEQ ID NO: 2) into the nuclear form of Creb312 (SEQ ID NO: 3) is dependent on known PI3 kinase dependent signals that mediate mechanical stretch induced signals in cardiomyocytes. In particular, the inventors have demonstrated that cleavage of full length Creb312 into nuclear Creb312 is dependent on the PI3K/Akt pathway and formation of nuclear form of Creb312 is prevented in the presence of inhibitors of PI3K and/or Akt.

The inventors further demonstrate that the loss of full length Creb312 in murine embryos leads to partial embryonic lethality due to an increased apoptosis of right ventricular cardiomyocytes and atrioventricular valves, and thinning of the right ventricular myocardial wall, consistent with the loss of myocyte survival. The small number of Creb312 deficient neonates which survive ultimately display cardiomyopathy as a result of the loss of cardiomyocytes during fetal cardiogenesis. While not wishing to be bound by theory, it is believed that this is due to the fact that during the conversion of the early cardiovascular progenitors to their differentiated progeny, the fetal heart is exposed to a variety of hormonal, physiological, and mechanical stress signals during the initial formation of the heartbeat, and the absence of the nuclear form of Creb312 in Creb312 deficient neonates results in loss of cardiomyocytes during fetal cardiogenesis and consequently cardiomyopathy. Accordingly, the inventors herein have demonstrated that the nuclear form of Creb312 functions as a pivotal survival protein for heart progenitors and their differentiated progeny during development.

Stated in another way, the inventors have discovered a novel protective mechanism against cellular stress, such as mechanical stress during embryonic heart development, the loss of which leads to a form of cardiomyopathy in the fetal heart. The inventors have discovered a novel pathway to preserve cardiomyocyte progenitor survival in vivo (i.e. in a living cell) to enhance the regenerative capacity of progenitors, such as cardiomyocyte progenitors in cell-based therapy paradigms.

Accordingly, the inventors provide a method to activate full length Creb312 (Creb312(FL)) to produce a nuclear form of Creb312 (Creb312(N)) which can be used to (i) protect developing cardiomyocytes and/or cardiomyocyte precursors from mechanical stretch-induced stress, as well as (ii) induce and stimulate the proliferation of otherwise quiescent cardiomyocytes for regeneration of postnatal cardiomyocytes for various uses, including but not limited to therapeutic use where an increase of cardiomyocytes a desired outcome, such as in cases where degeneration of cardiomyocytes has occurred or due to injury or disease associated with decrease in cardiomyocyte numbers.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “Creb312” is also referred to in the art as “Creb3L2” and the human Creb312 protein is encoded by the nucleic acid sequence corresponding to SEQ ID NO: 1 (i.e. RefSeqID No: NM_{—}194071, GeneID No: 64764, Accession No: AJ549092). Creb312 is also known as aliases; Creb3L2, cAMP responsive element binding protein 3-like 2, BBF2H7 and TCAG_{—}1951439. The human full length Creb312 polypeptide corresponds to RefSeq ID No: NP_{—}919047, GeneID No: 64764, herein referred to as SEQ ID NO: 2. The nuclear form of human Creb312 is residues 1-376 of the full length Creb312 and corresponds to SEQ ID NO: 3, and is also referred to herein as “Creb312(N)” or “Creb312(N)” or “Creb312(ΔTM)” or the N-terminal Creb312 polypeptide.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “protease” is a polypeptide that cleaves another polypeptide at a particular site (amino acid sequence). The protease can also be self-cleaving. A protease is said to be “specific” for another polypeptide when it characteristically cleaves the other “substrate” polypeptide at a particular amino acid sequence. The specificity can be absolute or partial (i.e., a preference for a particular amino acid or amino acid sequence).

The term “specifically binds” when used to refer to binding proteins herein indicates that the binding preference (e.g., affinity for the target molecule/sequence is at least 2 fold, more preferably at least 5 fold, and most preferably at least 10 or 20 fold over a non-specific (e.g. randomly generated molecule lacking the specifically recognized amino acid or amino acid sequence) target molecule.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The term “wild type” refers to the naturally-occurring polynucleotide sequence encoding a protein, or a portion thereof, or a naturally-occurring protein sequence, or portion thereof, respectively, as it normally exists in vivo.

The term “mutation”, when used in reference to a polypeptide refers to the change of one or more amino acid residues in a polypeptide to residues other than those found in the “native” or “reference (pre-mutation) form of that polypeptide. Mutations include amino acid substitutions as well as insertions and/or deletions. A mutation does not require that the particular amino acid substitution or deletion be made to an already formed polypeptide, but contemplates that the “mutated” polypeptide can be synthesized de novo, e.g. through chemical synthesis or recombinant means. It will be appreciated that the mutation can include replacement of a natural amino acid with an “unnatural” amino acid.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

The term “variant” as used herein refers to a peptide or nucleic acid that differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide). A “variant” of a Creb312(N) polypeptide, for example SEQ ID NO: 3 is meant to refer to a molecule substantially similar in structure and function, i.e. where the function is the ability to protect cells against mechanical stretch-induced stress, and/or induce the expression of genes regulated by the BNP response element, or increase the proliferation of cardiomyocytes.

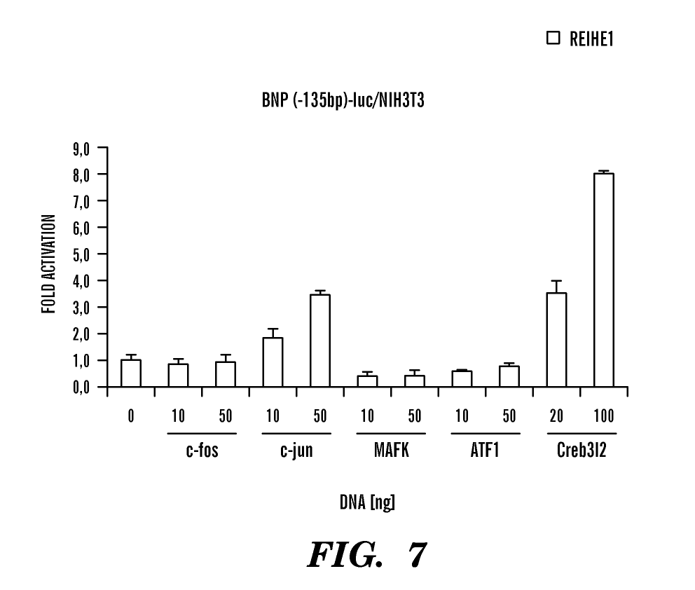

Variants can be naturally-occurring, synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. ability of the Creb312(N) variant protein to increase cell survival in response to mechanical stretch-induced stress, or to increase BNP-mediated induction of luciferase expression using the BNP-luciferase in vitro assay as disclosed herein in the Examples and in FIG. 7 as compared to a non variarant Creb312(N) polypeptide of SEQ ID NO:3). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In some embodiments, individual substitutions, deletions or additions that alter, add or delete a single amino acid or a small percentage of amino acids can also be considered “conservative substitutions” is the change does not reduce the activity of the peptide (i.e. the ability of an Creb312(N) polypeptide variant to to promote survival of cells to mechanical stretch-induced stress or promote the proliferation of cardiomyocytes). Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

The term “conservative substitution” is used in reference to proteins or peptides to reflect amino acid substitutions that do not substantially alter the activity (specificity or binding affinity) of the molecule. Typically conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The term “functional” when used in conjunction with “derivative” or “variant” refers to a molecule such as a protein which possess a biological activity (either functional or structural) that is substantially similar to a biological activity of the entity or molecule its is a functional derivative or functional variant thereof. The term functional derivative is intended to include the fragments, analogues or chemical derivatives of a molecule.

A molecule is said to be “substantially similar” to another molecule if both molecules have substantially similar structures or if both molecules possess a similar biological activity, for example if both molecules are able to protect cells against mechanical stretch-induced stress, and/or induce the expression of genes regulated by the BNP response element, or increase the proliferation of cardiomyocytes. Thus, provided that two molecules possess a similar activity, (i.e. a variant of a Creb312(N) polypeptide which can protect cells against mechanical stretch-induced stress, and/or induce the expression of genes regulated by the BNP response element, or increase the proliferation of cardiomyocytes similar to that of the Creb312(N) polypeptide which corresponds to SEQ ID NO: 3) are considered variants and are encompassed for use as disclosed herein, even if the structure of one of the molecules not found in the other, or if the sequence of amino acid residues is not identical. Thus, provided that two molecules possess a similar biological activity, they are considered variants as that term is used herein even if the structure of one of the molecules not found in the other, or if the sequence of amino acid residues is not identical.

As used herein, the term “nonconservative” refers to substituting an amino acid residue for a different amino acid residue that has different chemical properties. The nonconservative substitutions include, but are not limited to aspartic acid (D) being replaced with glycine (G); asparagine (N) being replaced with lysine (K); or alanine (A) being replaced with arginine (R).

The term “insertions” or “deletions” are typically in the range of about 1 to 5 amino acids. The variation allowed can be experimentally determined by producing the peptide synthetically while systematically making insertions, deletions, or substitutions of nucleotides in the sequence using recombinant DNA techniques.

The term “substitution” when referring to a peptide, refers to a change in an amino acid for a different entity, for example another amino acid or amino-acid moiety. Substitutions can be conservative or non-conservative substitutions.

The term “fragment” of a polypeptide or molecule as used herein refers to any contiguous polypeptide subset of the molecule. Fragments of an Creb312(N) polypeptide, for example functional fragments of SEQ ID NO: 3 useful in the methods as disclosed herein have at least 30% of agonist or antagonist activity as that of SEQ ID NO: 3. Stated another way, a fragment of a Creb312(N) polypeptide is a fragment of SEQ ID NO: 3 which can result in at least 30% of the same activity as compared to the polypeptide of SEQ ID NO: 3 to are able to protect cells against mechanical stretch-induced stress, and/or induce the expression of genes regulated by the BNP response element, or increase the proliferation of cardiomyocytes (as disclosed in the Examples herein) and/or results in at least 30% of the activity as compared with SEQ ID NO: 3 to increase BNP-mediated induction of luciferase expression using the BNP-luciferase in vitro assay as disclosed herein in the Examples and in FIG. 7. A fragment of Creb312(N) can also include fragments that decrease the wild type activity of one property by at least 30%. Fragments as used herein are soluble (i.e. not membrane bound), and in some embodiments can be bound to a first fusion partner, however, they do not need to be fused to a fusion protein if the fragment of Creb312(N) is stable. A “fragment” can be at least about 6, at least about 9, at least about 15, at least about 20, at least about 30, least about 40, at least about 50, at least about 100, at least about 250, at least about 500 nucleic or amino acids, and all integers in between. Exemplary fragments include C-terminal truncations, N-terminal truncations, or truncations of both C- and N-terminals (e.g., deletions of, for example, at least 1, at least 2, at least 3, at least 4, at least 5, at least 8, at least 10, at least 15, at least 20, at least 25, at least 40, at least 50, at least 75, at least 100 or more amino acids deleted from the N-termini, the C-termini, or both). One of ordinary skill in the art can create such fragments by simple deletion analysis. Such a fragment of SEQ ID NO: 3 can be, for example, 1, 2, 3, 4, 5, 6, 7, 8, 9 or 10 amino acids or more than 10 amino acids, such as 15, 30, 50, 100 or more than 100 amino acids deleted from the N-terminal and/or C-terminal of SEQ ID NO: 3, respectively. Persons of ordinary skill in the art can easily identify the minimal peptide fragment of SEQ ID NO: 3 useful in the compositions and methods as disclosed herein, as shown by FIG. 7, by sequentially deleting N- and/or C-terminal amino acids from SEQ ID NO: 3 and assessing the function of the resulting Creb312(N) polypeptide fragment to induce luciferase expression in the BNP-assay as disclosed herein, or to protect cells (i.e. promote cell survival) from mechanical stretch-induced stress, or promote the proliferation of cardiomyocytes. One can create functional fragments with multiple smaller fragments. These can be attached by bridging peptide linkers. One can readily select linkers to maintain wild type conformation. One of ordinary skill in the art can easily assess the function of a Creb312(N) polypeptide fragment to protect cells (i.e. promote cell survival) from mechanical stretch-induced stress, or promote the proliferation of cardiomyocytes. as disclosed in the Examples and figures herein) as compared to Creb312(N) corresponding to SEQ ID NO: 3 as disclosed herein. Where one using such an in vivo BNP-luciferase reporter assay, if the Creb312(N) fragment protein has at least 30% of the biological activity of the Creb312(N) corresponding to SEQ ID NO: 2 as disclosed herein, then the Creb312(N) fragment protein is considered a valid Creb312(N)-fragment and can used in the compositions and methods as disclosed herein. Alternatively, one of ordinary skill in the art can easily assess the function of the Creb312(N) fragment protein by assessing its ability to increase the proliferation of cardiomyocytes as compared to Creb312(N) polypeptide corresponding to SEQ ID NO: 3 as disclosed herein, or to determine the ability of the Creb312(N) fragment protein to increase cell survival in response to mechanical stretch-induced stress, or to increase BNP-mediated induction of luciferase expression using the BNP-luciferase in vitro assay as disclosed herein in the Examples and in FIG. 7. Using such an in vitro assay, if the Creb312(N) fragment protein has at least 30% of the biological activity of the Creb312(N) corresponding to SEQ ID NO: 2 as disclosed herein, then the Creb312(N) fragment portion is considered a valid Creb312(N) fragment and can used in the methods and compositions as disclosed herein. In some embodiments, a fragment of Creb312(N) can be fused to heterologous proteins. In some embodiments, a fragment of SEQ ID NO: 3 can be less than 200, or less than 150 or less than 100, or less than 50, or less than 20 amino acids of SEQ ID NOS: 2, 3, 4 or 5. In some embodiments, a fragment of SEQ ID NO: 3 or 5 is less than 100 peptides in length. However, as stated above, the fragment must be at least 6 amino acids, at least about 9, at least about 15, at least about 20, at least about 30, at least about 40, at least about 50, at least about 100, at least about 250, at least about 500 nucleic acids or amino acids, or any integers in between.

As used herein, the term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “nucleic acid” or “oligonucleotide” or “polynucleotide” used herein can mean at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions. The terms “nucleic acid” can also refer to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O- and N-alkylated nucleotides, e.g. N6-methyl adenosine are suitable. The 2′OH— group can be replaced by a group selected from H. OR, R. halo, SH, SR, NH₂, NHR, NR₂ or CN, wherein R is C—C6 alkyl, alkenyl or alkynyl and halo is F, Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

The term “modulate” when used with respect to protease activity refers to an alteration in the rate of reaction (protein hydrolysis) catalyzed by a protease. An increase in protease activity results in an increase in the rate of substrate hydrolysis at a particular protease concentration and a protease modulator that produces such an increase in protease activity is referred to as an “activator” or “protease agonist”. The terms “activator” or “agonist” are thus used synonymously. A decrease in protease activity refers to a decrease in the rate of substrate hydrolysis at a particular protease concentration. Such a decrease may involve total elimination of protease activity. A protease modulator that produces a decrease in protease activity is referred to as a “protease inhibitor”. It will be appreciated that generally the increase or decrease is as compared to the protease absent the protease modulator.

The term “antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50 70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist e.g., as intact immunoglobulins or as a number of well-characterized fragments produced by digestion with various peptidases. Thus, for example, pepsin digests an antibody below the disulfide linkages in the hinge region to produce F(ab)'₂, a dimer of Fab which itself is a light chain joined to V_{H}-C_{H1} by a disulfide bond. The F(ab)'₂ may be reduced under mild conditions to break the disulfide linkage in the hinge region, thereby converting the F(ab)'₂ dimer into an Fab′ monomer. The Fab′ monomer is essentially an Fab with part of the hinge region (see, Fundamental Immunology, Third Edition, W. E. Paul, ed., Raven Press, N.Y. 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies, those synthesized de novo using recombinant DNA methodologies (e.g., single chain Fv), and those found in display libraries (e.g. phage display libraries).

The phrases “hybridizing specifically to” or “specific hybridization” or “selectively hybridize to”, refer to the binding, duplexing, or hybridizing of a nucleic acid molecule preferentially to a particular nucleotide sequence under stringent conditions when that sequence is present in a complex mixture (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes part I chapter 2 Overview of principles of hybridization and the strategy of nucleic acid probe assays, Elsevier, N.Y. Generally, highly stringent hybridization and wash conditions are selected to be about 5 C lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or northern blot is 50% formamide with 1 mg of heparin at 42 C, with the hybridization being carried out overnight. An example of highly stringent wash conditions is 0.15 M NaCl at 72 C for about 15 minutes. An example of stringent wash conditions is a 0.2×SSC wash at 65 C for 15 minutes (see, Sambrook et al. (1989) Molecular Cloning—A Laboratory Manual (2nd ed.) Vol. 13, Cold Spring Harbor Laboratory, Cold Spring Harbor Press, NY, (Sambrook et al.) supra for a description of SSC buffer). Often, a high stringency wash is preceded by a low stringency wash to remove background probe signal. An example medium stringency wash for a duplex of, e.g., more than 100 nucleotides, is 1×SSC at 45 C for 15 minutes. An example low stringency wash for a duplex of, e.g., more than 100 nucleotides, is 4 6 X SSC at 40 C for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

In one particularly preferred embodiment, stringent conditions are characterized by hybridization in 1 M NaCl, 10 mM Tris-HCl, pH 8.0, 0.01% Triton X-100, 0.1 mg/ml fragmented herring sperm DNA with hybridization at 45.degree. C. with rotation at 50 RPM followed by washing first in 0.9 M NaCl, 0.06 M NaH.sub.2PO.sub.4, 0.006 M EDTA, 0.01% Tween-20 at 45° C. for 1 hr, followed by 0.075 M NaCl, 0.005 M NaH₂PO₄, 0.5 mM EDTA at 45° C. for 15 minutes.

The phrase “substantially identical,” in the context of two nucleic acid sequences or polypeptide sequences, refers to two or more sequences or subsequences that have at least 60%, at least 80%, or between 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with the sequences as disclosed herein.

As used herein, the term “sequence identity” means that two polynucleotide sequences or two amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85% sequence identity, preferably at least 90% to 95% sequence identity, more usually at least 99% sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which can include deletions or additions which total 20 percent or less of the reference sequence over the comparison window. The reference sequence can be a subset of a larger sequence. The term “similarity”, when used to describe a polypeptide, is determined by comparing the amino acid sequence and the conserved amino acid substitutes of one polypeptide to the sequence of a second polypeptide.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan.

The term “substantially homologous” refers to sequences that are at least 90%, at least 95% identical, at least 96%, identical at least 97% identical, at least 98% identical or at least 99% identical. Homologous sequences can be the same functional gene in different species. Determination of homologs of the genes or peptides of the present invention can be easily ascertained by the skilled artisan.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351 360. The method used is similar to the method described by Higgins & Sharp (1989) CABIOS 5: 151 153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al (1990) J. Mol. Biol. 215:403 410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et at, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always>0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTIN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff& Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873 5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “biological sample” refers to sample is a sample of biological tissue, cells, or fluid that, in a healthy and/or pathological state, contains a nucleic acid or polypeptide that is to be detected according to the assays described herein. Such samples include, but are not limited to, cultured cells, primary cell preparations, sputum, amniotic fluid, blood, tissue or fine needle biopsy samples, urine, peritoneal fluid, and pleural fluid, or cells therefrom. Biological samples may also include sections of tissues (e.g., frozen sections taken for histological purposes). Although the sample is typically taken from a human patient, the biological sample can be from as any mammal, such as dogs, cats, sheep, cattle, and pigs, etc. The sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

The term “recombinant,” as used herein, means that a protein is expressed by a prokaryotic or eukaryotic expression system.

As used herein, the term “vector” refers to a nucleic acid molecule capable of transporting another nucleic acid to which it has been operatively linked. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors can integrate into the host's genome or replicate autonomously in the particular cell. Other forms of expression vectors known by those skilled in the art which serve the equivalent functions can also be used. Expression vectors comprise expression vectors for stable or transient expression encoding the DNA. A vector can be a plasmid, bacteriophage, bacterial artificial chromosome or yeast artificial chromosome. A vector can be a DNA or RNA vector. A vector can be either a self replicating extrachromosomal vector or a vector which integrate into a host genome.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “regulatory sequence” and “promoter” are used interchangeably herein, refers to a generic term used throughout the specification to refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “reprogramming” as that them is defined herein.

The term “progenitor” or “progenitor cell” is used synonymously with “stem cell.” Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate. It is possible that cells that begin as progenitor cells might proceed toward a differentiated phenotype, but then “reverse” and re-express the progenitor cell phenotype.

The term “reprogramming” as used herein refers to the transition of a differentiated cell to become a progenitor cell. Stated another way, the term reprogramming refers to the transition of a differentiated cell to an earlier developmental phenotype or developmental stage. A “reprogrammed cell” is a cell that has reversed or retraced all, or part of its developmental differentiation pathway to become a progenitor cell. Thus, a differentiated cell (which can only produce daughter cells of a predetermined phenotype or cell linage) or a terminally differentiated cell (which can not divide) can be reprogrammed to an earlier developmental stage and become a progenitor cell, which can both self renew and give rise to differentiated or undifferentiated daughter cells. The daughter cells themselves can be induced to proliferate and produce progeny that subsequently differentiate into one or more mature cell types, while also retaining one or more cells with parental developmental potential. The term reprogramming is also commonly referred to as retrodifferentiation or dedifferentiation in the art. A “reprogrammed cell” is also sometimes referred to in the art as an “induced pluripotent stem” or “iPS” cell.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an atrial precursor), and then to an end-stage differentiated cell, such as atrial cardiomyocytes or smooth muscle cells which plays a characteristic role in a certain tissue type, and may or may not retain the capacity to proliferate further.

As indicated above, there are different levels or classes of cells falling under the general definition of a “stem cell.” These are “totipotent,” “pluripotent” and “multipotent” stem cells. The term “totipotent” refers to a stem cell that can give rise to any tissue or cell type in the body. “Pluripotent” stem cells can give rise to any type of cell in the body except germ line cells. Stem cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood.

The term “differentiation” in the present context means the formation of cells expressing markers known to be associated with cells that are more specialized and closer to becoming terminally differentiated cells incapable of further differentiation. The pathway along which cells progress from a less committed cell, to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a more specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that in the context of this specification, the terms “differentiation” or “differentiated” refer to cells that are more specialized in their fate or function than at one time in their development.

The term “enriching” is used synonymously with “isolating” cells, and means that the yield (fraction) of cells of one type is increased over the fraction of cells of that type as compared to the starting culture or preparation.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are not stem cells or stem cell progeny.

The development of a cell from an uncommitted cell (for example, a stem cell), to a cell with an increasing degree of commitment to a particular differentiated cell type, and finally to a terminally differentiated cell is known as progressive differentiation or progressive commitment. A cell that is “differentiated” relative to a progenitor cell has one or more phenotypic differences relative to that progenitor cell. Phenotypic differences include, but are not limited to morphologic differences and differences in gene expression and biological activity, including not only the presence or absence of an expressed marker, but also differences in the amount of a marker and differences in the co-expression patterns of a set of markers.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

A “marker” as used herein describes the characteristics and/or phenotype of a cell. Markers can be used for selection of cells comprising characteristics of interest. Markers vary with specific cells. Markers are characteristics, whether morphological, functional or biochemical (enzymatic) characteristics particular to a cell type, or molecules expressed by the cell type. Preferably, such markers are proteins, and more preferably, possess an epitope for antibodies or other binding molecules available in the art. A marker may consist of any molecule found in, or on the surface of a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method commonly available to one of skill in the art.

A “reporter gene” as used herein encompasses any gene that is genetically introduced into a cell that adds to the phenotype of the stem cell. Reporter genes as disclosed in this invention are intended to encompass fluorescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny.

The term “lineages” as used herein refers to a term to describe cells with a common ancestry, for example cells that are derived from the same cardiovascular stem cell or other stem cell.

The term “phenotype” refers to one or a number of total biological characteristics that define the cell or organism under a particular set of environmental conditions and factors, regardless of the actual genotype.

The term “tissue” refers to a group or layer of similarly specialized cells which together perform certain special functions. The term “tissue-specific” refers to a source or defining characteristic of cells from a specific tissue.

The terms “subject” and “individual” are used interchangeably herein, and refer to an animal, for example a human, to whom treatment, including prophylactic treatment, with methods and compositions described herein, is or are provided. For treatment of those infections, conditions or disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates.

The term “regeneration” means regrowth of a cell population, organ or tissue after disease or trauma.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “disease” or “disorder” is used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition or affection.

The term “pathology” as used herein, refers to symptoms, for example, structural and functional changes in a cell, tissue or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

With reference to the treatment of a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick J P, Am J Physiol Heart circ Physiol 285; H1797; 2003) and animal models acute myocardial infarction. (Yang Z, Am J Physiol Heart Circ. Physiol 282:H949:2002; Guo Y, J Mol Cell Cardiol 33; 825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals. By “earlier” is meant that a decrease, for example in the size of the tumor occurs at least 5% earlier, but preferably more, e.g., one day earlier, two days earlier, 3 days earlier, or more.

As used herein, the terms “treat” or “treatment” or “treating” refers to both therapeutic treatment and prophylactic or preventative measures, wherein the object is to prevent or slow the development of the disease, such as slow down the development of a cardiac disorder, or reducing at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Accordingly, the term “treating” when used in reference to a cardiovascular disease treatment is used to refer to the reduction of a symptom and/or a biochemical marker of cardiac disease, for example a reduction in at least one biochemical marker of cancer by at least about 10% would be considered an effective treatment. Examples of such biochemical markers of cardiovascular disease include a reduction of, for example, creatine phosphokinase (CPK), aspartate aminotransferase (AST), lactate dehydrogenase (LDH) in the blood, and/or a decrease in a symptom of cardiovascular disease and/or an improvement in blood flow and cardiac function as determined by someone of ordinary skill in the art as measured by electrocardiogram (ECG or EKG), or echocardiogram (heart ultrasound), Doppler ultrasound and nuclear medicine imaging. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

Subjects amenable to treatment by the methods as disclosed herein can be identified by any method to diagnose myocardial infarction (commonly referred to as a heart attack) commonly known by persons of ordinary skill in the art are amenable to treatment using the methods as disclosed herein, and such diagnostic methods include, for example but are not limited to; (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

The term “effective amount” as used herein refers to the amount of therapeutic agent of pharmaceutical composition to reduce at least one or more symptom(s) of the disease or disorder, and relates to a sufficient amount of pharmacological composition to provide the desired effect. The phrase “therapeutically effective amount” as used herein, e.g., of an agent which increases the level of nuclear form of Creb312 in a cell as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylatically significant reduction in a symptom or clinical marker associated with a cardiac dysfunction or disorder when administered to a typical subject who has a cardiovascular condition, disease or disorder.

A therapeutically or prophylatically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

As used herein, the terms “administering,” “introducing” and “transplanting” are used interchangeably and refer to the placement of an agent which increases the level of the nuclear form of Creb312 as described herein into a subject by a method or route which results in at least partial localization of the agent at a desired site. An agent which increases the level of nuclear Creb312 can be administered by any appropriate route which results in effective treatment in the subject, i.e. administration results in delivery to a desired location in the subject where at least a portion of the composition delivered, i.e. at least one agent which increases nuclear Creb312 is active in the desired site for a period of time. The period of time the agent is active depends on the agents half life in vivo after administration to a subject, and can be as short as a few hours, e.g. twenty-four hours, to a few days, to as long as several years.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of atrial progenitors or atrial myocytes and/or their progeny and/or compound and/or other material other than directly into the cardiac tissue, such that it enters the animal's system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The term “drug” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “agent” refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An gent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

Other than in the operating examples, or where otherwise indicated, all numbers expressing quantities of ingredients or reaction conditions used herein should be understood as modified in all instances by the term “about.” The term “about” when used in connection with percentages may mean±1%. The present invention is further explained in detail by the following examples, but the scope of the invention should not limit thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The articles “a” and “an” are used herein to refer to one or to more than one (i.e., to at least one) of the grammatical object of the article. By way of example, “an element” means one element or more than one element.

As used herein, the term “comprising” means that other elements can also be present in addition to the defined elements presented. The use of “comprising” indicates inclusion rather than limitation. Accordingly, the terms “comprising” means “including principally, but not necessary solely”. Furthermore, variation of the word “comprising”, such as “comprise” and “comprises”, have correspondingly the same meanings. The term “consisting essentially of means “including principally, but not necessary solely at least one”, and as such, is intended to mean a “selection of one or more, and in any combination”. Stated another way, the term “consisting essentially of” means that an element can be added, subtracted or substituted without materially affecting the novel characteristics of the invention. This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

Agents that Activate Creb312 to Become the Nuclear Form of Creb312

As discussed above, one aspect of the present invention relates to methods and compositions to promote the survival of cells in response to cellular stress, such as mechanical stretch stress. in particular, methods and compositions to increase the level of the nuclear form of Creb312 polypeptide in a cell where it is desired to increase the survival of the cell in response to mechanical stress. Another aspect of the present invention relates the methods and compositions for inducing the proliferation of cardiomyocytes, such as postnatal cardiomyocytes, adult cardiomyocytes or cardiomyocyte progenitors.

Any means to increase the level of the nuclear form of Creb312 in the cell or cardiomyocyte can be use in the methods of the present invention, including but not limited to, contacting the cell with an agent or entity which increases the level of the nuclear form of Creb312 within the cell. In some embodiments, agents useful in the present invention include, but without limitation, small molecules, nucleic acids, nucleic acid analogues, proteins, protein fragments, aptamers, antibodies etc as discussed herein in more detail.

In some embodiments, the level of nuclear form of Creb312 polypeptide can be increased in a cell or cardiomyocyte by conventional gene therapy methods, as commonly known by persons of ordinary skill in the art, and discussed herein. For example, in some embodiments a nucleic acid encoding the Creb312(N) polypeptide is introduced into the cell, where the nucleic acid is operatively linked to a promoter for expression of the Creb312(N) polypeptide in the cell. Accordingly, the present invention also encompasses gene theapy methods to introduce a nucleic acid encoding Creb312(N) polypeptide into a cell, such as a cardiac progenitor cell, or post mitotic cardiomyocyte for promoting survival of the cells in response to stress and/or promoting proliferation, for example, promoting the proliferation of cardiomyocytes, such as postnatal cardiomyocytes, adult cardiomyocytes or cardiomyocyte progenitors. In some embodiments, a nucleic acid encoding a Creb312(N) polypeptide is introduced into a cardiac progenitor, such as a human Isl1+ primordial cardiac progenitor or their progeny, as disclosed in U.S. Provisional applications 61/185,752 or 61/256,960 which are incorporated herein in their entirety by reference. In some embodiments, a nucleic acid encoding a Creb312(N) polypeptide is introduced into a cardiac progenitor, such as a Isl1+ cardiac progenitor or their progeny as disclosed in International Applications WO/2008/054819 and WO/2009/114673, which are incorporated herein in their entirety by reference.

In some embodiments, the agent comprises a nucleic acid which encodes the polypeptide at least 80% sequence identity to SEQ ID NO: 3, or alternatively at least 85%, or at least 90%, or at least 95%, or at least 98%, or at least 99%, or at least 100% sequence identity to SEQ ID NO: 3. In some embodiments, the agent is a protease, such as a site-1 protease (SIP) and/or a site-2 protease (S2P), which can cleave full length Creb312 (SEQ ID NO: 2) into the nuclear (active) form of Creb312 (i.e. residues 1-376 of SEQ ID NO: 2). In alternative embodiments, the agent is any agent which activates the PI3K/Akt(PKB) pathway.

Protein or Polypeptide Based Agents which Increase Levels of Nuclear Form of Creb312
1) Nuclear form of Creb312

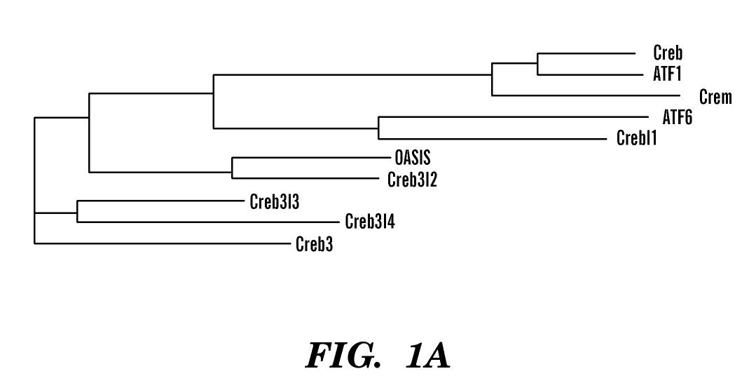

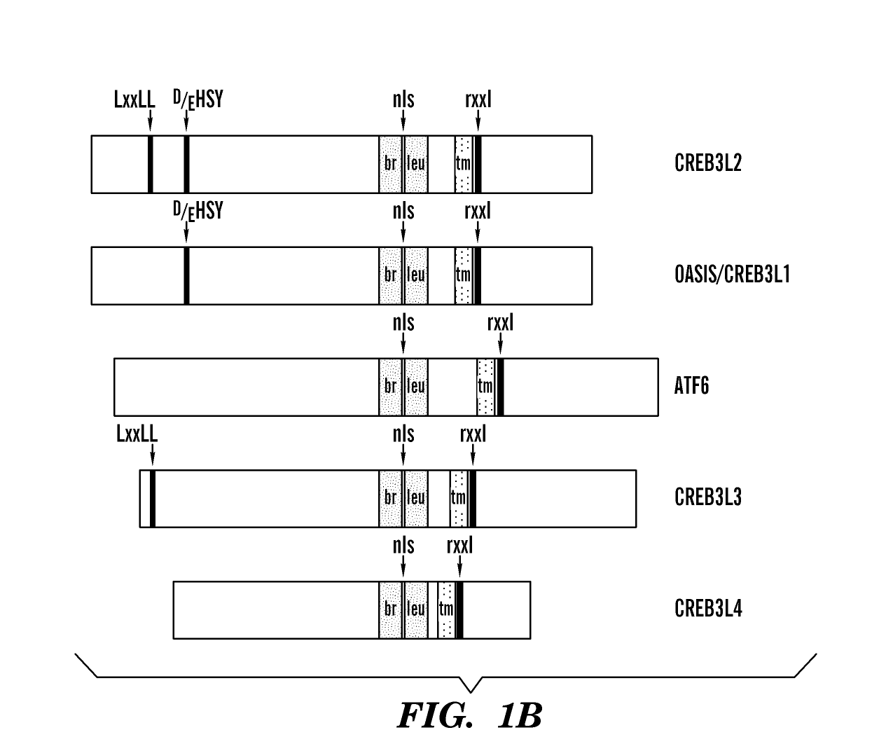

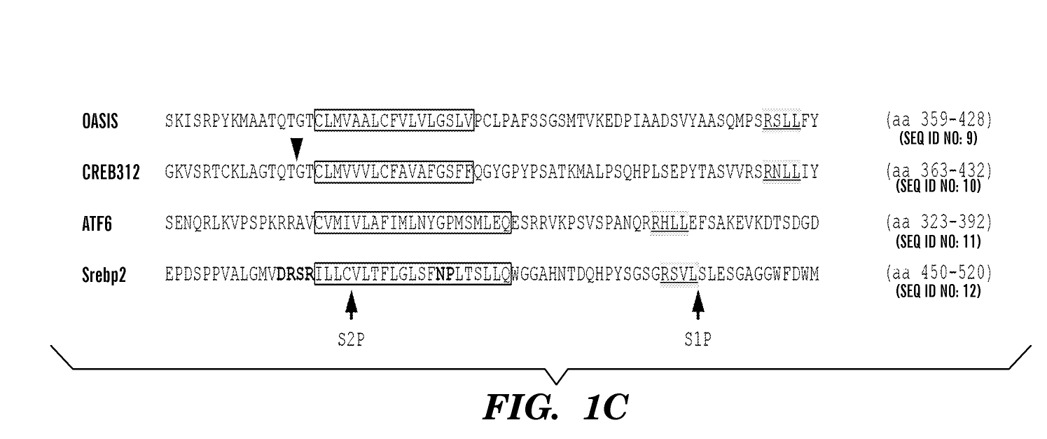

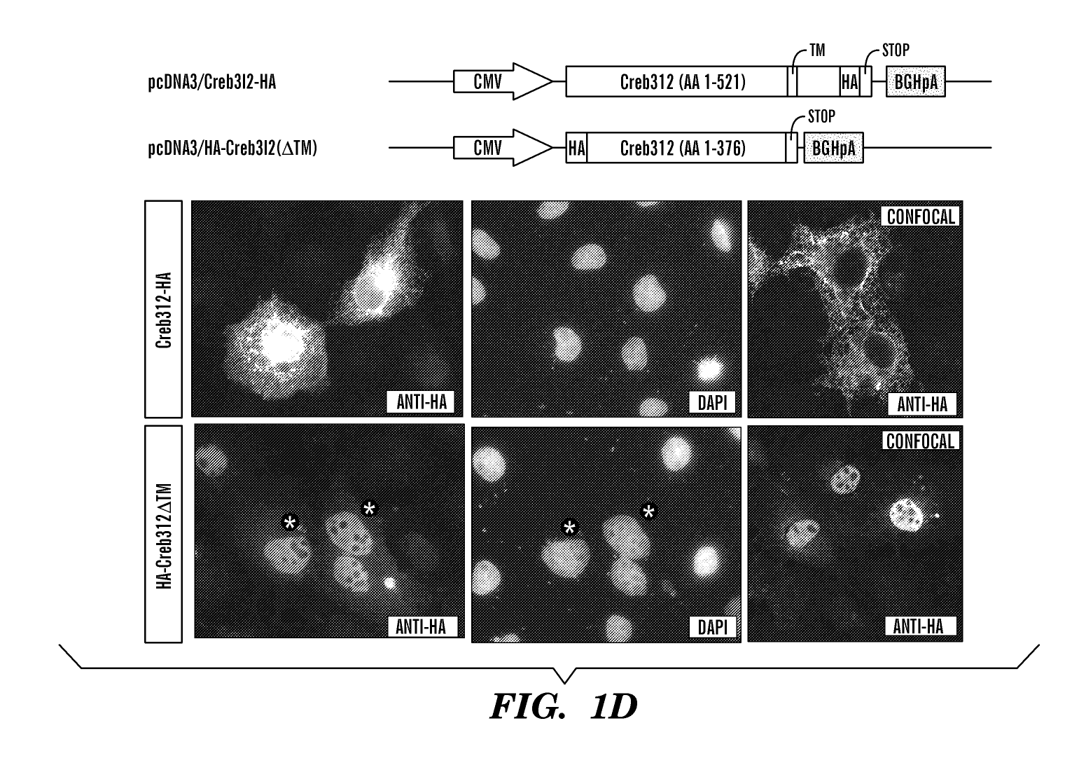

Creb312 is an ER-localized transmembrane protein that is proteolyically processed (i.e. cleaved) into a transcriptionally active nuclear form. The full length Creb312 protein (corresponding to SEQ ID NO: 2) comprises a N-terminal part containing the characteristic DNA-binding basic region, a nuclear localization signal and the leucine zipper region facing the cytosol, a transmembrane domain as well as the C-terminal part facing the ER lumen (see FIG. 1B) and is activated by proteolytic cleavage by regulated intramembrane proteolysis (RIP) by two proteases, the Site-1 protease (SIP) and the Site-2 protease (S2P). Cleavage by the Site-1 protease (SIP) and the Site-2 protease (S2P) releases the N-terminal portion of Creb312 (amino acids residues 1-376 of SEQ ID NO: 2) into the cytosol which translocated to the nucleus and functions as a transcription factor to modulate the transcription of downstream target genes. The human Creb312 polypeptide (SEQ ID NO: 2) is cleaved by SP-2 between amino acid residues 376 and 377, and has a transmembrane domain between amino acids 379-395 and a SP-1 recognition sequence at amino acid residues 427-432, and is cleaved by SP-1 between amino acid residues of 432 and 433 of SEQ ID NO: 2.

Therefore, in some embodiments, an agent useful to promote proliferation of cardiomyocytes or promote survival of cells to mechanical stress as those the terms are used herein is protein or polypeptide of N-terminal fragment of Creb312 or a portion thereof. In some embodiments, such proteins or polypeptides of N-terminal fragment of Creb312 can be administered to a subject in need thereof.

In some embodiments, the agent which is a protein or polypeptide of the N-terminal fragment of Creb312 corresponds to SEQ ID NO: 3 or a functional fragment thereof. The cleavage sites of S1T and S2P on Creb312 are known, as is the location of the transmembrane domain (see FIG. 1C). Therefore, in some embodiments, the N-terminal fragment of Creb312 (also referred to herein as the nuclear form of Creb312) corresponds to SEQ ID NO: 3 or a functional fragment thereof. In alternative embodiments, the N-terminal portion of Creb312 may also include polypeptide chains that start at amino acid No: 1 of SEQ ID NO: 2 and end at any amino acid that is part of the transmembrane domain of SEQ ID NO: 2. That is, the N-terminal portion of Creb312 can be any polypeptide starting at amino acid No: 1 of SEQ ID NO: 2 and end at any amino acid that is part of the transmembrane domain of SEQ ID NO: 2, such as a polypeptide starting at amino acid No: 1 (of SEQ ID NO: 2) and ending from amino acid 379 to 395 of SEQ ID NO: 2. In another embodiment, the N-terminal portion of Creb312 can be any N-terminal portion of Creb312 beginning from amino acid No: 1 of SEQ ID NO: 2 and ending at any amino acid between the enzymatic cleavage sites of SP1 or SP2. For example, the N-terminal portion of Creb312 can be a polypeptide beginning at amino acid No: 1 of SEQ ID NO: 2 and ending between amino acids 376 and 433 inclusive. A nucleic acid sequence encoding the desired N-terminal Creb312 polypeptide can be prepared by recombinant techniques commonly known by one of ordinary skill in the art. In some embodiments, derivatives and functional fragments of the desired N-terminal portion of Creb312 polypeptide can be prepared by techniques commonly known by one of ordinary skill in the art, such as deletions, additions, conservative amino acid substitution, or other manipulations that produce a molecule that maintains the property of promoting proliferation of cardiomyocytes and/or at promoting survival of cells to mechanical stress as the term is used herein, can be administered to a subject in need thereof.

It is well known by persons of ordinary skill in the art that derivatives of natural proteins often retain a biological activity similar to that of the naturally occurring protein. For example, Gayle and coworkers conducted extensive mutational analysis of human cytokine IL-1a. Gayle et al. J. Biol. Chem. 268: 22105-22111 (1993). They used random mutagenesis to generate over 3,500 individual IL-1a mutants that averaged 2.5 amino acid changes per variant over the entire length of the molecule. Multiple mutations were examined at every possible amino acid position. The investigators found that most of the molecule could be altered with little effect on either binding or biological activity. In fact, only, 23 unique amino acid sequences, out of more than 3,500 nucleotide sequences examined, produced a protein that significantly differed in activity from that of the wild-type.

Guidance concerning how to make phenotypically silent amino acid substitutions is provided in Bowie et al., “Deciphering the Message in Protein Sequences: Tolerance to Amino Acid Substitutions, “Science 247: 1306-1310 (1990). The authors indicate that there are two main strategies for studying the tolerance of an amino acid sequence to change. The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection are likely positions that are not critical for protein function. Thus, positions that tolerate amino acid substitution could be modified while still maintaining biological activity of the protein.

The second strategy uses genetic engineering to introduce amino acid changes at specific positions of a cloned gene to identify regions critical for protein function. For example, site directed mutagenesis by alanine-scanning mutagenesis (introduction of single alanine mutations at every residue in the molecule) may be used. Cunningham and Wells, Science 244: 1081-1085 (1989). Bowie et al., Science 247: 1306-1310 (1990), indicate that these two strategies have shown that proteins are surprisingly tolerant of amino acid substitutions. The authors further disclose which amino acid changes are likely to be permissive at certain amino acid positions in the protein. For example, most buried (within the tertiary structure of the protein) amino acid residues require nonpolar side chains, whereas few features of surface side chains are generally conserved.

In general, amino acid substitutions made in order to prepare derivatives or variants of the nuclear form of Creb312 (N-terminal Creb312) polypeptide are accomplished by selecting substitutions that do not differ significantly in their effect on maintaining, for example, (a) the structure of the nuclear form of Creb312 peptide backbone in the area of the substitution, (b) the charge or hydrophobicity of nuclear form of Creb312 (N-terminal Creb312) polypeptide at the target site, or (c) the bulk of a side chain. Naturally occurring residues are divided into groups based on common side-chain properties, for example: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic; trp, tyr, phe. Therefore, conservative amino acid substitutions include, for example, substitution of an aspartic acid residue by a glutamic acid residue because both are acidic amino acids. Similarly, the following examples show acceptable conservative substitutions: lysine/arginine/histidine as basic amino acids; leucine/isoleucine, methionine/valine, alanine/valine as hydrophobic amino acids; serine/glycine/alanine/threonine as hydrophilic amino acids.

Conservative amino acid substitutions also include groupings based on side chains. For example, substitutions include substitutions among amino acids that belong to a group having aliphatic side chains, such as glycine, alanine, valine, leucine, and isoleucine. Similarly, substitutions among a group of amino acids having aliphatic-hydroxyl side chains include substitutions between serine and threonine; substitutions among a group of amino acids of amide-containing side chains include substitutions between asparagine and glutamine; substitutions among a group of amino acids having aromatic side chains include substitutions between phenylalanine, tyrosine, and tryptophan; substitutions among a group of amino acids having basic side chains include substitutions between lysine, arginine, and histidine; and substitutions among a group of amino acids having sulfur-containing side chains include substitutions between cysteine and methionine. Therefore, one skilled in the art can predict based on these guidelines that replacement of a leucine with an isoleucine or valine, an aspartate with a glutamate, a threonine with a serine, or a similar replacement of an amino acid with a structurally related amino acid will not have a major effect on the properties of the resulting nuclear form of Creb312 (N-terminal Creb312) polypeptide derivative.

Additionally, computer programs that predict the effect of a specific amino acid substitution or mutation on the structure of the protein are available. PCT Patent Application No. WO 02/543063 (which is incorporated in its entirety herein by reference) discloses one such method and also reviews the state of the art in this field. Whether any amino acid change (deletion, addition, substitution, or a combination thereof) results in a functional nuclear form of Creb312 polypeptide derivative can readily be determined by assaying the cell-death prevention activity of the derivative in response to mechanical stress, by the method disclosed in the examples, or alternatively by the ability to promote proliferation of cardiomyocytes.

In addition to conservative amino acid substitutions, functional nuclear form of Creb312 polypeptide derivatives used in the instant invention include (i) substitutions with one or more non-conserved amino acid residues, where the substituted amino acid residue may be a chemically modified amino acid (e.g., by methylation, acylation, etc.) that can or can not be encoded by the genetic code, (ii) substitutions with one or more amino acid residues having a substituent group, (iii) fusion of nuclear form of Creb312 polypeptide with another compound, such as a compound to increase the stability and/or solubility of nuclear form of Creb312 polypeptide (for example, polyethylene glycol), or (iv) fusion of the nuclear form of Creb312 polypeptide with additional amino acids. Examples of preparation of derivatives following these guidelines can be found in, for example, U.S. Pat. No. 5,876,969, EP Patent 0 413 622, and U.S. Pat. No. 5,766,883, which are incorporated herein in their entirety by reference.

Such functional derivatives of nuclear form of Creb312 polypeptides may be administered directly to a cell or subject instead of administering a nucleic acid encoding for a functional derivative of nuclear form of Creb312.

Functional derivatives of nuclear form of Creb312 polypeptide that contain deletions or additions of amino acid residues may be produced by using known methods of protein engineering and recombinant DNA technology. For instance, one or more amino acids can be deleted or added from either the N-terminus or C-terminus of the nuclear form of Creb312 (i.e. to either the N- and/or C-terminus SEQ ID NO: 3) without substantial loss of biological function as compared to the a polypeptide corresponding to SEQ ID NO: 3. For example, Ron and coworkers reported variant keratinocyte growth factor (KGF) proteins having heparin binding activity even after deleting 3,8, or 27 amino-terminal amino acid residues. Ron et al. J. Biol. Chem. 268: 2984-2988 (1993). Similarly, interferon gamma exhibited up to ten times higher activity after deleting 8-10 amino acid residues from the carboxy terminus of the protein. Dobeli et al., J. Biotechnol. 7: 199-216 (1988).

Accordingly, functional derivatives of the nuclear form of Creb312 may be prepared by modification of the amino acids in the nuclear form of Creb312 (i.e. SEQ ID NO: 3) or in a derivative of the nuclear form of Creb312. Modifications may occur anywhere in the nuclear form of Creb312 polypeptide sequence or its functional derivative polypeptide, including the peptide backbone, the amino acid side-chains and the amino or carboxyl termini. Modifications may include, for example, acetylation, acylation, ADP-ribosylation, amidation, covalent attachment of other functional moiety, covalent attachment of a lipid or lipid derivative, covalent attachment of phosphotidylinositol, cross-linking, cyclization, disulfide bond formation, demethylation, formation of covalent cross-links, formylation, gamma-carboxylation, glycosylation, glycophosphatidylinositol (GPI) anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, E. Creighton Proteins-Structure and Molecular Properties, 2nd Ed., W. H. Freeman and Company, New York (1993); B. C. Johnson, Post Translational Covalent Modification of Proteins, Academic Press, New York, (1983); Seifter et al., Meth. Enzymol. 182: 626-646 (1990); Rattan et al., Ann. N.Y. Acad. Sci. 663: 48-62 (1992). Preparation of these modified derivatives may, for example, be useful if direct administration of the derivative, rather than administration of a nucleic acid encoding the derivative, is contemplated.

Because of the degeneracy of genetic code, the skilled artisan may prepare more than one nucleic acid sequence that encodes the nuclear form of Creb312 polypeptide or one of its functional derivative polypeptides. Administration of nucleic acids obtained in this manner is also encompassed by the instant invention.

Use of natural and non-natural allelic variations of the nuclear form of Creb312 is also contemplated as being part of the invention. These allelic variants can vary at either the polynucleotide and/or polypeptide level. Non-naturally occurring variants may be produced by mutagenesis techniques or by direct synthesis. Therefore, the present invention is also directed to proteins containing polypeptides at least 80%, 85%, 90%, 95%, 96%, 97%, 98% or 99% identical to the nuclear form of Creb312 polypeptide sequences corresponding to SEQ ID NO: 3. The methods of the invention may also utilize nucleic acid molecules comprising, or alternatively, consisting of, a polynucleotide sequence at least 70%, 75%, 80%, 85%, 90%, 95%, 96%, 97%, 98% or 99% identical to the polynucleotide sequence encoding the nuclear form of Creb312. Whether any particular nucleic acid molecule or polypeptide is at least 80%, 85%, 90%, 95%, 96%, 97%, 98% or 99% identical to an Creb312 sequence, or a portion of a nucleic acid sequence encoding Creb312 can be determined conventionally using known computer programs. See, e.g., WO 01/54474 which is incorporated herein by reference.

Nucleic acids encoding these homologous polynucleotide sequences can be isolated by hybridization under different conditions. It is well known in the art that the degree of stringency (high, intermediate, low, etc.) depends on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature of the probe to about 20° C. to 25° C. below the melting temperature). Functionally, maximum stringency conditions may be used to identify nucleic acid sequences having strict identity or near-strict identity with the hybridization probe; while high stringency conditions may be used to identify nucleic acid sequences having about 80% or more sequence identity with the probe. Persons skilled in the art will appreciate that one or more factors be may be varied to generate conditions of stringency that may be different from each other in temperature and ionic strength composition but that are equivalent to in terms of the results desired. One general guide for nucleic acid hybridization is Tijssen, “Laboratory Techniques in Biochemistry and Molecular Biology Elsevier, vol. 24, Hybridization with Nucleic Acid Probes, Part I Overview of principles of hybridization and the strategy of nucleic acid assays (1993). Whether any homologous polynucleotide sequence encodes a functional nuclear form of Creb312 derivative can readily be determined as discussed above, by assaying the polypeptide product of the nucleic acid sequence to prevent cell-death in response to mechanical stress as disclosed in the method section in the examples, or alternatively by the ability of the polypeptide product of the nucleic acid sequence to promote proliferation of cardiomyocytes.

As disclosed in the Examples and in FIG. 7, the nuclear form of Creb312 acts as a transcription factor and binds to part of the BNP promoter region (or the BNP response element), thereby inducing the expression of BNP-responsive genes. If other transcription factors are required for activation of the BNP response element, them in one embodiment, the method for promoting cell survival in response to mechanical stress or promoting proliferation of cardiomyocytes can optionally additionally comprise introducing a vector comprising a nucleic acid sequence encoding a basal transcription factor polypeptide that binds to the BNP response element.

Furthermore, a method for promoting cell survival in response to mechanical stress and/or promoting proliferation of cardiomyocytes can comprise any known method in the art that induces the cleavage of endogenous full length Creb312 (i.e. SEQ ID NO: 2) to the nuclear form of Creb312. In alternative embodiments, a method for promoting cell survival in response to mechanical stress and/or promoting proliferation of cardiomyocytes can comprise any known method in the art that induces the expression of an endogenous nuclear form of Creb312, or homologue or variant thereof.

In mammalian cells, activation of ATF6 has been shown to involve the activation of the transmembrane kinase Ire1, of which two isoforms have been identified (activation of Ire1 is induced by the accumulation of unfolded proteins in the ER). van Laar et al., Current Protein and Peptide Science 2: 169-190 (2001) (which is incorporated herein in its entirety by reference). Activation of Ire1 involves dimerization and autophosphorylation, after which presenilin 1 and 2 cleave the kinase-nuclease domain of Ire1. This cleavage occurs in a RIP-like fashion analogous to the release of the N-terminus of ATF6 after cleavage by SP1 and/or SP2. Accordingly, Ire1 could be useful in the methods of the present invention for the activation of full length Creb312 to the nuclear form of Creb312.

In some embodiments, preparation of a N-terminal Creb312 polypeptide, such as a soluble N-terminal Creb312 polypeptide for use in the methods and compositions as disclosed herein can be produced by any means known by one of ordinary skill in the art, for example in cultured cells expressing the recombinant nuclear form of Creb312 protein. In some embodiments, the N-terminal Creb312 polypeptide or a functional portion or fragment thereof can be administered by any means known by a skilled artisan, for example direct administration of the protein, or administration of cells expressing the recombinant nuclear form of Creb312 protein. Administration can be via any route, for example but not limited to administration intravenously. This approach rapidly delivers the protein throughout the system and maximizes the change that the protein is intact when delivered. In alternative embodiments, the polypeptide can be administered directly to location of the heart by any means, or other routes of therapeutic protein administration are contemplated such as inhalation.

In some embodiments, cells used for implantation in a cell-based therapy for a cardiovascular disease or disorder can be contacted with agents to increase the N-terminal Creb312 in such cells, i.e. for example, such cell for transplantation or implantation into a host subject can be genetically modified to express N-terminal Creb312. In such an embodiment, cells to be implanted for cell-based therapy for cardiovascular disease or a disorder can be modified (e.g., by homologous recombination) to increase the expression of such an agent, for example by replacing, in whole or in part, the naturally occurring promoter with all or part of a heterologous promoter so that the cells express the N-terminal Creb312 or a functional portion thereof. The heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems, which are incorporated herein in their entirety by reference.

In alternative embodiments, cells to be transplanted into a host subject can be modified ex vivo using conventional gene therapy techniques to introduce nucleic acid encoding Creb312(N) polypeptide to result in an increase the expression of a Creb312(N) polypeptide in the cell. The nucleic acid can be introduced into the cell using any variety of commonly known methods, for example, using vectors, such as viral vectors to tranduce the cell and increase the expression of the Creb312(N) polypeptide in the cell, as discussed herein under the section entitled “Vectors and Expression of agents which increase the nuclear form of Creb312, or expression of nuclear form of human Creb312”.

In some embodiments, the heterologous promoter allows controlled expression of the N-terminal Creb312, such as for example, and drug or stress inducible promoter, such as a Tet-inducible system and the like. For example, cells can be engineered to express an endogenous gene comprising the agent, or the N-terminal Creb312 under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al, which are incorporated herein in their entirety by reference.

In some embodiments, the agent can be prepared by culturing transformed host cells under culture conditions suitable to express the agent, such as N-terminal Creb312. The resulting expressed agent can then be purified from such culture (i.e., from culture medium or cell extracts) using known purification processes, such as gel filtration and ion exchange chromatography. The purification of the agent, or alternatively, the N-terminal Creb312 protein, or functional portion thereof can also include one or more column steps over such affinity resins as concanavalin A-agarose, HEPARIN-TOYOPEARL™ or Cibacrom blue 3GA Sepharose; one or more steps involving hydrophobic interaction chromatography using such resins as phenyl ether, butyl ether, or propyl ether; immunoaffinity chromatography, or complementary cDNA affinity chromatography. de an affinity column containing agents which will bind to the protein.

In one embodiment, the nucleic acid agents useful herein can be obtained synthetically, for example, by chemically synthesizing a nucleic acid by any method of synthesis known to the skilled artisan. The synthesized nucleic acid agents can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle, which is incorporated herein in its entirety by reference).

In some embodiments, an agent that increases the nuclear form of Creb312 are proteins and/or peptides, or fragments of agents which increase nuclear Creb312, for example, but are not limited to mutated proteins; therapeutic proteins and recombinant proteins. Proteins and peptides inhibitors can also include for example mutated proteins, genetically modified proteins, peptides, synthetic peptides, recombinant proteins, chimeric proteins, antibodies, humanized proteins, humanized antibodies, chimeric antibodies, modified proteins and fragments thereof. In some embodiments, an agent which increases nuclear Creb312 is a dominant negative variant of an inhibitor of Creb312, for example a non-functional variant of a protein which inhibits the cleavage of full length Creb312 to the nuclear form of Creb312.

In some embodiments, a nuclear Creb312 polypeptide or a portion thereof functional to induce gene expression of BNP or to at least increase the survival of a cell in response to mechanical stretch stress, as that term is defined herein, or to increase the proliferation of cardiomyocyte, can be directly administered to a subject in need thereof.

In one approach, a nuclear form of Creb312 (corresponding to SEQ ID NO: 3) can be produced, for example, in cultured cells bearing a recombinant nuclear form of Creb312 expression vector can be administered to the individual. The nuclear form of Creb312 polypeptide or portion thereof can be administered intravenously. This approach rapidly delivers the protein throughout the system and maximizes the chance that the protein is intact when delivered. Alternatively, other routes of therapeutic protein administration are contemplated, such as by inhalation. Technologies for the administration of agents, including protein agents, as aerosols are well known and continue to advance. Alternatively, the polypeptide agent can be formulated in liposomes for topical delivery. Further contemplated are, for example, transdermal administration, and rectal or vaginal administration. Further options for the delivery of nuclear form of Creb312 polypeptides as described herein are discussed in the section “Pharmaceutical Compositions” herein below. Dosage ranges will vary, depending upon the individual, the degree of disease severity, and the specific polypeptide administered, but can be readily selected and adjusted by the administering clinician. An exemplary dose range is approximately 0.01 μg/kg to 1 mg/kg per dose, with doses administered, for example, once a week, once every three days, once every other day, or even daily. Initial doses can be greater, to establish an effect, and then reduced to a maintenance level thereafter.

For the treatment of a cardiac disease, or cardiovascular disorder or injury as disclosed herein, an alternative to administration of polypeptide directly is to use a cell based delivery administration, where a cell, or population of cells are ex vivo modified to express a Creb312(N) polypeptide are administered to a subject, and where the cell or population of cells secrete the Creb312(N) polypeptide. This approach provides potentially long-term delivery of the agent that also provides a steadier level than, for example, repeated bolus administration by injection. While it is not absolutely necessary, in a preferred embodiment, the cells can be autologous cells, taken from the patient to be treated, transfected or otherwise transduced with a transgene encoding the therapeutic polypeptide, and then re-introduced to the patient. Vectors for transduction of an nuclear form of Creb312-encoding sequence are well known in the art. While overexpression using a strong non-specific promoter, such as a CMV promoter, can be used, it can be helpful to include a tissue- or cell-type-specific promoter on the expression construct—for example, the use of a cardiac muscle-specific promoter or other cell-type-specific promoter can be advantageous, depending upon what cell type is used as a host.

Further, treatment can include the administration of viral vectors that drive the expression of nuclear form of Creb312 polypeptides in infected host cells. Viral vectors are well known to those skilled in the art and discussed further herein below. Other avenues for the administration of nuclear form of Creb312 polypeptides for therapy are discussed herein below—these can be applied to the treatment of cardiovascular diseases or disorders responsive to nuclear form of Creb312 polypeptides and to the cardioprotective aspect of the disclosed invention, among others.

2) Proteases that cleave Creb312 at SP1 and/or SP2 sites

In some embodiments, a nuclear form of Creb312 (corresponding to SEQ ID NO: 3) can be produced, for example, by post-translational processing of full length Creb312 (of SEQ ID NO: 2). Post-translational processing can comprise cleavage of full length Creb312 by S1T and/or S2P. As discussed above, full length Creb312 (SEQ ID NO: 2) is cleaved to produce the active nuclear form of Creb312 (SEQ ID NO: 3) by proteolytic cleavage by regulated intramembrane proteolysis (RIP) by two proteases, the Site-1 protease (SIP) and the Site-2 protease (S2P). Cleavage by the Site-1 protease (SIP) and the Site-2 protease (S2P) releases the N-terminal portion of Creb312 (amino acids residues 1-376 of SEQ ID NO: 2) into the cytosol which translocated to the nucleus and functions as a transcription factor to modulate the transcription of downstream target genes. The human Creb312 polypeptide (SEQ ID NO: 2) is cleaved by SP-2 between amino acid residues 376 and 377, and has a transmembrane domain between amino acids 379-395 and a SP-1 recognition sequence at amino acid residues 427-432, and is cleaved by SP-1 between amino acid residues of 432 and 433 of SEQ ID NO: 2.

Accordingly, the nuclear form of Creb312 (corresponding to SEQ ID NO: 3) is the product of cleavage of full length Creb312 by S1T and/or S2P. In some embodiments, an agent useful to promote proliferation of cardiomyocytes or promote survival of cells to mechanical stress as disclosed herein is any agent or stimuli which induces the cleavage of full length Creb312 to produce the nuclear form of Creb312 of SEQ ID NO: 2. Methods to identify agents which cleave full length Creb312 to the nuclear form of Creb312 are known by persons of ordinary skill in the art. One such method is disclosed in US patent Application US2007/0111258, which is incorporated herein in its entirety by reference, where one can substitute CREBH with Creb312, thereby enabling identification of agents which cleave Creb312 in place of CREBH. Examples of such agents are demonstrated in the Examples, and include but are not limited to dithiothreitol (DTT), Tm and thapsogargin (Tg) and Brefeldin A (BFA).

In some embodiments, an agent, such as a protease which cleavages the full length Creb312 to produce the nuclear form of Creb312 of SEQ ID NO: 2 is useful in the methods and compositions to promote proliferation of cardiomyocytes or promote survival of cells to mechanical stress as disclosed herein. Stated another way, any protease which results in the cleavage of full length Creb312 to the active nuclear form is useful in the methods and compositions as disclosed herein. In some instances, proteases, in particular serine proteases exist as inactive precursors and preproenzymes and contain a leader or activation peptide sequence 3′ of the signal peptide. Cleavage of the leader sequence or pre-sequence of the proenzyme protease activates the protease. The leader sequence or pro-sequence varies in different serine proteases; typically, this activation peptide may be 2-12 amino acids in length. This sequence varies in different serine proteases according to the biochemical pathway and/or its substrate (Zunino et al. (1988) Biochimica et. Biophysica Acta 967:331 340; Sayers, et al. (1992) J. Immunology 148:292 300). Other features that distinguish various serine proteases are the presence or absence of N-linked glycosylation sites that provide membrane anchors, the number and distribution of cysteine residues that determine the secondary structure of the serine protease and the sequence of a substrate binding sites such as S′. The S′ substrate binding region is defined by residues extending from approximately +17 to +29 relative to the N-terminal (+1). Differences in this region of the molecules are believed to determine serine protease substrate specificities (Zunino et al, supra). Accordingly, in one embodiment, an agent useful to promote proliferation of cardiomyocytes or promote survival of cells to mechanical stress as disclosed herein can be a protease which cleaves the full length Creb312 to produce the nuclear form of Creb312 of SEQ ID NO: 2 is an inactive precursor protease. In some embodiments, an agent is S1P and/or S2P or a MT-SP1, as disclosed in U.S. Pat. No. 7,030,231 which is incorporated herein in its entirety by reference. Proteases or biologically active protease polypeptide fragments (i.e. the proteolytic domain of a protease) for use in the methods and compositions as disclosed herein can be produced by recombinant expression systems by a person of ordinary skill in the art, and as disclosed in U.S. Pat. No. 7,030,231 which is incorporated herein in its entirety by reference.

In some embodiments, an agent is a pro form of SIT (also known in the art as “S1P-A”) which is the inactive precursor of SIT (see Cheng et al., J Biol Chem, 1999; 274; 22805-22812, which is incorporated herein in its entirety by reference). S1P-1 proenzyme undergoes two further cleavages to generate S1P-B and S1P-C active enzymes (Espenshade et al., J Biol Chem, 1999; 274; 22795-804, which is incorporated herein in its entirety by reference). Accordingly, in another embodiment, an agent is S1P-B (residues 137-1052) or S1P-C (residues 187-1052) of SIT protease. The polypeptide sequence for SIT is disclosed as SEQ ID NO: 1 in U.S. Pat. No. 6,322,962, which is incorporated herein in its entity by reference. Methods to identify agents which inhibit SIT are also disclosed in U.S. Pat. No. 6,322,962, however, one of ordinary skill in the art can use the methods taught in U.S. Pat. No. 6,322,962 to identify agents which increase the activity or expression of SIP, and thus are useful as an agent herein for the methods and compositions to promote proliferation of cardiomyocytes or promote survival of cells to mechanical stress.

Similarly, International Application WO/2004/078934, which is incorporated herein in its entirety by reference, discloses S2P (also known as “Membrane-bound transcription factor site 2 protease” or “MBTFP” in the art) and teaches methods to identify agents which modify S2P (i.e. inhibit the activity or expression or increase the activity or expression of S2P). Accordingly, one of ordinary skill in the art can use the methods taught in WO/2004/078934 to identify agents which increase the activity or expression of S2P, and thus are useful as an agent herein for the methods and compositions to promote proliferation of cardiomyocytes or promote survival of cells to mechanical stress. Furthermore, orthologues or homologues of S1T and/or S2P are useful as agents in the methods and compositions as disclosed herein, and are disclosed in US Patent Application 2004/0203105, which is incorporated herein in its entirety by reference.

### 3) ILK Polypeptide

In another embodiment, proteolytic cleavage of full length Creb312 can be activated by the polypeptide integrin-linked kinase (ILK) polypeptide or an agent which activates the ILK polypeptide. As demonstrated herein in Example 4, the inventors demonstrated that inhibition of ILK via RNAi reduced activation of full length Creb312 to the nuclear form of Creb312.

Without wishing to be bound by theory, ILK is a serine-theronine kinase which is implicated in cell-to-extracellular matrix interaction, growth factor signaling, cell survival, cell migration, invasion, anchorage-independent growth and angiogenesis. Increased expression of ILK has been associated with the progression of several tumor types. Integrin-1 inked kinase binds to the cytoplasmic domains of β1, β2 and β3-integrin subunits. ILK serves as a molecular scaffold at sites of integrin-mediated adhesion, anchoring cytoskeletal actin and nucleating a supramolecular complex comprised minimally of ILK, PINCH and β-parvin. In addition to its structural role, ILK is a signaling kinase coordinating cues from the ECM in a phosphoinositide 3′-kinase (PBK)-dependent manner following distinct signal inputs from integrins and growth factor receptor tyrosine kinases. ILK lies upstream of kinases shown in experimental models to modulate hypertrophy, and is required for phosphorylation of protein kinase B (Akt/PKB) at Ser473 and GSK3β at Ser9. Rho-family guanine triphosphatases (GTPases, or G-proteins), including RhoA, Cdc42, and Rac1, modulate signal transduction pathways regulating actin cytoskeletal dynamics in response to matrix interaction with integrin and other cell surface receptors. Both RhoA and Rac1 have been shown to modulate cardiac hypertrophy. ECM adhesion stimulates the increased association of activated, GTP-bound Rac1 with the plasma membrane, suggesting a role for ILK in promoting membrane targeting of activated Rac1. ILK may also activate Rac1 through regulated interaction of the Rac1/Cdc42 specific guanine-nucleotide exchange factor (GEF), ARHGEF6/α-PIX, with β-parvin, an ILK-binding adaptor, as occurs during cell spreading on fibronectin. The ILK nucleic acid sequences, polypeptide and anti-ILK antibodies are disclosed in U.S. Pat. Nos. 6,369,205; 6,013,782; 6,001,622; 6,338,958; 6,699,983; 7,189,802 and related foreign equivalent patent applications, which are all incorporated herein in their entirety by reference. Furthermore, U.S. Pat. Nos. 6,013,782 and 6,699,983 are directed toward methods for isolating ILK genes and suggest that modulation of the gene activity in vivo might be useful for prophylactic and therapeutic purposes, but these applications fails to teach or suggest any perceived benefit relative to over or under expression of ILK with respect to increasing the nuclear form of Creb312, or for use to increase cardiomyocyte proliferation, cardiac hypertrophy or post MI (myocardial ischemia) cardiac remodeling.

In one embodiment the present invention relates to agents which can be administered to cells or a subject to induce the cleavage of full length Creb312 to a nuclear (active) form of Creb312 to promote the survival of cell in response to mechanical stress, and/or promote proliferation of cardiomyocytes. As disclosed in the Examples, ILK promotes the proteolytic cleavage of full length Creb312 to the nuclear form, and inhibition of ILK via RNAi reduced activation of full length Creb312 to the nuclear form of Creb312. Previous uses of ILK have been used to look at the role of ILK to enhance post-infarct remodeling in mice. Overexpression of ILK polypeptide has only been shown to increase the cardiomyocyte size (i.e. hypertrophy of cardiomyocytes) but have not demonstrated to induce or increase in the proliferation (i.e. the number) of cardiomyocytes. For example, the International Application WO2007137414, which is incorporated herein in its entirety by reference, by Coles et al., discussed that ILK protein expression is increased in the hypertrophic human ventricle, and that ILK expression levels correlate with increased GTP loading, or activation, of the small G-protein, Rac1. Transgenic mice with cardiac-specific activation of ILK signaling have shown to exhibit compensated LV hypertrophy and ILK over-expressing mice lines exhibit higher levels of activated Rac1 and Cdc42, in association with activation of p38 mitogen-activated protein (p38MAPK) and ERK 1/2 kinase cascades. Additionally, Coles et al., also discloses that increased ILK expression can enhance post-infarct remodeling in mice. However, unlike the present invention, Coles et al., teaches that ILK activity regulates cardiomyocyte size and specifically teaches against the present invention that ILK does not regulate cardiomyocyte proliferation. Furthermore, Coles et al., demonstrate a protective effect of ILK to reduce the scar area after myocardial infarct. However, the inventors of Coles et al., attribute this to increased hypertrophic response. Unlike the present invention, Coles et al., does not teach or make any suggestion that the ILK polypeptide can promote the survival of cells, nor does it teach survival of cardiomyocytes in response to mechanical stress, or stretch mechanical stress. Importantly, Coles et al., also fails to teach or suggest any perceived benefit of ILK for increasing the proteolytic cleavage of full length Creb312 (Creb312FL) to the nuclear form of Creb312 (Creb312(N)) to result in increased cell survival in response to mechanical stress such as stretch mechanical stress or for the increase in the proliferation of cardiomyocytes, such as postnatal cardiomyocytes.

Accordingly, another agent useful in the methods and compositions as disclosed herein for promoting survival of cells in response to stress or for increasing the propagation of a population of cardiomyocytes is an ILK polypeptide. In some embodiments, an ILK polypeptide can be administered to the cells in need of survival in response to stress, such as mechanical stress, or to increase proliferation. In another embodiments, one can overexpress a ILK polypeptide in a cell in need of survival in response to stress, such as mechanical stress, or to increase proliferation. Any means to overexpress the ILK polypeptide known by a person skilled in the art can be used, such as is taught above, and in the disclosure of International Application WO2007/137414, which is incorporated herein in its entirety by reference.

### 4) Antibodies

In some embodiments, an agent which increases nuclear form of Creb312 useful in the methods and compositions of the present invention include, for example, antibodies, including monoclonal, chimeric humanized, and recombinant antibodies and antigen-binding fragments thereof. An antibody useful in the methods and compositions as disclosed herein can include any antibody which binds to and inhibits a protein, which when not bound by the antibody, functions to inhibit the formation of the nuclear form of Creb312 from full length Creb312. In alternative embodiments, an antibody useful as an agent which increases nuclear form of Creb312 useful in the methods and compositions can be any antibody which binds to and activates a protein, which when not bound by the antibody, functions to activate the formation of the nuclear form of Creb312 from full length Creb312. By way of an example only, such antibodies include activating antibodies which bind to an activate proteases, such as site-1 proteases and/or site-2 proteases which when active promote the cleavage of full length Creb312 to the nuclear form of Creb312.

Antibodies useful in the present invention can readily raised in animals such as rabbits or mice by immunization with the antigen. Immunized mice are particularly useful for providing sources of B cells for the manufacture of hybridomas, which in turn are cultured to produce large quantities of monoclonal antibodies. In one embodiment of this invention, an agent which increases nuclear form of Creb312 useful in the methods and compositions of the present invention include, for example, can be an antibody molecule or the epitope-binding moiety of an antibody molecule and the like. Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis.

Useful monoclonal antibodies and fragments can be derived from any species (including humans) or can be formed as chimeric proteins which employ sequences from more than one species. Human monoclonal antibodies or “humanized” murine antibody are also used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction the possibly of adverse immune reactions in a manner similar to that disclosed in European Patent Application No. 0,411,893 A2. A murine monoclonal antibodies can preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

One limitation of scFv molecules is their monovalent interaction with target antigen. One of the easiest methods of improving the binding of a scFv to its target antigen is to increase its functional affinity through the creation of a multimer. Association of identical scFv molecules to form diabodies, triabodies and tetrabodies can comprise a number of identical Fv modules. These reagents are therefore multivalent, but monospecific. The association of two different scFv molecules, each comprising a VH and VL domain derived from different parent Ig will form a fully functional bispecific diabody. A unique application of bispecific scFvs is to bind two sites simultaneously on the same target molecule via two (adjacent) surface epitopes. These reagents gain a significant avidity advantage over a single scFv or Fab fragments. A number of multivalent scFv-based structures has been engineered, including for example, miniantibodies, dimeric miniantibodies, minibodies, (scFv)₂, diabodies and triabodies. These molecules span a range of valence (two to four binding sites), size (50 to 120 kDa), flexibility and ease of production. Single chain Fv antibody fragments (scFvs) are predominantly monomeric when the VH and VL domains are joined by, polypeptide linkers of at least 12 residues. The monomer scFv is thermodynamically stable with linkers of 12 and 25 amino acids length under all conditions. The noncovalent diabody and triabody molecules are easy to engineer and are produced by shortening the peptide linker that connects the variable heavy and variable light chains of a single scFv molecule. The scFv dimers are joined by amphipathic helices that offer a high degree of flexibility and the miniantibody structure can be modified to create a dimeric bispecific (DiBi) miniantibody that contains two miniantibodies (four scFv molecules) connected via a double helix. Gene-fused or disulfide bonded scFv dimers provide an intermediate degree of flexibility and are generated by straightforward cloning techniques adding a C-terminal Gly4Cys sequence. scFv-CH₃ minibodies are comprised of two scFv molecules joined to an IgG CH3 domain either directly (LD minibody) or via a very flexible hinge region (Flex minibody). With a molecular weight of approximately 80 kDa, these divalent constructs are capable of significant binding to antigens. The Flex minibody exhibits impressive tumor localization in mice. Bi- and tri-specific multimers can be formed by association of different scFv molecules. Increase in functional affinity can be reached when Fab or single chain Fv antibody fragments (scFv) fragments are complexed into dimers, trimers or larger aggregates. The most important advantage of multivalent scFvs over monovalent scFv and Fab fragments is the gain in functional binding affinity (avidity) to target antigens. High avidity requires that scFv multimers are capable of binding simultaneously to separate target antigens. The gain in functional affinity for scFv diabodies compared to scFv monomers is significant and is seen primarily in reduced off-rates, which result from multiple binding to two or more target antigens and to rebinding when one Fv dissociates. When such scFv molecules associate into multimers, they can be designed with either high avidity to a single target antigen or with multiple specificities to different target antigens. Multiple binding to antigens is dependent on correct alignment and orientation in the Fv modules. For full avidity in multivalent scFvs target, the antigen binding sites must point towards the same direction. If multiple binding is not sterically possible then apparent gains in functional affinity are likely to be due the effect of increased rebinding, which is dependent on diffusion rates and antigen concentration. Antibodies conjugated with moieties that improve their properties are also contemplated for the instant invention. For example, antibody conjugates with PEG that increases their half-life in vivo can be used for the present invention. Immune libraries are prepared by subjecting the genes encoding variable antibody fragments from the B lymphocytes of naive or immunized animals or patients to PCR amplification. Combinations of oligonucleotides which are specific for immunoglobulin genes or for the immunoglobulin gene families are used. Immunoglobulin germ line genes can be used to prepare semisynthetic antibody repertoires, with the complementarity-determining region of the variable fragments being amplified by PCR using degenerate primers. These single-pot libraries have the advantage that antibody fragments against a large number of antigens can be isolated from one single library. The phage-display technique can be used to increase the affinity of antibody fragments, with new libraries being prepared from already existing antibody fragments by random, codon-based or site-directed mutagenesis, by shuffling the chains of individual domains with those of fragments from naive repertoires or by using bacterial mutator strains.

Alternatively, a SCID-hu mouse, for example the model developed by Genpharm, can be used to produce antibodies, or fragments thereof. In one embodiment, a new type of high avidity binding molecule, termed a “peptabody” has been created by harnessing the effect of multivalent interaction is contemplated. A short peptide ligand was fused via a semirigid hinge region with the coiled-coil assembly domain of the cartilage oligomeric matrix protein, resulting in a pentameric multivalent binding molecule. In a preferred embodiment of this invention, ligands and/or chimeric inhibitors can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. Alternatively, two or more active agents and or inhibitors attached to targeting moieties can be administered, wherein each conjugate includes a targeting moiety, for example, a different antibody. Each antibody is reactive with a different target site epitope (associated with the same or a different target site antigen). The different antibodies with the agents attached accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

### 5) Small Molecules

As disclosed in the Examples, full length endogenous Creb312 is cleaved into the nuclear (active) form in response to ER-stress inducing agents, such as for example brefeldin A (BFA), thapsigargain (TG), DTT and tunicamycin (Tm) treatment as disclosed herein in the examples as well as hypoxia, hormonal stresses such as isoproterenol (ISO), phenylephrine (PE), angliotensin-2 (AT-2), endothelin-1 (ET-1). Accordingly, any agent known by a skilled artisan to (i) induce ER stress, or (ii) induce hormonal stress, or (iii) induce hypoxic stress is useful in the methods and compositions of the present invention to increase the nuclear form of Creb312. In some embodiments and as disclosed in the Examples, an agent which increases the formation of nuclear form of Creb312 can be selected from at least one, or a combination of the following agents; brefeldin A (BFA), thapsigargain (TG), DTT, tunicamycin (Tm), low O₂, isoproterenol (ISO), hypertrophic agonists such as phenylephrine (PE), angiotensin-2 (AT-2) and endothelin-1 (ET-1).

Nucleic Acid Agents which Increase Nuclear Creb312

In some embodiments, the present invention encompasses increasing the nuclear form of Creb312 by introducing into a cell, such as a cardiac progenitor cell and/or cardiomyocyte, a nucleic acid encoding the polypeptide of SEQ ID NO: 3. In some embodiments, the nucleic acid is a nucleic acid analogue or variant thereof, as disclosed herein. Any means to introduce a nucleic acid into a cell can be used, for example, using vectors, such as viral vectors to tranduce the cell and increase the expression of the Creb312(N) polypeptide in the cell, as discussed herein under the section entitled “Vectors and Expression of agents which increase the nuclear form of Creb312, or expression of nuclear form of human Creb312”.

In an alternative embodiment, an agent used to increase the level of nuclear form of Creb312 in a cell is nucleic acid inhibitor which decreases the expression of a protein inhibitor of a protease, for example a nucleic acid which inhibits or inactivates an inhibitor to protease site-1 protease (SIP) and/or site-2 proteases (S2P). Such nucleic acid inhibitors include for example, but not are limited to, RNA interference-inducing (RNAi) molecules, for example but are not limited to siRNA, dsRNA, stRNA, shRNA and modified versions thereof, where the RNA interference molecule silences the gene expression of inhibitors of Creb312(N) or site-1 or site-2 proteases. In some embodiments, the nucleic acid inhibitor of Creb312(N) or site-1 or site-2 proteases is an anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like. In alternative embodiments, the nucleic acid is DNA or RNA, and nucleic acid analogues, for example PNA, pcPNA and LNA. A nucleic acid can be single or double stranded, and can be selected from a group comprising nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribosomes, small inhibitory nucleic acid sequences, for example but are not limited to RNAi, shRNAi, siRNA, micro RNAi (mRNAi), antisense oligonucleotides etc.

In some embodiments single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

RNA interference (RNAi) provides a powerful approach for inhibiting the expression of selected target polypeptides. RNAi uses small interfering RNA (siRNA) duplexes that target the messenger RNA encoding the target polypeptide for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

The term “short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA Apr.; 9(4):493-501, incorporated by reference herein in its entirety).

The target gene or sequence of the RNA interfering agent can be a cellular gene or genomic sequence. An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any skilled artisan using commonly known sequence comparison methods, such as BLAST, as described herein and using default parameters.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotidesmolecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting to increase Creb312(N) expression can be readily designed and tested. Accordingly, siRNAs useful for the methods described herein include siRNA molecules of about 15 to about 40 or about 15 to about 28 nucleotides in length, which are homologous to the target gene to be gene silenced. Preferably, the targeting siRNA molecules have a length of about 19 to about 25 nucleotides. More preferably, the targeting siRNA molecules have a length of about 19, 20, 21, or 22 nucleotides. The targeting siRNA molecules can also comprise a 3′ hydroxyl group. The targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the targeting RNA molecule is double stranded—one strand has a 3′ overhang and the other strand can be blunt-ended or have an overhang. In the embodiment in which the targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′hydroxyl significantly enhances the nuclease resistance of the overhang in tissue culture medium.

siRNA sequences are chosen to maximize the uptake of the antisense (guide) strand of the siRNA into RISC and thereby maximize the ability of RISC to target the target gene mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the target mRNA.

In a preferred embodiment, the siRNA or modified siRNA is delivered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting. In one embodiment, the vector can be a regulatable vector, such as tetracycline inducible vector.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

RNA interference molecules and nucleic acid inhibitors useful in the methods as disclosed herein can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.)

In some circumstances, for example, where increased nuclease stability is desired, nucleic acids having nucleic acid analogs and/or modified internucleoside linkages can be preferred. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂-CH₂), 2′-O-alkyl, and 2′-deoxy-2′-fluoro′phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., 5,714,606 to Acevedo, et al, 5,378,825 to Cook, et al., 5,672,697 and 5,466,786 to Buhr, et al., 5, 777,092 to Cook, et al., 5,602,240 to De Mesmacker, et al., 5,610,289 to Cook, et al. and 5,858,988 to Wang, also describe nucleic acid analogs for enhanced nuclease stability and cellular uptake.

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a poIIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence, beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. One method of designing a siRNA molecule of the present invention involves identifying the 23 nucleotide sequence motif AA(N19)TT (where N can be any nucleotide), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but are not limited to the NCBI, BLAST, Derwent and GenSeq as well as commercially available oligosynthesis software such as OLIGOENGINE®, can also be used to select siRNA sequences against EST libraries to ensure that only one gene is targeted.

Delivery of RNA Interfering Agents:

In embodiments where the agent which increases the nuclear form of Creb312 is a RNAi agent, methods of delivering RNA interfering agents, e.g., an siRNA, or vectors containing an RNA interfering agent, to a target cell (e.g., cardiomyocytes and or a cell or cardiac cell or other desired target cells), can include, for example (i) injection of a composition containing the RNA interfering agent, e.g., an siRNA, or (ii) directly contacting the cell with a composition comprising an RNA interfering agent, e.g., an siRNA. In another embodiment, RNA interfering agents, e.g., an siRNA can be injected directly into any blood vessel, such as vein, artery, venule or arteriole, via, e.g., hydrodynamic injection or catheterization. In some embodiments siRNA agents can delivered to specific organs, for example the liver, bone marrow or systemic administration.

Administration can be by a single injection or by two or more injections. A RNA interfering (RNAi) agent can be delivered in a pharmaceutically acceptable carrier. One or more RNA interfering agents can be used simultaneously. A RNA interfering agent can also be delivered singly, or in combination with other RNA interfering agents, e.g., siRNAs, such as, for example siRNAs directed to other cellular genes.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501).

RNA interfering agents, for e.g., an siRNA, can also be introduced into cells via the vascular or extravascular circulation, the blood or lymph system, and the cerebrospinal fluid.

The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene. A skilled artisan would also know that RNAi molecules do not have to match perfectly to their target sequence. Preferably, however, the 5′ and middle part of the antisense (guide) strand of the siRNA is perfectly complementary to the target nucleic acid sequence.

Accordingly, the RNAi molecules functioning as an agent to increase nuclear Creb312 in the present invention are for example, but are not limited to, unmodified and modified double stranded (ds) RNA molecules including short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also can contain 3′ overhangs, preferably 3′UU or 3′TT overhangs. In one embodiment, the siRNA molecules of the present invention do not include RNA molecules that comprise ssRNA greater than about 30-40 bases, about 40-50 bases, about 50 bases or more. In one embodiment, the siRNA molecules of the present invention are double stranded for more than about 25%, more than about 50%, more than about 60%, more than about 70%, more than about 80%, more than about 90% of their length.

In another embodiment of the invention, an agent which increase the nuclear form of Creb312 can be a catalytic nucleic acid construct, such as, for example a ribozyme, which is capable of cleaving RNA transcripts and thereby preventing the production of wildtype protein. In some embodiments, a ribozyme can be targeted to and anneal with a particular sequence by virtue of two regions of sequence complementary to the target flanking the ribozyme catalytic site. After binding, the ribozyme cleaves the target in a site specific manner. The design and testing of ribozymes which specifically recognize and cleave sequences of the gene products described herein, for example for cleavage of the full length Creb312 gene to a variant that encodes the nuclear form of Creb312 (i.e. encodes SEQ ID NO: 3) or homologues or variants thereof can be achieved by techniques well known to those skilled in the art (for example Lleber and Strauss, (1995) Mol Cell Biol 15:540.551, the disclosure of which is incorporated herein by reference).

Assessment of Agents which Increase Nuclear Creb312 and/or Promote Cardiomyocyte Survival

In some embodiments, an agents which increases nuclear form of Creb312 useful in the methods and compositions of the present invention can be assessed using the BNP promoter assay as disclosed herein in the Examples. For example, an agent which increases the nuclear form of Creb312 can be identified by one of ordinary skill in the art as an agent which, in the presence of full length Creb312 (Creb312FL) increases transcription of a gene operatively linked to the BNP promoter (i.e. an agent which increases luciferase gene expression) as compared to a control agent or the absence of an agent. Alternatively, one of ordinary skill in the art can treat a cell expressing full length Creb312 and identify the presence of the nuclear form in the presence or absence of the agent, using western blot analysis according to the Examples and Example 3 herein, or another similar protein detection methods. In some embodiments, one can perform immunoblot and western blot analysis with an anti-Creb312 antibody, such as an anti-creb312 antibody which binds to and has affinity for both the full-length Creb312 polypeptide and the nuclear form of the Creb312 polypeptide, and an agent which increase the proportion of the nuclear form of Creb312 and decreases the full length Creb312 would be useful in the methods and compositions as disclosed herein. Alternatively, one can perform immunoblot and western blot analysis using an anti-Creb312 antibody which binds and has affinity for the nuclear form of the Creb312 polypeptide, and any agent which when added to a cell expressing full length Creb312 increases the level of nuclear form of Creb312 in the presence of the agent as compared to a control agent (or the absence of the agent) would identify that the agent would be useful in the methods and compositions as disclosed herein.

In some embodiments, an agent which increases the nuclear form of Creb312 can be assessed to identify if it increase the cardiomyocyte cultures, and/or increases proliferation of cardiomyocytes is useful in the present invention. By way of example, cardiomyocyte cultures can be prepared by one of ordinary skill in the art. For example, one such method is as follows:

Primary culture of neonatal rat ventricular myocytes (NRVCs) can be prepared by one of ordinary skill in the art. After preparation, cells are incubated in Dulbecco's modified Eagle's medium (DMEM) supplemented with 7% Fetal Calf Serum (FCS). Eighteen to 24 hours after preparation, cells were transfected with adenoviral vector at a multiplicity of infection (MOI) of 50 in DMEM (supplemented with 1% of FCS) for 16 hours and incubated in DMEM without serum for more than 12 hours.

In some embodiments, an agent useful in the methods as disclosed herein can be identified by assessing if the agent promotes survival of Creb312 null mutant (−/−) neonatal cardiomyocytes or Creb312 null mutant (−/−) neonatal rat ventricular myocytes (NRVCs) in response to physiological stress, such as mechanical stretch stress, or hormonal or hypoxic stress. Agents which promote or increase survival of Creb312 null mutant (−/−) neonatal cardiomyocytes or Creb312 null mutant (−/−) neonatal rat ventricular myocytes (NRVCs) to a comparable level of survival of wild type mice neonatal cardiomyocytes or wild type NRVCs are useful in the present invention. Alternatively, agents which increase the survival of Creb312 null mutant (−/−) neonatal cardiomyocytes or Creb312 null mutant (−/−) NRVCs as compared to a control agent or absence of the test agent are useful in the present invention. In some embodiments, the agents are contacted with Creb312 null mutant (−/−) NRVCs in an in vitro assay, and in alternative embodiments, an in vivo assay can be used to assess the agents, by administering the agents to a Creb312 null mutant (−/−) neonatal mouse in vivo and assessing the effect on the survival of cardiomyocytes as disclosed in Example 2.

Cell survival and survival of cardiomyocytes can be determined by any means known by a skilled artisan. One method for example, is TUNEL staining which identifies cells undergoing apoptotic cell death, thus a decrease in TUNEL positive cells identifies an agent which is able to promote cell survival and decrease apoptotic cell death as compared to the control agent or absence of agent. TUNEL staining for cultured cardiac myocytes cab be performed using the In Situ Cell Death detection kit (Roche) with some modifications. In brief, cells can be fixed by 4% paraformaldehyde in PBS, permeabilized with 0.1% Triton X-100 and incubated with anti-sarcomeric actinin antibody for 60 min followed by incubation with Cy3-conjugated anti-mouse IgG antibody. Cells can then incubated with TUNEL staining solution for 1 hr according to the manufacture's protocol. DAPI was used for nuclear staining. TUNEL staining for the frozen heart sections was performed as described previously 7 with some modifications. Cryo-sections (6 μm thickness) were fixed with 4% paraformaldehyde in PBS, permeabilized with 0.1% Triton X-100 and blocked with 6% skim milk. Anti-sarcomeric actin antibody was used for determination of myocytes followed by TUNEL and nuclear staining as described above. TUNEL positive myocytes were counted in randomly selected three fields of the slide and the experiments were repeated three times in duplicates.

Alternatively, one can assess if an agent is useful herein to promote cardiomyocyte proliferation and functions to increase the nuclear form of Creb312 in a mouse model of ischemia and reperfusion injury and evaluation of infarction size. An agent which decreases cardiac muscle injury and/or the size of infarct after ischemia is useful in the methods and compositions as disclosed herein. Such mice models of ischemia and reperfusion injury are well known by a skilled artisan, but by way of example only, one such a model is described as follows: Eight week old male C57BL/6 mice (obtained from Charles River Laboratories) can be intravenously injected through the jugular vein with a test agent which increases the nuclear form of Creb312. One such agent can be an adenovirus expressing the nuclear form of Creb312 (1.0×109 p.f.u./mouse). Serum nuclear Creb312 can be assayed by Western blot analysis five days after adenovirus delivery. Following detection of the test agent expression (i.e. expression of nuclear Creb312) the ischemia/reperfusion injury can be performed. Following anesthetization (pentobarbital 50 mg/kg i.p.) and intubation, the chest can be exteriorized and 8-0 monofilament suture ligated around the proximal left coronary artery (LCA) using a snare occluder. Ischemia followed by reperfusion can be accomplished by tightening the snare occluder for 30 min and then loosing it. Myocardial reperfusion can be confirmed by changes in ECG as well as by changes in appearance of the heart from pale to bright red. The suture can be left in place and chest was closed. For mouse myocardial infarction model, the LCA can be occluded by tight ligation without reperfusion. During the surgical procedure, the body temperature can be monitored and maintained at 37+/−1° C. Twenty four hours after reperfusion, the chest can be re-opened and the suture was re-tied. Evans Blue can be injected at the aortic root to determine the area at risk (AAR). The heart can then excised and incubated with 2,3,5-triphenyltetrazolium chloride (TTC) for 5 min at 37° C. to determine the infarction area (IA). Left ventricular area (LVA), AAR and IA can be determined by computerized planimetry using any image detection method analysis program known by a skilled artisan, for example Image J (Bethesda, Md., USA). An agent which decrease the size of at least one of; the infarction area (IA), LVA or AAE, or alternatively decreases myocardial cell death as compared to a control agent or in the absence of the agent would be useful in the methods and composition of the present invention.

Alternatively, one can assess if an agent is useful herein to promote cardiomyocyte proliferation and functions to increase the nuclear form of Creb312 in a mouse transverse aortic constriction model. An agent which decreases cardiac muscle injury and/or the size of infarct after ischemia is useful in the methods and compositions as disclosed herein. Such mouse transverse aortic constriction models are well known by a skilled artisan, but by way of example only, one such a model is described as follows: Following anesthetization (pentobarbital 50 mg/kg i.p.), the thoracic aorta can be reached by dissecting intercostal muscle. The identified aorta can then tied with a 7-0 silk suture along with a 26-gauge blunt needle which was subsequently removed to produce a pressure gradient at the site.

Use of Agents to Increase Nuclear Creb312 for the Treatment of Cardiac Injury and Other Ischemic Injury

In some embodiments, agents as disclosed herein can be used for the treatment or prevention of ischemic injury, such as cardiac ischemic or ischemia/reperfusion injury. In such embodiments, an agent which increases the nuclear form of Creb312 polypeptide or a functional fragment thereof as described herein can be administered to a subject in need thereof. Administration should commence as soon as possible following or during an ischemic event in order to have the best chance of benefit, but can also be beneficial at times thereafter. The administration can be by the same pathways as described above for metabolic therapies. However, intravenous administration or even localized cardiac administration can be advantageous. Due to the acute nature of the ischemic event, effective treatment of cardiac or other tissue for ischemic injury is not likely to require prolonged administration, although this is not ruled out for potential preventive benefit. Also, the dosages likely to be effective can be similar to those as determined in mouse models of cardiac injury or other ischemic injury and in some instances scaled up from in vivo usages in a mouse model, although it is also contemplated that much higher dosages can be initially administered to fully establish a cardioprotective effect before the damage becomes too extensive.

It is contemplated that anti-inflammatory or immunosuppressive agents can also be concurrently administered if necessary to counteract any adverse or systemic effects in cardiac patients receiving an agent that increases nuclear Creb312. Administration of a viral vector encoding an agent which increases the nuclear form of Creb312 in a mouse model is discussed herein. Viral vectors can also be employed in humans for cardioprotective therapy if so desired—an advantage is the fairly rapid production and secretion of large amounts of the recombinant protein in vivo.

Measurement of Cardiac Muscle Injury

In some embodiments, agents as disclosed herein that increase the level of the nuclear from of Creb312 can be used for the treatment or prevention of cardiac muscle injury by increasing cardiomyocyte proliferation and/or promoting survival of cardiomyocytes. Generally, although cardiac muscle biopsy would likely indicate the status of cardiac muscle after a treatment, such an invasive and potentially further-destructive approach in an individual with ischemic injury to the heart is clearly not warranted to monitor the effects of cardiac protective treatments as described herein. Instead, the efficacy of cardiac protection or therapy according to the methods described herein can be evaluated by following surrogate or indirect markers of cardiac health and function. For example, cardiac cell death, whether by necrosis or apoptosis, is generally accompanied by the release of cardiac enzymes, including cardiac creatine kinase (CK). Assays for cardiac enzymes are routinely used in the diagnosis of myocardial infarction and can be used to monitor the efficacy of cardiac protection according to the methods described herein. A decrease in cardiac enzymes (e.g., a 10% or greater decrease), or a lower level than is normally seen with an infarct of a given size, is indicative of effective treatment. Other markers include, for example, cardiac Troponin T (TnT), which is a marker of cardiac injury that is used as an alternative for CK. In addition, one can perform “echocardiographic analysis of ejection fraction” as a measure of cardiac injury, stabilization after injury or recovery after injury.

Of course, another marker of the efficacy of cardioprotective intervention as described herein is survival. Statistical survival rates for myocardial ischemic events are well established—when an individual or group of individuals treated according to the methods described herein survives beyond the expected time or at a greater than expected rate for an infarct of a given size and location, the treatment can be considered effective.

Vectors and Expression of Agents which Increase the Nuclear Form of Creb312 or Expression of Nuclear Form of Human Creb312

As discussed herein, in some embodiments, a nucleic acid encoding a Creb312(N) polypeptide is introduced into a cell and the Creb312(N) polypeptide expressed in order to promote survival of the cell or to increase the proliferation of the cell. Any means to introduce the nucleic acid into the cell can be used, for example by using gene therapy techniques such as use of an expression vector, or viral vector. In some embodiments, the nucleic acid can be introduced as naked DNA and by standard transfection methods.

Expression vectors compatible with eukaryotic cells, preferably those compatible with vertebrate cells, can be used to produce recombinant constructs for the expression of an agent which increases the nuclear form of Creb312 or alternatively recombinant constructs which express the nuclear form of Creb312 polypeptide or portions or derivatives thereof as described herein. Eukaryotic cell expression vectors are well known in the art and are available from several commercial sources. Typically, such vectors are provided containing convenient restriction sites for insertion of the desired DNA segment. These vectors can be viral vectors such as adenovirus, adeno-associated virus (AAV), pox virus such as an orthopox (vaccinia and attenuated vaccinia), avipox, lentivirus, murine moloney leukemia virus, etc. Alternatively, plasmid expression vectors can also be used.

Viral vector systems which can be utilized in the present invention include, but are not limited to, (a) adenovirus vectors; (b) retrovirus vectors; (c) adeno-associated virus vectors (AAV); (d) herpes simplex virus vectors (HSV); (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In one embodiment, the vector is an adenovirus or an adenoassiciated virus. Replication-defective viruses can also be advantageous.

The vector may or may not be incorporated into the cells genome. The constructs may include viral sequences for transfection, if desired. Alternatively, the construct may be incorporated into vectors capable of episomal replication, e.g. EPV and EBV vectors.

Constructs for the recombinant expression of an agent which increases the nuclear form of Creb312 or a construct for the recombinant expression of the nuclear form of Creb312 polypeptide will generally require regulatory elements, e.g., promoters, enhancers, etc., to ensure the expression of the construct in target cells. Other specifics for vectors and constructs are described in further detail below.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. In some embodiments, it can be advantageous to direct expression of a Creb312(N) polypeptide in a tissue- or cell-specific manner. Cardiac-specific expression can be achieved, for example, using the cardiac NCX1 promoter (Nicholas et al., 1998, Am. J. Physiol. Heart Circ. Physiol. 274: H217-H232), the cardiac myosin light chain 2 promoter (Griscelli et al., 1998, Hum. Gene Ther. 9: 1919-1928) or other cardiac-specific promoter known in the art.

In a specific embodiment, viral vectors that contain nucleic acid sequences encoding an agent which increases the nuclear form of Creb312 or a viral vector which expresses the nuclear form of Creb312 polypeptide or portions or derivatives thereof are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding an agent which increases the nuclear form of Creb312 or the nucleic acid sequences encoding the human nuclear form of Creb312 polypeptide or portions or derivatives thereof are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

The production of a recombinant retroviral vector carrying a gene of interest is typically achieved in two stages. First, sequence encoding an agent which increases the nuclear form of Creb312 or a sequence encoding the nuclear form of human Creb312 polypeptide or portions or derivatives thereof can be inserted into a retroviral vector which contains the sequences necessary for the efficient expression of the metabolic regulators (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (−PBS), a 3′ [prime] regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant retroviral vector, the vector DNA is introduced into a packaging cell line. Packaging cell lines provide viral proteins required in trans for the packaging of viral genomic RNA into viral particles having the desired host range (e.g., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permits entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles which contain viral genomic RNA derived from one virus encapsidated by the envelope proteins of another virus are said to be pseudotyped virus particles.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia virus, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In another embodiment, lentiviral vectors are used, such as the HIV based vectors described in U.S. Pat. Nos. 6,143,520; 5,665,557; and 5,981,276, which are herein incorporated by reference. Use of Adeno-associated virus (AAV) vectors is also contemplated (Walsh et al., Proc. Soc. Exp. Biol. Med. 204:289-300 (1993); U.S. Pat. No. 5,436,146).

Another approach to gene therapy involves transferring a gene or nucleic acid sequence to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells can then be transplanted or delivered to a subject patient.

U.S. Pat. No. 5,676,954 (which is herein incorporated by reference) reports on the injection of genetic material such as naked DNA, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are all herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein all incorporated by reference) provide methods for delivering DNA-cationic lipid complexes to mammals. Such cationic lipid complexes or nanoparticles can also be used to deliver protein.

A gene or nucleic acid sequence can be introduced into a target cell by any suitable method. For example, a construct expressing an agent that increases the nuclear form of Creb312 or a construct expressing the nuclear form of human Creb312 or a functional fragment or derivative thereof can be introduced into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A nucleic acid encoding an Creb312(N) polypeptide can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

In certain embodiments, a gene or nucleic acid sequence encoding an agent which increases the nuclear form of Creb312 or a nucleic acid sequence encoding the nuclear form of human Creb312 polypeptide or portions or derivatives thereof can also be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

Any method known in the art for the therapeutic delivery of agents such as proteins and/or nucleic acids can be used for the delivery of a polypeptide or nucleic acid encoding an agent which increases the nuclear form of Creb312 or a nucleic acid sequence encoding the nuclear form of human Creb312 polypeptide or portions or derivatives thereof for cardioprotection in a subject can be used, and include without limitation, cellular transfection, gene therapy, direct administration with a delivery vehicle or pharmaceutically acceptable carrier, indirect delivery by providing recombinant cells comprising a nucleic acid encoding a targeting fusion polypeptide of the invention.

Various delivery systems are known and can be used to directly administer therapeutic polypeptides, e.g., encapsulation in liposomes, microparticles, microcapsules, recombinant cells capable of expressing the compound, and receptor-mediated endocytosis (see, e.g., Wu and Wu, 1987, J. Biol. Chem. 262:4429-4432). Methods of introduction can be enteral or parenteral and include but are not limited to intradermal, intramuscular, intraperitoneal, intravenous, subcutaneous, pulmonary, intranasal, intraocular, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes. In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105).

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked nucleic acid encoding an agent which increases the nuclear form of Creb312 or a nucleic acid sequence encoding the nuclear form of human Creb312 polypeptide or portions or derivatives thereof into the selected expression/delivery vector, many equivalent vectors for the practice of the methods described herein can be generated.

It will be appreciated by those of skill that cloned genes readily can be manipulated to alter the amino acid sequence of a protein. The nucleic acid sequence for the nuclear form of Creb312 can be readily manipulated by a variety of well known techniques for in vitro mutagenesis, among others, to produce variants of the naturally occurring human protein, herein referred to as muteins or variants or mutants of the nuclear form of Creb312, which may be used in accordance with the methods and compositions described herein. Similarly, one can also manipulate the nucleic acid sequence or a gene which when expressed encodes an agent which increases the nuclear form of Creb312. Such nucleic acid sequences which encode an agent which increases the nuclear form of Creb312 and thus be readily manipulated by a variety of well known techniques for in vitro mutagenesis, among others, to produce variants of the naturally occurring protein.

The variation in amino acid residues of an agent which increases the nuclear form of Creb312 or a variation in the amino acid sequence or primary structure of muteins of the nuclear form of Creb312 useful in the invention, for instance, may include deletions, additions and substitutions. The substitutions may be conservative or non-conservative. The differences between the natural protein and the mutein generally conserve desired properties, mitigate or eliminate undesired properties and add desired or new properties.

In some embodiments, where the agent which increases the nuclear form of Creb312 is a nuclear from of Creb312 polypeptide corresponding to SEQ ID NO: 3 or a variant or mutant thereof, the agent or nuclear form of Creb312 polypeptide can also be a fusion polypeptide, fused, for example, to a polypeptide that targets the product to a desired location, or, for example, a tag that facilitates its purification, if so desired. Fusion to a polypeptide sequence that increases the stability of the agent, or nuclear form of Creb312 polypeptide is also contemplated. For example, fusion to a serum protein, e.g., serum albumin, can increase the circulating half-life of the agent, or the nuclear form of Creb312 polypeptide. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use.

In some embodiments, the agent which increases the nuclear form of Creb312 or the nuclear form of Creb312 polypeptide or a functional fragment thereof can be a fusion protein which increases its affinity to form oligomers, such as dimers, trimer and oliogomers of more than three proteins. In a particular embodiment, an agent can be a nuclear form of Creb312 or a functional fragment thereof fused to a Fc protein (as disclosed in U.S. Pat. Nos. 4,625,014, 5,057,301 and 5,514,363, which are incorporated herein by reference) to increase the formation of homodimers of the nuclear form of Creb312 as discussed are formed in vitro in the Examples. In alternative embodiments, an agent can be a nuclear form of Creb312 or a functional fragment thereof fused to any protein which increases the formation of homodimers or oligomers comprised of the nuclear form of Creb312. For example, the nuclear form of Creb312 can be fused to a second fusion partner, such as a carrier molecule to enhance its bioavailability. Such carriers are known in the art and include poly (alkyl) glycol such as poly ethylene glycol (PEG). Fusion to serum albumin can also increase the serum half-life of therapeutic polypeptides.

In some embodiments, the nuclear form of Creb312 polypeptide can also be fused to a second fusion partner, for example, to a polypeptide that targets the product to a desired location, or, for example, a tag that facilitates its purification, if so desired. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use.

As used herein, the term “conjugate” or “conjugation” refers to the attachment of two or more entities to form one entity. For example, the methods of the present invention provide conjugation of the nuclear form of Creb312 polypeptide (i.e. SEQ ID NO: 3 or fragments, derivatives or variants thereof) joined with another entity, for example a moiety such as a first fusion partner that makes the nuclear form of Creb312 polypeptide stable, such as Ig carrier particle, for example IgG1 Fc. The attachment can be by means of linkers, chemical modification, peptide linkers, chemical linkers, covalent or non-covalent bonds, or protein fusion or by any means known to one skilled in the art. The joining can be permanent or reversible. In some embodiments, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention.

According to the present invention, the nuclear form of Creb312 polypeptide (i.e. SEQ ID NO: 3 or fragments, derivatives or variants thereof), can be linked to the first fusion partner via any suitable means, as known in the art, see for example U.S. Pat. Nos. 4,625,014, 5,057,301 and 5, 514,363, which are incorporated herein in their entirety by reference. For example, the nuclear form of Creb312 polypeptide can be covalently conjugated to the IgG1 Fc, either directly or through one or more linkers. In one embodiment, a nuclear form of Creb312 polypeptide as disclosed herein is conjugated directly to the first fusion partner (e.g. Fc), and in an alternative embodiment, a nuclear form of Creb312 polypeptide as disclosed herein can be conjugated to a first fusion partner (such as IgG1 Fc) via a linker, e.g. a transport enhancing linker. A large variety of methods for conjugation of a nuclear form of Creb312 polypeptide as disclosed herein with a first fusion partner (e.g. Fc) are known in the art. Such methods are e.g. described by Hermanson (1996, Bioconjugate Techniques, Academic Press), in U.S. Pat. No. 6,180,084 and U.S. Pat. No. 6,264,914 which are incorporated herein in their entirety by reference and include e.g. methods used to link haptens to carriers proteins as routinely used in applied immunology (see Harlow and Lane, 1988, “Antibodies: A laboratory manual”, Cold Spring Harbor Laboratory Press, Cold Spring Harbor, N.Y.). It is recognized that, in some cases, a nuclear form of Creb312 polypeptide can lose efficacy or functionality upon conjugation depending, e.g., on the conjugation procedure or the chemical group utilized therein. However, given the large variety of methods for conjugation the skilled person is able to find a conjugation method that does not or least affects the efficacy or functionality of the entities, such as the nuclear form of Creb312 polypeptide to be conjugated.

Suitable methods for conjugation of a nuclear form of Creb312 polypeptide as disclosed herein with a first fusion partner (e.g. Fc) include e.g. carbodimide conjugation (Bauminger and Wilchek, 1980, Meth. Enzymol. 70: 151-159). Alternatively, a moiety can be coupled to a targeting agent as described by Nagy et al., Proc. Natl. Acad. Sci. USA 93:7269-7273 (1996), and Nagy et al., Proc. Natl. Acad. Sci. USA 95:1794-1799 (1998), each of which are incorporated herein by reference. Another method for conjugating one can use is, for example sodium periodate oxidation followed by reductive alkylation of appropriate reactants and glutaraldehyde crosslinking.

One can use a variety of different linkers to conjugate a nuclear form of Creb312 polypeptide as disclosed herein with a first fusion partner (e.g. Fc), for example but not limited to aminocaproic horse radish peroxidase (HRP) or a heterobiofunctional cross-linker, e.g. carbonyl reactive and sulfhydryl-reactive cross-linker. Heterobiofunctional cross linking reagents usually contain two reactive groups that can be coupled to two different function targets on proteins and other macromolecules in a two or three-step process, which can limit the degree of polymerization often associated with using homobiofunctional cross-linkers. Such multi-step protocols can offer a great control of conjugate size and the molar ratio of components.

The term “linker” refers to any means to join two or more entities, for example a nuclear form of Creb312 polypeptide as disclosed herein with a first fusion partner (e.g. Fc). A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling. It will be appreciated that modification which do not significantly decrease the function of the nuclear form of Creb312 polypeptide as disclosed herein or the first fusion partner (e.g. Fc) are preferred.

In another aspect, biodegradable or absorbable polymers can provide extended, often localized, release of polypeptide agents. The potential benefits of an increased half-life or extended release for a therapeutic agent are clear. A potential benefit of localized release is the ability to achieve much higher localized dosages or concentrations, for greater lengths of time, relative to broader systemic administration, with the potential to also avoid possible undesirable side effects that may occur with systemic administration.

Bioabsorbable polymeric matrix suitable for delivery of agents which are polypeptides, or the nuclear form of Creb312 polypeptide corresponding to SEQ ID NO: 3 or variants or muteins thereof can be selected from a variety of synthetic bioabsorbable polymers, which are described extensively in the literature. Such synthetic bioabsorbable, biocompatible polymers, which may release proteins over several weeks or months can include, for example, poly-α-hydroxy acids (e.g. polylactides, polyglycolides and their copolymers), polyanhydrides, polyorthoesters, segmented block copolymers of polyethylene glycol and polybutylene terephtalate (Polyactive™), tyrosine derivative polymers or poly(ester-amides). Suitable bioabsorbable polymers to be used in manufacturing of drug delivery materials and implants are discussed e.g. in U.S. Pat. Nos. 4,968,317, 5,618,563, among others, and in “Biomedical Polymers” edited by S. W. Shalaby, Carl Hanser Verlag, Munich, Vienna, New York, 1994 and in many references cited in the above publications. The particular bioabsorbable polymer that should be selected will depend upon the particular patient that is being treated.

### Uses of Creb312 to Promote Survival of Cells to Mechanical Stress or Promote Proliferation of Cardiomyocytes

In some embodiments it would be desirable to repopulate a population of diseased or injured cardiac cells, such as cardiomyocytes in a diseased or injured heart with a stem cell population which is capable of regenerating into cardiomyocytes and blood vessels, reverse ventricular remodeling, or reduce apoptosis of existing cells. Both myogenesis and angiogenesis may be required to restore cardiac function in patients with transmural scar tissue. Thus, cells that are capable of inducing angiogenesis and forming muscle-like cells and endothelial cells may be useful to reverse cardiac dysfunction in patients with CHF or myocardial infarction (MI). Examples of such cells include, for example, cells which have the capacity to differentiate along more than one heart lineage. The generation of diverse endothelial, smooth muscle, and cardiac cell lineages in discrete heart chambers and vessels is a critical step in cardiogenesis. Examples of such cells include, for example, 1s11+ cardiomyocyte cells which can differentiation into three different heart cell types as disclosed in International Application WO 2008/054819 and Provisional Application, 61/036,668 filed on Mar. 14, 2008. However, in most instances, the survival and long-term viability of transplanted cells may be limited as these cells when transplanted into the heart are exposed to continuous mechanical stress during heart contraction.

Accordingly, one aspect of the present invention is the use of a Creb312(N) polypeptide in a transplanted cell, such as stem cell, such as a cardiac progenitor cell to promote the survival of the cell after transplantation into a subject as a cell-based therapeutic intervention for cardiac diseases and disorders. Expressing a Creb312(N) polypeptide in a cell to be transplanted into a subject would increase its effectiveness post-transplantation by decreasing its chance of cell death due to mechanical stress (i.e. promoting its survival) post-transplantation into a subject. As cell-based therapies are being developed for the treatment of diseased or lost cardiomyocytes (by restoring or enhancing cardiac muscle function or cell mass, for example by regenerating existing cardiomyocytes, or replacing lost cardiomyocytes) using cell-based therapy, use of transplanted cells which express a Creb312(N) polypeptide which can maintain viability when exposed to mechanical stress that occurs due to heart contraction is highly beneficial.

Additionally, another aspect of the present invention relates to methods to use Creb312(N) to promote survival of a transplanted cell to mechanical stress, such as stretch mechanical stress, and/or to promote the proliferation of cardiomyocytes such as postnatal cardiomyocytes. In some embodiments, an agent which increase Creb312(N) polypeptide in the cell to be transplanted can be used in the preparation of cardiac progenitors, such as Isl1+ progenitors which are subsequently used in cell replacement therapy. For example in one embodiment of this aspect of the present invention, a cardiogenic progenitor useful in cell replacement therapy, is a Isl1⁺ cardiomyocyte progenitor cells or an atrial myocyte-derived Isl1⁺/SNL⁺ atrial progenitor which can differentiate into three different heart cell types as disclosed in International Application WO 2008/054819 and Provisional Application, 61/036,668 which are incorporated herein in their entirety by reference, can be administered Creb312(N) polypeptide or be genetically modified to comprise a nucleic acid encoding Creb312(N) operatively linked to a regulatory sequence (such as a tissue specific promoter or inducible promoter) as disclosed herein, thereby increasing the survival of these cells to mechanical stretch stress, such as the exposure to mechanical stress following transplantation into a subject.

Thus, in some embodiments, a nucleic acid encoding a Creb312(N) polypeptide is introduced into a cardiac progenitor, such as a human Isl1+ primordial cardiac progenitor or their progeny, as disclosed in U.S. Provisional applications 61/185,752 or 61/256,960 which are incorporated herein in their entirety by reference. In some embodiments, a nucleic acid encoding a Creb312(N) polypeptide is introduced into a cardiac progenitor, such as a Isl1+ cardiac progenitor or their progeny as disclosed in Intetnational Applications WO/2008/054819 and WO/2009/114673, which are incorporated herein in their entirety by reference.

Accordingly, in one embodiment of this aspect and all aspects described herein an agent which increases a Creb312(N) polypeptide can be used in combination with cardiomyocyte precursor cells, such as Isl1+ primordial cardiac progenitor cells, or Isl1⁺/Nkx2.5⁺/Flk⁺ or Isl1⁺/SNL⁺ atrial progenitors for the production of a pharmaceutical composition which can be used for transplantation into subjects in need of cardiac tissue transplantation, for example but not limited to subjects with congenital and acquired heart disease and subjects with vascular diseases. In some embodiments, the cardiomyocyte precursor cells, such as Isl1⁺/Nkx2.5⁺/Flk or Isl1⁺/SNL⁺ atrial progenitors can be genetically modified, for example genetically modified Isl1⁺/Nkx2.5⁺/Flk or Isl1⁺/SNL⁺ atrial progenitors which express either an agent which increases Creb312(N) in the cell, or alternatively express the Creb312(N) polypeptide (i.e. SEQ ID NO: 3) operatively linked to a suitable promoter, such as an inducible promoter or a cardiac specific promoter. In another aspect, the subject may have or be at risk of heart disease and/or vascular disease, and the subject is a mammal, and in other embodiments the mammal is a human.

The use of cardiomyocyte progenitor cells treated with an agent which increases Creb312(N), or cardiomyocyte progenitor cells expressing the Creb312(N) polypeptide (i.e. SEQ ID NO: 3) as disclosed herein provides advantages over existing methods because such cells have an increased chance of survival in response to the mechanical stress placed upon them due to the repeated heart contraction which they are exposed to following transplantation into the heart or a similar location during cell replacement therapy for a cardiovascular disease or disorder. This is highly advantageous as it provides a increased survival of a transplanted progenitor and/or cardiac muscle cell or population of transplanted progenitors once implanted, preventing a decrease in viability of cells post transplantation which normally occurs. Accordingly, administering an agent which increases a Creb312(N) polypeptide or administering a Creb312(N) polypeptide to a cardiovascular progenitor or an ES cell, or alternatively genetically modifying such cells to express a Creb312(N) polypeptide or a biologically active fragment or variant thereof under a suitable promoter (i.e. an inducible promoter or tissue-specific promoter) is useful to increase the survival of cells post-transplantation into the heart.

Accordingly, in some aspects and all other aspects described herein, the methods and composition are provided enable ex vivo genetic manipulation of cardiac progenitor cells and/or ES cells to express nuclear Creb312(N) polypeptide before transplantation into the heart. In such an embodiment, a cell which is administered an agent which increases a Creb312(N) polypeptide in the cell, or a cell which has a nucleic acid encoding a Creb312(N) polypeptide introduced into the cell prior to transplantation (i.e. transplantation into the heart) will likely have an increased survival in response to mechanical stress which occurs during the normal muscle heart contraction. Thus, the methods and composition as disclosed herein are useful in cell-based therapy for regeneration of heart structures without the reduced viability of the implanted cells post transplantation due to a reduction in mechanical stretch stress in the newly transplanted cells in a beating heart.

In another embodiment of this aspect and all aspects described herein, the methods and composition to increase a Creb312(N) polypeptide in a cell are useful in treating any cell before transplantation in a cell-based therapy for regeneration of any type of muscle (such as smooth muscle, cardiac muscle, striated muscle, including atrial, ventricular, outflow tract and conduction systems) or any tissue where the implanted cells will be subjected to mechanical stretch stress, such as any skin cell bone cell, as well as cells for tendon, cartilage, ligament and other tissue cell-based regeneration procedures.

In another embodiment of this aspect and all aspects described herein, the present invention relates to a method of treating a cardiovascular disease or disorder (such as a circulatory disorder) comprising administering an effective amount of a composition comprising an agent which increases Creb312(N) to a subject with a circulatory disorder. In a further embodiment, the invention provides a method for treating myocardial infarction, comprising administering a composition comprising an agent which increases Creb312(N) to a subject having a myocardial infarction in an effective amount sufficient to increase the proliferation of cardiomyocytes in the heart of the subject. In a further embodiment, the invention provides a method for treating myocardial infarction, comprising administering a composition comprising at least one cell, such as a cardiac progenitor which have been modified ex vivo to express a Creb312(N) polypeptide to a subject having a myocardial infarction in an effective amount sufficient to increase the proliferation of cardiomyocytes in the heart of the subject.

In some embodiments, the methods as disclosed herein further provides a method of treating an injured tissue in a subject comprising: (a) determining a site of tissue injury in the subject; and (b) administering an agent which increases Creb312(N) as disclosed herein in a composition into and around the site of tissue injury, wherein the an agent which increases Creb312(N) promotes the proliferation of cardiomyocytes. In some embodiments, the methods as disclosed herein further provides a method of treating an injured tissue in a subject comprising: (a) determining a site of tissue injury in the subject; and (b) administering a cardiac progenitor cell as disclosed herein in a composition into and around the site of tissue injury, wherein the cardiac progenitor has been modified ex vivo by introduction of a nucleic acid to encode Creb312 and to express a Creb312(N) polypeptide. In one embodiment, the site of tissue injury is injury to cardiac muscle. In a further embodiment, the tissue injury is a myocardial infarction, cardiomyopathy or congenital heart disease

In one embodiment of the above methods, the subject is a human. In alternative embodiments, an agent which increases Creb312(N) can be use to treat circulatory disorder is selected from the group consisting of cardiomyopathy, myocardial infarction, and congenital heart disease. In some embodiments, the circulatory disorder is a myocardial infarction. The methods as disclosed herein provides that an agent which increases Creb312(N) promotes proliferation of cardiomyocytes, or promotes survival of cells to mechanical stress for the treatment of myocardial infarction by reducing the size of the myocardial infarct. It is also contemplated that an agent which increases Creb312(N) promotes proliferation of cardiomyocytes, or promotes survival of cells to mechanical stress can be used to treat myocardial infarction by reducing the size of the scar resulting from the myocardial infarct. The methods as disclosed herein also encompasses that an agent which increases Creb312(N) is administered directly to heart tissue of a subject, or is administered systemically, or administered ventricular wall of the heart. In some embodiments, the agents are administered as part of a composition with cells to be transplanted into subject. In alternative embodiments, an agent which increases Creb312(N) is administered directly to heart tissue of a subject which has had a transplantation of cell to the heart.

In some embodiments, the methods as disclosed herein can be used to treat circulatory damage in the heart or peripheral vasculature which occurs as a consequence of genetic defect, physical injury, environmental insult or damage from a stroke, heart attack or cardiovascular disease (most often due to ischemia) in a subject, the method comprising administering an agent which increases Creb312(N) in combination with administering (i.e. transplanting), an effective number or amount of cardiovascular progenitors and/or their progeny (such as smooth muscle cells or cardiomyocytes) to a subject. Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

In some embodiments, the effects of an agent which increases Creb312(N) or the effect of introduction of a nucleic acid encoding Creb312(N) polypeptide which is administered directly cells, e.g. heart tissue cells or cardiac progenitor cells of a subject, can be demonstrated by, but not limited to, one of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of an agent which increases Creb312(N) in combination with cellular therapy of a cardiovascular progenitor cell can be evident over the course of days to weeks to months or years after the procedure. However, beneficial effects may be observed as early as several hours after the procedure, and may persist for several years.

In some embodiments, an agent which increases Creb312(N) can be used in combination with cells to be transplanted into the heart for tissue reconstitution or regeneration in a human subject or other subject in need of such treatment. In some embodiments, an agent which increases Creb312(N) can be used in combination with cells to be transplanted into the heart are administered in a manner that permits the transplanted cells to graft or migrate to the intended tissue site and reconstitute or regenerate the functionally deficient area. Special devices are available that are adapted for administering cells capable of reconstituting cardiac function directly to the chambers of the heart, the pericardium, or the interior of the cardiac muscle at the desired location. In some embodiments, an agent which increases Creb312(N) can be used in combination with cells to be transplanted into the heart can be administered to a recipient heart by intracoronary injection, e.g. into the coronary circulation. In some embodiments, an agent which increases Creb312(N) can be used in combination with cells to be transplanted into the heart can also be administered by intramuscular injection into the wall of the heart.

In some embodiments, cells to be transplanted into the heart are cardiac cells or cardiac progenitor cells which have been modified to express a Creb312(N) polypeptide or a biologically functional fragment or variant thereof. For example, cardiac cells or cardiac progenitor cells which have been modified to express a Creb312(N) polypeptide can be modified to specifically or restrict expression of a Creb312(N) polypeptide in heart tissues or in instances when the cell is exposed to stress, such as mechanical or stretch-induced stress.

In some embodiments, a cell which is to be transplanted into a subject comprises a nucleic acid encoding a Creb312(N) polypeptide or a biologically functional fragment or variant thereof, which is operatively linked to a regulatory sequence such as a promoter. In some embodiments, a promoter is a cardiac tissue-specific promoter, such that the Creb312(N) polypeptide is expressed in cardiac tissue. In alternative embodiments, a promoter is a constitutive promoter, or a inducible promoter or a heterologous promoter as defined herein. In alternative embodiments, a promoter is a stress-induced promoter, so that the Creb312(N) polypeptide is expressed in stressful conditions, such as exposure to mechanical stress and/or stretch induced stress. In some embodiments, a promoter is a drug-induced promoter commonly known to one of ordinary skill in the art such as a tetracycline promoter and the like.

The compositions as disclosed herein can have a variety of uses in clinical therapy, research, development, and commercial purposes. For therapeutic purposes, for example, an agent which increases Creb312(N) can be used to enhance tissue maintenance or repair of cardiac muscle for any perceived need, such as an inborn error in metabolic function, the effect of a disease condition, or the result of significant trauma. An agent which increases Creb312(N) can be administered to the subject not only help restore function to damaged or otherwise unhealthy tissues, but also facilitate remodeling of the damaged tissues and promote survival of newly formed (i.e. newly propagated) cells or newly transplanted cells.

To determine the suitability of an agent which increases Creb312(N) for therapeutic administration, the agent which increases Creb312(N) can first be tested in a suitable cell-transplantation animal model. At one level, transplanted cells in the presence of an agent which increases Creb312(N) are assessed for their ability to survive and maintain their phenotype in vivo. In some embodiments, the cell compositions in the presence of an agent which increases Creb312(N) can be administered to immunodeficient animals (such as nude mice, or animals rendered immunodeficient chemically or by irradiation). Tissues are harvested after a period of regrowth, and assessed as to whether the administered cells or progeny thereof are still present. This can be performed by transplanting cells which express a detectable label (such as green fluorescent protein, or beta-galactosidase); that have been prelabeled (for example, with BrdU or [3H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

In embodiments, the suitability of an agent which increases Creb312(N) to promote survival of cells post-transplantation can also be determined in an animal model by assessing the degree of cardiac recuperation that ensues from treatment with the cells of the invention. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N2 on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

In some embodiments, an agent which increases Creb312(N) as disclosed herein may be administered in any physiologically acceptable excipients. The cells may be introduced by injection, catheter, or the like.

An agent which increases Creb312(N) as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition as disclosed herein can also comprise or be accompanied with one or more other ingredients that facilitate the engraftment or functional mobilization of the cells. Suitable ingredients include matrix proteins that support or promote adhesion of the cells, or complementary cell types, especially endothelial cells. In another embodiment, the composition may comprise resorbable or biodegradable matrix scaffolds.

As discussed above, in some embodiments, any cell used for transplantation, such as ES-cells or cardiovascular progenitors, can be genetically altered in order to introduce genes which encode an agent which increases Creb312(N), such as the expression of the Creb312(N) polypeptide itself to promote survival in response to mechanical stress. Accordingly, such cells are genetically modified pre-transplantation to enhance survival in response to mechanical stress. Cells can be genetically altered by any means known by one of ordinary skill in the art, for example by transfection or transduction with a suitable vector, homologous recombination, or other appropriate technique, so that they express a gene of interest such as an agent which increases Creb312(N), or the expression of the Creb312(N) polypeptide. Gene therapy can be used to either modify a cell to replace a gene product, to facilitate regeneration of tissue, to treat disease, or to improve survival of the cells following implantation into a subject (i.e. prevent rejection). By way of an example only, a vector is designed using the known encoding sequence for the desired gene encoding an agent which increases Creb312(N) (i.e. the expression of the Creb312(N) polypeptide) operatively linked to a promoter that is either pan-specific or specifically active in the cell to be transplanted. Many vectors useful for transferring exogenous genes into target mammalian cells are available. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PAl2 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bcl-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

Another aspect of the present invention relates to the administration of an agent which increases Creb312(N), or Creb312(N) polypeptide as disclosed herein either systemically or to a target anatomical site. An agent which increases Creb312(N), or Creb312(N) polypeptide can be administered to a subject along with cells to be transplanted for grafting into or nearby a subject's heart, for example, or may be administered systemically, such as, but not limited to, intra-arterial or intravenous administration. In alternative embodiments, an agent which increases Creb312(N), or Creb312(N) polypeptide as disclosed herein can be administered in various ways as would be appropriate to implant in the cardiovascular system, including but not limited to parenteral, including intravenous and intraarterial administration, intrathecal administration, intraventricular administration, intraparenchymal, intracranial, intracisternal, intrastriatal, and intranigral administration. Optionally, the cardiovascular stem cells are administered in conjunction with an immunosuppressive agent.

An agent which increases Creb312(N), or Creb312(N) polypeptide as disclosed herein can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is defined in the definitions sections and is determined by such considerations as are known in the art. The amount must be effective to halt the disease progression and/or to achieve improvement, including but not limited to improved survival rate or more rapid recovery, or improvement or elimination of symptoms and other indicators as are selected as appropriate measures by those skilled in the art. Administration to a subject of an agent which increases Creb312(N), or Creb312(N) polypeptide as disclosed herein can take place but is not limited to the following locations: clinic, clinical office, emergency department, hospital ward, intensive care unit, operating room, catheterization suites, and radiologic suites.

### Formulations of Compositions and Agents

The present invention provides therapeutic compositions useful for practicing the therapeutic methods described herein. Therapeutic compositions of the present invention contain a physiologically tolerable carrier together with an agent which increases the nuclear form of Creb312 polypeptide or a vector capable of expressing an agent which increases the nuclear form of Creb312, such as a vector expressing the nuclear form of Creb312 as described herein, dissolved or dispersed therein as an active ingredient. In a preferred embodiment, the therapeutic composition is not immunogenic when administered to a mammal or human patient for therapeutic purposes.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution or suspension in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. In addition, if desired, the composition can contain minor amounts of auxiliary substances such as wetting or emulsifying agents, pH buffering agents and the like which enhance the effectiveness of the active ingredient.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. For topical application, the carrier may be in the form of, for example, and not by way of limitation, an ointment, cream, gel, paste, foam, aerosol, suppository, pad or gelled stick.

The amount of an agent which increases the nuclear form of Creb312 that will be effective in the treatment of a particular disorder or condition will depend on the nature of the disorder or condition, and can be determined by standard clinical techniques. In addition, assays such as those discussed herein may optionally be employed to help identify optimal dosage ranges.

The precise dose to be employed in the formulation will also depend on the route of administration, and the seriousness of the disease or disorder, and should be decided according to the judgment of the practitioner and each subject's circumstances. Suitable dosage ranges for administration of agents are generally about 0.01 μg/kg body weight to 1.0 mg/kg body weight. In some embodiments, the suitable range for administration is 5 μg/kg body weight to 30 μg/kg body weight. Effective doses may be extrapolated from dose-response curves derived from in vitro or animal model test bioassays or systems.

The route of administration can be any route known to persons skilled in the art, for example but not limited to parenteral, including intravenous and intraarterial administration, intrathecal administration, intraventricular administration, intraparenchymal, intracranial, intracisternal, intrastriatal, and intranigral administration.

Agents, for example agents increasing the nuclear form of Creb312 as disclosed herein, can be used as a medicament or used to formulate a pharmaceutical composition with one or more of the utilities disclosed herein. They can be administered in vitro to cells in culture, in vivo to cells in the body, or ex vivo to cells outside of an individual that can later be returned to the body of the same individual or another. Such cells can be disaggregated or provided as solid tissue.

In some embodiments, agents increasing the nuclear form of Creb312 as disclosed herein can be used to produce a medicament or other pharmaceutical compositions. Use of agents increasing the nuclear form of Creb312 which further comprise a pharmaceutically acceptable carrier and compositions which further comprise components useful for delivering the composition to an individual are known in the art. Addition of such carriers and other components to the agents as disclosed herein is well within the level of skill in this art.

Pharmaceutical compositions can be optionally administered as a formulation adapted for passage through the blood-brain barrier or direct contact with the endothelium. In some embodiments, the compositions may be administered as a formulation adapted for systemic delivery. In some embodiments, the compositions may be administered as a formulation adapted for delivery to specific organs, for example but not limited to the heart, or intracardiac delivery.

Alternatively, pharmaceutical compositions can be added to the culture medium of cells ex vivo. In addition to the active agent, such compositions can contain pharmaceutically-acceptable carriers and other ingredients known to facilitate administration and/or enhance uptake (e.g., saline, dimethyl sulfoxide, lipid, polymer, affinity-based cell specific-targeting systems). The composition can be incorporated in a gel, sponge, or other permeable matrix (e.g., formed as pellets or a disk) and placed in proximity to the target cells, such as cardiomyocytes for sustained, local release. In some embodiments, a composition comprising agents which increase the nuclear form of Creb312 can be administered in a single dose or in multiple doses which are administered at different times.

Pharmaceutical compositions comprising agents which increase the nuclear form of Creb312 can be administered by any known route. By way of example, a composition can be administered by a mucosal, pulmonary, topical, or other localized or systemic route (e.g., enteral and parenteral). The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of the agents as disclosed herein such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

Suitable choices in amounts and timing of doses, formulation, and routes of administration can be made with the goals of achieving a favorable response in the subject with a cardiovascular disease, for example a subject with myocardial infarction or CHF or a risk thereof (i.e., efficacy), and avoiding undue toxicity or other harm thereto (i.e., safety). Therefore, “effective” refers to such choices that involve routine manipulation of conditions to achieve a desired effect.

A bolus of the a composition or formulation comprising agents which increase the nuclear form of Creb312 administered to an individual over a short time once a day is a convenient dosing schedule. Alternatively, the effective daily dose can be divided into multiple doses for purposes of administration, for example, two to twelve doses per day. Dosage levels of active ingredients in a pharmaceutical composition can also be varied so as to achieve a transient or sustained concentration of the compound or derivative thereof in an individual, especially in and around vascular endothelium of the brain, and to result in the desired therapeutic response or protection. But it is also within the skill of the art to start doses at levels lower than required to achieve the desired therapeutic effect and to gradually increase the dosage until the desired effect is achieved.

The amount of an agent which increases the nuclear form of Creb312 to be administered to cells or a subject is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration, and the like. It will also be understood that the specific dose level to be achieved for any particular individual subject can depend on a variety of factors, including age, gender, health, medical history, weight, combination with one or more other drugs, and severity of disease.

As discussed above, efficacy of treatment can be measured as an improvement in morbidity or mortality (e.g., lengthening of survival curve for a selected population with cardiovascular diseases or disorders). Prophylactic methods (e.g., preventing or reducing the incidence of relapse) are also considered treatment.

In some embodiments, treatment can also involve combination with other existing modes of treatment, for example existing agents for treatment of cardiovascular diseases or disorders, for example but are not limited to diuretics such as furosemide (LASIX®), bumetanide (BUMEX®), hydrochlorothiazide (HYDRODIURIL®), spironolactone (ALDACTONE®), eplerenone (INSPRA®), triamterene (DYRENIUM®), torsemide (DEMADEX®), or metolazone (ZAROXOLYN®), or a combination agent (for example, Dyazide®), Spironolactone and eplerenone; furosemide (LASIX®); Inotropes, such as dobutamine, milrinone, Digoxin (LANOXIN®); Vasodilators and ACE inhibitors; such as captopril (CAPOTEN®), enalapril (VASOTEC®), lisinopril (ZESTRIL®/PRINIVIL®), benazepril (LOTENSIN®), quinapril (ACCUPRIL®), fosinopril (MONOPRIL®), and ramipril (ALTACE®); Angiotensin II receptor blockers (ARBs); such as candesartan (ATACAND®), irbesartan (AVAPRO®), olmesartan (BENICAR®), losartan (COZAAR®), valsartan (DIOVAN®), telmisartan (MICARDIS®), and eprosartan (TEVETEN®); Calcium channel blockers; Isosorbide dinitrate and hydralazine (BIDIL®); isosorbide dinitrate and hydralazine. Nitrates; such as isosorbide mononitrate (IMDUR®), isosorbide dinitrate (ISORDIL®); Hydralazine (APRESOLINE®); Beta-blockers: such as carvedilol (COREG®) and long-acting metoprolol (TOPROL XL®) and Natriuretic peptides such as Nesiritide (NATRECOR®), or B-type natriuretic peptide (BNP) and combinations thereof.

In some embodiments, agents which increase the nuclear form of Creb312 as disclosed herein can be combined with other agent, for example therapeutic agent to prevent and/or treat cardiovascular diseases and disorders. Such agents can be any agent currently in use or being developed for the treatment and/or prevention of a cardiovascular diseases or disorders, where the agent can have a prophylactic and/or a curative effect and/or reduce a symptom of a cardiovascular disease or disorder.

In embodiments, an agents which increase the nuclear form of Creb312 as disclosed herein can be used in combination with medicaments commonly known by person of ordinary skill in the art that are claimed to be useful as symptomatic treatments of ischemia or myocardiac infarction. Examples of such medicaments include, but are not limited to, agents known to increase adenosine, as well as medicaments such as Dobutamine, Cardioplegic, Dipyridamole, Nicorandil, Cariporide, Nitroglycerin and analogues and combinations thereof. Thus, combination treatment with one or more agents t agents which increase the nuclear form of Creb312 with one or more other medical procedures can be practiced. In addition, treatment can also comprise multiple agents which increase the nuclear form of Creb312.

The amount which is administered to a subject is preferably an amount that does not induce toxic effects which outweigh the advantages which result from its administration. Further objectives are to reduce in number, diminish in severity, and/or otherwise relieve suffering from the symptoms of the disease in the individual in comparison to recognized standards of care.

Production of compounds according to present regulations will be regulated for good laboratory practices (GLP) and good manufacturing practices (GMP) by governmental agencies (e.g., U.S. Food and Drug Administration). This requires accurate and complete record keeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

Dosages, formulations, dosage volumes, regimens, and methods for analyzing results aimed at increasing the nuclear form of Creb312, or alternatively increasing cardiomyocyte proliferation or promoting cardiomyocyte survival can vary. Thus, minimum and maximum effective dosages vary depending on the method of administration. Suppression of the clinical and histological changes associated with cardiovascular disease can occur within a specific dosage range, which, however, varies depending on the organism receiving the dosage, the route of administration, whether agents which increase the nuclear form of Creb312 are administered in conjunction with other co-stimulatory molecules, and the specific regimen of administration of the agent which increase the nuclear form of Creb312. For example, in general, nasal administration requires a smaller dosage than oral, enteral, rectal, or vaginal administration.

For oral or enteral formulations for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982.

Examples of solid carriers include starch, sugar, bentonite, silica, and other commonly used carriers. Further non-limiting examples of carriers and diluents which can be used in the formulations of the present invention include saline, syrup, dextrose, and water.

The invention also contemplates an article of manufacture which is a labeled container for providing an agent which increases the nuclear form of Creb312 and/oran nuclear form of Creb312 polypeptide. An article of manufacture comprises packaging material and a pharmaceutical agent contained within the packaging material.

The pharmaceutical agent in an article of manufacture is any of the compositions of the present invention suitable for providing an agent which increases the nuclear form of Creb312 and formulated into a pharmaceutically acceptable form as described herein according to the disclosed indications. Thus, the composition can comprise an agent which increases the nuclear form of Creb312, or alternatively a nuclear form of Creb312 polypeptide or a DNA molecule which is capable of expressing an agent which increases the nuclear form of Creb312, or a DNA molecule which is capable of expressing the nuclear form of Creb312 polypeptide.

The article of manufacture contains an amount of pharmaceutical agent sufficient for use in treating a condition indicated herein, either in unit or multiple dosages. The packaging material comprises a label which indicates the use of the pharmaceutical agent contained therein, e.g., for the treatment of a metabolic disorder, for cardioprotection, or for other indicated therapeutic or prophylactic uses. The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent. In preferred embodiments, the packaging material includes a label that is a tangible expression describing the contents of the article of manufacture and the use of the pharmaceutical agent contained therein.

In some embodiments of the present invention may be defined in any of the following numbered paragraphs:

1. A method for increasing the survival of a cell in response to stress, comprising contacting the cell with at least one agent which increases the level of the nuclear form of Creb312 polypeptide in the cell, such as but not limited to introducing a nucleic acid encoding Creb312(N) into the cell, wherein the nuclear form of Creb312 polypeptide comprises the amino acid residues 1 to 376 of SEQ ID NO: 2 or a biologically active fragment or variant thereof, and wherein the presence or increase in nuclear form of Creb312 polypeptide in the cell increases the survival of the cell in response to stress.
2. A method for inducing the proliferation of a cardiomyocyte, comprising contacting the cardiomyocyte with at least one agent which increases the level of the nuclear form of Creb312 polypeptide in the cardiomyocyte, such as but not limited to introducing a nucleic acid encoding Creb312(N) into the cardiomyocyte, wherein the nuclear form of Creb312 polypeptide comprises the amino acid residues 1 to 376 of SEQ ID NO: 2 or a biologically active fragment or variant thereof, and wherein the increase in nuclear form of Creb312 polypeptide in the cardiomyocyte induces the proliferation of the cardiomyocyte.
3. The method of paragraphs 1 or 2, wherein the agent is an activator of the PI3K/Akt pathway.
4. The method of paragraph 3, wherein an activator of the PI3K/Akt pathway is selected from a group consisting of; a ILK polypeptide or an activator of the ILK polypeptide. The method of paragraphs 1 or 2, wherein the agent comprises a polypeptide of amino acid sequence SEQ ID NO: 3 or a biologically active variant or biologically active fragment thereof.
5. The method of paragraphs 1 or 2, wherein the agent is a nucleic acid or nucleic acid analogue which encodes a polypeptide comprising amino acid sequence SEQ ID NO: 3 or a functional variant or functional fragment thereof.
6. The method of paragraph 6, wherein the nucleic acid or nucleic acid analogue is selected from DNA, RNA, messenger RNA (mRNA), genomic RNA, PNA, pcPNA and Locked nucleic acid (LNA).
7. The method of paragraph 1 or 2, wherein the agent is a protease which cleaves the full length Creb312 polypeptide of SEQ ID NO: 2 at SP1 and/or SP2 cleavage sites.
8. The method of paragraph 1, wherein the stress is mechanical stress.
9. The method of paragraph 1, wherein the stress is stretch stress.
10. The method of paragraph 1, wherein the stress is selected from the group consisting of; hormonal stress, mechanical stress, stretch stress, hypoxic stress.
11. The method of paragraph 1, wherein the cell is a cardiac cell.
12. The method of paragraph 12, wherein the cardiac cell is a cardiac progenitor cell.
13. The method of paragraph 12, wherein the cardiac cell is a cardiomyocytes or cardiomyocyte precursor.
14. The method of paragraphs 2 or 14, wherein the cardiomyocyte is a postnatal cardiomyocyte.
15. The method of any of the above paragraphs, wherein the cardiomyocyte is a human cardiomyocyte.
16. The method of any of the paragraphs above, wherein the cells are present in a subject.
17. The method of paragraphs 2 or 14, wherein the cardiomyocytes are present in a subject.
18. The method of any of the above paragraphs, wherein the cells are from a human subject.
19. The method of any of the above paragraphs, wherein the subject is a mammal.
20. The method of any of the above paragraphs, wherein the mammal is human.
21. The method of any of the above paragraphs, wherein the subject has, or is at increased risk of cardiovascular condition or disease or injury.
22. The method of paragraph 22, wherein the disease is selected from the group consisting of congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, autoimmune endocarditis, and congenital heart disease.
23. The method of paragraphs 1 or 2, further comprising transplanting a cell and/or cardiomyocyte into the subject.
24. A composition comprising a Creb312(N) polypeptide or a biologically active variant or fragment thereof, or a pharmaceutically acceptable derivative thereof for use in the manufacturer of a medicament to increase the survival of a cell to mechanical stress.
25. A composition comprising a Creb312(N) polypeptide or a biologically active variant or fragment thereof, or a pharmaceutically acceptable derivative thereof for use in the manufacturer of a medicament for treatment of a cardiovascular condition, disease or injury to induce the proliferation of cardiomyocytes.
26. The composition of paragraphs 25 or 26, wherein the Creb312(N) polypeptide comprises amino acid residues 1 to 376 of SEQ ID NO: 2 or a biologically active fragment or variant thereof.
27. A composition comprising at least one agent which results in the cleavage of full length Creb312 polypeptide to a Creb312(N) polypeptide or a biologically active variant or fragment thereof, in the manufacturer of a medicament to increase the survival of a cell to mechanical stress.
28. A composition comprising at least one agent which results in the cleavage of full length Creb312 polypeptide to a Creb312(N) polypeptide or a biologically active variant or fragment thereof, in the manufacturer of a medicament for treatment of a cardiovascular condition, disease or injury to induce the proliferation of cardiomyocytes.
29. The composition of paragraph 28 or 29, wherein the agent activates the PI3K/Akt pathway.
30. The composition of paragraphs 26 or 29, wherein the disease is selected from the group consisting of congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, autoimmune endocarditis, and congenital heart disease.
31. The composition of paragraphs 26 or 29, for the expansion of cardiomyocytes.
32. The composition of paragraph 32, wherein the cardiomyocyte is a postnatal cardiomyocyte.
33. The composition of any of paragraphs 25 to 33, wherein the cardiomyocyte is a mammalian cardiomyocyte.
34. The composition of any of paragraphs 25 to 33, wherein the cardiomyocyte is a human cardiomyocyte.
35. A method to promote the survival of a transplanted cell in a subject, comprising;
(a) contracting the transplanted cell with an agent with at least one agent which increases the level of the nuclear form of Creb312 polypeptide in the transplanted cell, wherein the nuclear form of Creb312 polypeptide comprises the amino acid residues 1 to 376 of SEQ ID NO: 2 or a biologically active fragment or variant thereof,
(b) transplanting the cell into a subject
wherein the increase in nuclear form of Creb312 polypeptide in the cell increases the survival of the cell in response to stress.
36. A method to promote the survival of a transplanted cell to mechanical stress, comprising; introducing into a transplanted cell a nucleic acid encoding a nuclear form of Creb312 polypeptide which is operatively linked to a promoter, wherein the nuclear form of Creb312 polypeptide comprises the amino acid residues 1 to 376 of SEQ ID NO: 2 or a biologically active fragment or variant thereof; wherein expression of a nuclear form of Creb312 polypeptide increases the survival of the transplanted cell to mechanical stress as compared to a cell not expressing Creb312(N) protein.
37. The method of paragraph 35, wherein the cell is contacted with an agent before or after transplantation into a subject.
38. The method paragraphs 35 or 36, wherein the cell is a cardiac progenitor cell.
39. The method of paragraph 36, further comprising a transplanting the cell into a subject.
40. The method of paragraph 36, wherein the promoter is a cardiac promoter.
41. The method of paragraph 36, wherein the promoter is a mechanical stress promoter.
42. The method of paragraph 36, wherein the cell is a human Isl1+ cardiac progenitor, or a progeny thereof
43. The method of paragraph 42, wherein the subject is a human subject.

### EXAMPLES

The examples presented herein relate to the methods and compositions to promote the survival of cells, such as cardiomyocytes, to mechanical stretch stress, and to promote the propagation of cardiomyocytes by increasing the level of the nuclear form of Creb312 (Creb312(N)) in the cells or cardiomyocyte. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

RT-PCR, Northern Blot and In Situ Hybridization. In situ hybridizations were performed with the CD1 strain, with noon of the day of vaginal plug detection being defined as E0.5. Whole mount-In Situ Hybridization on embryos or dissected hearts were done as described in (Wilkinson, 1992). The isl1 and mlc2a riboprobes have been previously described (Cai, Evans, Dev Cell, 2002).

For section in situ hybridization, whole embryos were dissected in ice-cold, DEPC-treated PBS, fixed in 4% PFA/PBS at 4° C. and incubated in 25% sucrose/PBS overnight at 4° C. after washing in PBS. After embedding and freezing in OCT cryomatrix (Leica Microsystems Nussloch GmbH, Wetzlar, Germany), embryos were sectioned sagitally at 20 μm. In situ hybridization using digoxigenin-labeled riboprobes was done according to (Wilkinson, Nieto, 1993).

Generation of Creb312 Knockout Mice. Genomic sequences surrounding the basic leucine zipper domain were obtained through screening of a 129/SvJ lambda phage genomic library (Stratagene) and used to clone the targeting vector. The targeting vector contains a cassette with stop codons in three reading frames and a IRES-EGFP reporter cassette as well as a floxed PGK-neo-polyA cassette, that are surrounded by the left and right arms containing more than 10 kb of genomic sequence of the Creb312 locus in total. After successful homologous recombination, the basic leucine zipper domain required for DNA binding and function as transcription factor is replaced by the reporter cassette and floxed PGK-neo-polyA cassette, thus resulting in the formation of a truncated, functionally inactive form of Creb312 in the targeted cells. The targeting vector was electroporated into MPI-2 ES cells, selected with G418 for neomycin resistance. Resistant clones were genotyped for the correct homologous recombination, and ES clones with the correct recombination event were aggregated with CD1 embryos at the morula stage, and transferred into pseudo-pregnant CD1 female mice. The chimeric offsprings were further mated to 129/Sv animals to maintain in a 129/Sv isogenic background or backcrossed into C57BL6 background. The isolation of the Creb312 genomic sequences, cloning of the targeting vector, electroporation into ES cells and generation of the knockout mice were performed at the Max-Planck-Institute (MPI) for biophysical chemistry, Department of Molecular Cell Biology, Goettingen, Germany.

Cloning of Creb312 cDNA. The Creb312 cDNA was cloned by screening a E14.5 brain lambda phage cDNA library (generated in the MPI for biophysical chemistry, Department of Molecular Cell Biology, Goettingen, Germany) with a DNA probe spanning 400 by of the Creb312 cDNA. At the time of the discovery of Creb312 in 2002, this 400 by sequence was the only information available for Creb312 in the ENSEMBL database. The insert sequences of the positive clones were isolated, excised and subcloned to combine them into the complete, 1.5 kb long Creb312 cDNA. The isolation of the Creb312 cDNA was performed at the MPI for biophysical chemistry in Goettingen, Germany.

Isolation of neonatal rat ventricular cardiomyocytes. All procedures described below were conducted according to the guidelines of the Harvard University Animal Care and Use Committee. Ventricular myocytes were isolated from 2 day-old Sprague Dawley rats as briefly described. Excised ventricular tissue was agitated in a 0.1% trypsin solution cooled to 4° C. for approximately 14 hours. Trypsinized ventricles were dissociated into their cellular constituents via serial exposure to a 0.1% solution of collagenase type II at 37° C. for 2 minutes. The myocyte portion of the cell population was enriched by passing the dissociated cell solution through a nylon mesh with 40 μm pores, and then pre-plating twice for 45 minutes each time. 10⁶ isolated myocytes were seeded onto silicon membranes coated with fibronectin in culture medium consisting of M199 base supplemented with 10% heat-inactivated Fetal Bovine Serum, 10 mM HEPES, 20 mM glucose, 2 mM L-glutamine, 1.5 [symbol] M vitamin B-12, and 50 U/ml penicillin. On the second day of culture, the serum concentration of the medium was reduced to 2%, and the medium was changed every 48 hours thereafter.

Adenovirus preparation and infection. Replication-defective recombinant adenoviruses expressing GFP and either HA-Creb312(FL) or HA-Creb312(N) under the control of CMV in bicistronic cassettes were generated using the AdEasy XL system (Stratagene). Briefly, HA-Creb312(FL) or HA-Creb312(N) were subcloned into the adenovirus shuttle vector pAdTrack-CMV (generous gift by Bert Vogelstein, Ref), and these constructs used for the generation of Ad.Creb312(FL) or Ad.Creb312(N), respectively, according to manufacturer's instructions. Adenoviral shuttle constructs for the expression of miRNAs against rat ILK, rat FAK, rat Creb312 under the control of hCMV were obtained by subcloning the XbaI/HindIII fragments of the respective miRNA expression constructs in pcDNA6-GW/EmGFP-miR (Invitrogen) into pShuttle (Stratagene). The recombinant adenoviruses were concentrated and purified by CsCl gradient ultracentrifugation, and the virus titer was determined through plaque assay.

NRVM were transduced with adenovirus at 5 moi (multiplicity of infection) in serum-free M199 for 90 min and maintained for 48 h in M-199/2% FCS before mechanical stretch, hypoxia experiments or treatment with chemicals.

Biaxial mechanical stretch experiments. Freshly isolated NRVM were seeded at 10⁶ on fibronectin-coated silicon membranes and transduced with Ad.Creb312(FL) 48 h later. 2 days later, biaxial mechanical stretch experiments were carried out on devices as previously described (Ref, Andre Kleber paper) for indicated times. Cells were rinsed once in ice-cold PBS, harvested, lysed and processed for immunoblot. For inhibition experiments, NRVM were treated with chemical inhibitors for 30 min prior to stretching. All chemical inhibitors used were from Calbiochem except Akt1/2 inhibitor and wortmannin (Sigma). Concentrations used were: Akt1/2 inhibitor (1 μM), SH6 (10 μM), LY294002 (10 μM), PD98059 (25 μM), U0126 (10 μM), SP600125 (10 μM), PP2 (1 μM), SB203580 (25 μM), wortmannin (200 nM).

Treatment with chemical ER stressors, cardiac hypertrophic agonists, hypoxic stress. 5×10⁵ NRVM were plated in 6 well plates, transduced with Ad.Creb312(FL) and treated 48 h afterwards with tunicamycin (Tm), thapsigargin (Tg), brefeldin A (BFA) or DTT (1 mM) for 30 min and harvested for immunoblot analyses. Alternatively, NRVM were treated with phenylephrine, angiotensin II, endothelin or isoproterenol for 24 h. For hypoxic stress, 5×10⁵ NRVM were plated in 6 well plates and switched from 20% O₂/5% CO₂ to 0.1% O₂/5% CO₂ for the indicated times.

RNAi knockdown of rat ILK, FAK, Creb312. miRNA oligonucleotides directed against the coding sequences of rat ILK, FAK and Creb312 were annealed and cloned into pcDNA6-GW/EmGFP-miR (BLOCK-iT PolII miR RNAi expression vector kit, Invitrogen). The following oligonucleotides were used: rat ILK #1 #2 #3; rat FAK #1: #2: #3; rat Creb312 #1: ATAAGTGTCTGATGAGGAG, #2 (SEQ ID NO: 4): TTAGGAATAAGTGTCTGGCAT (SEQ ID NO: 5), #3: TAGATTAGCAGGTTCCTGGAT (SEQ ID NO: 6). To quantify the knockdown efficiency of individual miRNA sequences, coding sequences of rat ILK, FAK or Creb312 were cloned into psiCHECK-2 vector (Promega) in fusion with Renilla luciferase reporter gene. μg reporter construct were co-transfected with 2 μg miRNA construct into 2×10⁵ COS7 cells, and after 48 h, cells were lysed and luciferase assays performed with Dual-Glo Luciferase Assay System (Promega) according to manufacturer's instructions. Firefly luciferase signal, that is encoded by a separate expression cassette by the psiCHECK2 vector, is used for normalization of the Renilla luciferase signal. The knockdown efficiency of a miRNA sequence correlates with the suppression of the Renilla signal.

Immunoprecipitation and Immunoblot. For immunoprecipitation, 10 μg pcDNA3/3xFlag-Creb312(N) were transfected into COS7 cells with Fugene HD (Roche). 48 h later, cells were washed once with PBS, harvested and lysed in 50 mM Tris-HCl pH 7.4, 150 mM NaCl, 1% Triton X-100 and Complete Protease Inhibitor (Roche) on ice. After centrifugation at 13000 rpm, 30 min, 4C, the supernatant was used for subsequent experiments. Embryonic hearts of various stages were homogenized in ice-cold PBS with a Rotor-Stator Homogenizer, and nuclear extracts were prepared from the cell pellets using the NE-PER Nuclear and Cytoplasmic Extraction Reagents (Pierce). Protein concentrations were determined with the Protein Assay Dye Reagent (Bio-Rad). 1.8 mg 3× Flag-Creb312(N) protein extract and 25 μg heart nuclear extract were rotated with 40 μl anti-Flag M2 Affinity gel (Sigma) at 4C over night. Resins were washed twice each with 50 mM Tris-HCl pH 7.4, 150 mM NaCl with or without 0.1% Triton X-100, eluted with 15 μg 3× Flag peptide (Sigma), and 25 μl or 75 μl eluate were loaded on 10% SDS-polyacrylamide gels for subsequent immunoblot analysis. For immunoblots of mechanical stretch- or chemically treated-cardiomyocytes, 25 μg total cell extract were separated on 10% SDS-polyacrylamide gels. Primary antibodies used were: anti-Flag M2 (Sigma, 1:200), anti-HA (Bethyl, 1:4000), anti-Creb312 (1:2000), anti-phospho (Ser473)Akt (Cell Signaling, 1:1000), anti-phospho (Thr308)Akt (Cell Signaling, 1:1000), anti-Akt (Cell Signaling, 1:1000), anti-phospho p44/42 (Cell Signaling, 1:1000). Secondary antibodies (horseradish peroxidase-conjugated rabbit anti-mouse IgG or goat anti-rabbit IgG, Dako) were used at 1:10⁵′ and immunoblots were developed with SuperSignal West Femto Substrate (Pierce).

Histological Analysis, H3P immunohistochemistry, TUNEL staining. Heterozygous Creb312 mutant mice were mated, and the noon of the day of vaginal plug detection was defined as E0.5. Embryos were dissected in ice-cold PBS, fixed in 4% PFA/PBS over night, dehydrated in ethanol series and embedded in paraffin. Sections were cut at 8 μm. Hematoxylin-eosin stainings were performed according to standard protocols. For immunohistochemistry, sections were deparaffinized, rehydrated in ethanol series and subjected to antigen retrieval in Antigen Unmasking Solution (Vector Laboratories) following manufacturer's instructions. Blocking was carried out in 10% FCS/PBST, and anti-phospho H3 antibody (Upstate) was applied at 1:200 in blocking solution at 4C over night. Sections were washed in PBS and detected with Alexa 488-coupled goat anti-rabbit antibody (Invitrogen) in blocking solution at 1:200. TUNEL staining was performed with the ApopTag Plus In Situ Apoptosis Fluorescein Detection Kit (Chemicon) according to instructions.

### Example 1

### Structure and Subcellular Localization of Creb312

In an in silico bioinformatics screen for novel transcription factors involved in endoplasmic reticulum (ER) stress in heart by using consensus sequences of known DNA binding domains, the inventors identified cAMP response element-binding protein 3-like 2 (Creb312) (SEQ ID NO: 2, encoded by SEQ ID NO: 1), which encodes for a transcription factor of the basic region/leucine zipper (Creb312) family. Bioinformatical alignment to other members of this protein family showed highest homology to Creb3L1/Oasis, Creb3L3, Creb3L4 and ATF6 (see FIG. 1A), that have previously been identified as mediators of the ER stress response and that belong to the Oasis subfamily of Creb312 proteins. Similar to these proteins, Creb312 is a type II ER membrane protein that comprises a N-terminal part containing the characteristic DNA-binding basic region, a nuclear localization signal and the leucine zipper region facing the cytosol, a transmembrane domain as well as the C-terminal part facing the ER lumen (see FIG. 1B). Creb3L1, Creb3L3, Creb3L4 and ATF6 belong to a group of transcription factors that are activated proteolytically in a process described as “regulated intramembrane proteolysis” (RIP). This process was initially identified and best studied in SREBP, a pivotal transcription factor for the regulation of cellular cholesterol and fatty acid metabolism (Brown et al, 2000). Shortly, this mechanism involves the subsequent cleavage of substrate proteins by two proteases, the Site-1 protease (SIP) and the Site-2 protease (S2P), the translocation of their cytosolic portion into the nucleus, where it modulate the transcription of downstream target genes. Like these proteins, Creb312 contains the characteristic recognition sequence (RxxL) (where x is any amino acid) for SIT prevalent in all RIP substrate proteins (Espenshade et al., 1999). This initial protein cleavage appears to be the prerequisite for the subsequent cleavage within the ER membrane by the S2P protease. Although no stringent consensus sequence has been identified for the S2P recognition site so far, Creb3L1 has been shown to be processed by both S1T and S2P similar to ATF6 and SREBP (Murakami, J Neurochem 2006). Thus, the high homology of the amino acid sequences in the transmembrane domains between Creb3L1 and Creb312 (FIG. 1C) strongly demonstrates that the activation of Creb312 in RIP occurred through a similar mechanism. The proteolytic activation of transcription factors in RIP allows rapid cellular response and adaptation to metabolic and physiological stress signals such as dysfunctional lipid homeostasis or accumulation of unfolded proteins in the ER. Herein, the inventors have suprizingly discovered an involvement of RIP in response to mechanical and hormonal stress signals.

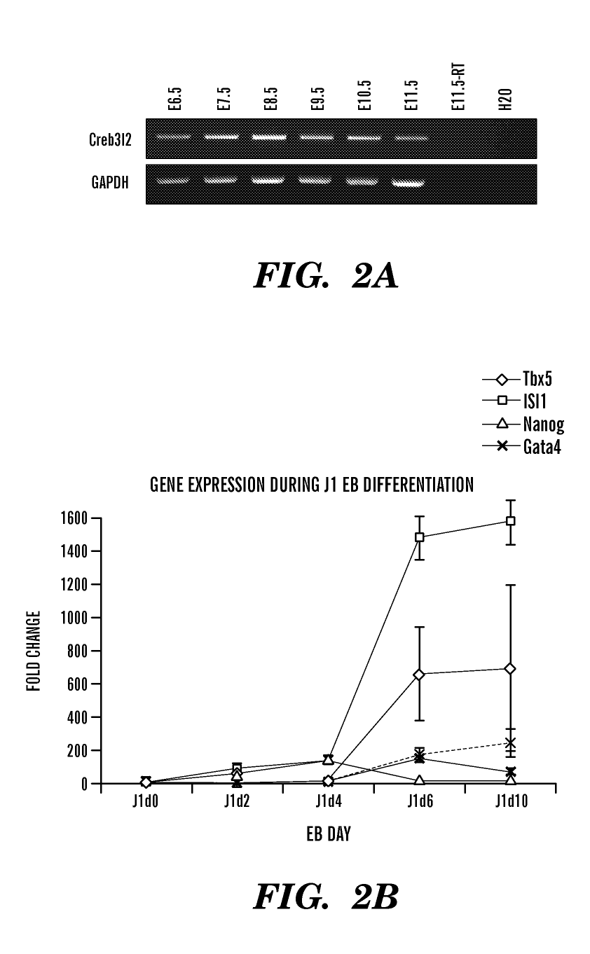

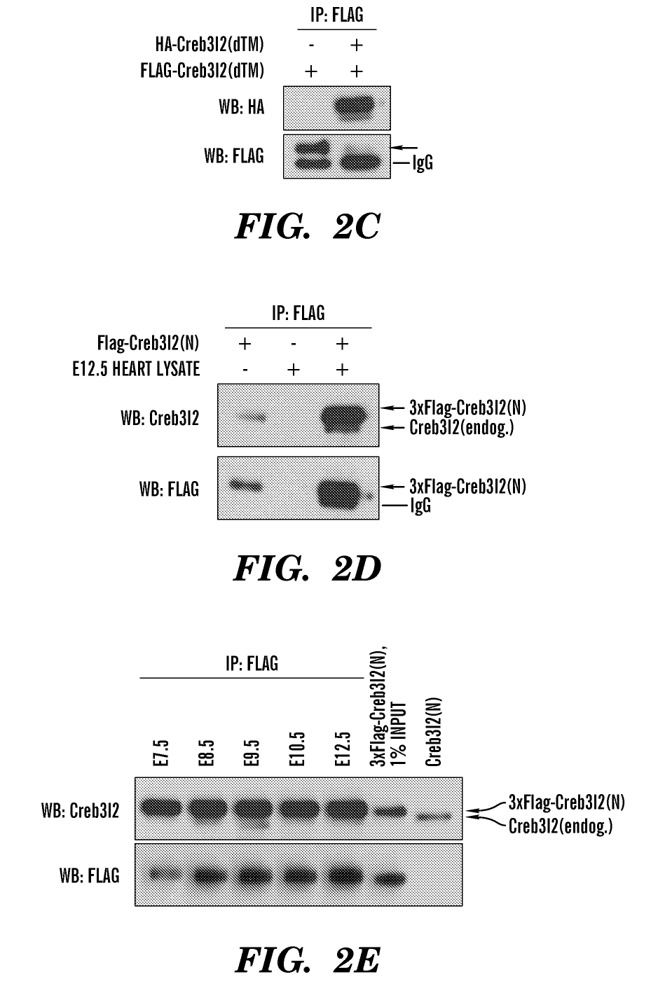

To assess the intracellular sublocalization of Creb312, the inventors transfected Cos7 fibroblasts with plasmid constructs expressing the HA-tagged full-length Creb312 (HA-Creb312FL) and a deletion construct HA-Creb312Δ™ (comprising amino acids 1-376 of SEQ ID NO: 2) lacking the transmembrane domain and the C-terminal portion (see FIG. 2D). Similarly to ATF6, Creb311 and Creb313, HA-Creb312FL is localized in the ER membrane, whereas HA-Creb312(N) is localized exclusively in the nucleus (FIG. 1D), demonstrating that, upon proteolytic cleavage and removal of the transmembrane domain in RIP, the N-terminal portion of Creb312 (amino acids residues 1-376 of SEQ ID NO: 2) indeed translocates into the nucleus and acts as a transcription factor.

Expression of Creb312.

During mouse embryogenesis, creb312 transcript is detectable as of E6.5, which was the earliest stage studied by RT-PCR (FIG. 2A). Whole mount-in situ hybridization demonstrates that the creb312 transcript is initially enriched in the cardiac crescent of the early embryonic day 7.5 (E7.0) embryo (FIG. 2B), that contains cardiac progenitor cells, thus demonstrating that Creb312 is expressed in these cardiac progenitor cells. In order to localize its site of expression in greater detail, the inventors compared its expression pattern with those of isl1 and mlc2a. Isl1 is a marker gene for cardiac progenitors of the secondary heart field, whereas mlc2a is expressed in early differentiated cardiomyocytes (Cai et al., 2003). At E7.5, Creb312 mRNA expression pattern is overlapping with that of mlc2a and isl1 (data not shown). In the linear and looping heart tube stage (E8.0-E9.5), Creb312 mRNA is also highly expressed in the outflow tract, the myocardial wall (data not shown) and the endothelial lining of the heart (endocardium) (data not shown). This demonstrates that Creb312 the gene is expressed in both the primary and secondary heart fields. In addition, Creb312 gene expression is detected in the endocardium and the mesenchyme of the endocardial cushion of the heart, the latter of which will give rise to the atrioventricular valves (data not shown). After E8.5, creb312 is also expressed in the myoblasts of somites (data not shown). In partocilar, using whole mount-in situ hybridization of full length Creb312, the inventors demonstrate that the Creb312 transcript is initially enriched in the cardiac crescent of the early embryonic day 7.5 (E7.0) embryo (date not shown). When the expression of full length Creb312 is compared with the developmental expression pattern Isl1 and mlc2a using whole mount in situ, Creb312 mRNA expression pattern at E7.5 overlaps with that of mlc2a and isl1 (data not shown). At between E8.0-E9.5 Creb312 is expressed in the linear and looping heart tube stage (data not shown), and full length creb312 mRNA is also highly expressed in the outflow tract, the myocardial wall (data not shown). After E8.5, creb312 is also expressed in the myoblasts of somites (data not shown).

At E9.5, Creb312 transcript is detected throughout the whole heart and outflow tract (data not shown). In particular, transverse sections at different levels show that creb312 is expressed in the outflow tract, endocardium, atrium, endocardial cushion and the trabeculae of the left and right ventricles (data not shown).

The onset of Creb312 expression was compared to those of known cardiac marker genes of the primary and/or secondary heart fields, such as nkx2.5, isl1, gata4, ggf10, hand1 and tbx5 (FIG. 2B). Creb312 expression is strongly activated in CJ7 ES cells at EB day 6, parallel to the expression of nkx2.5 and tbx5, marker genes for both the primary and secondary heart fields, and increases until EB day 10. Its expression occurs concomitantly to hand1 and fgf10 as well as isl1, marker genes for the primary or secondary heart fields, respectively (Olson et al.).

Creb312 Homodimerizes and is Proteolytically Cleaved Following the Onset of Heart Beat.

Creb312 gene expression begins at the cardiac crescent stage, as discussed above (data not shown). The inventors then sought to determine whether the presence of Creb312 transcript coincides with the formation of the nuclear, transcriptionally active form of the Creb312 protein. The inventors demonstrated that, by cotransfecting plasmid constructs encoding HA- and/or Flag-tagged Creb312(N) (i.e. cells expressing the N terminal 1-376 amino acids of SEQ ID NO: 2) in COS7 cells and performing immunoprecipitation assays (FIG. 2C), that Creb312(N) homodimerizes in vitro, confirming the bioinformatics that predicts that the amino acid charge distribution on the leucine zipper Creb312 enables it to form homodimers (Vinson et al., 2002). To further investigate the time points at which Creb312 is transcriptionally active during mouse development, the inventors examined the presence of nuclear Creb312 protein in vivo in mouse embryo hearts (FIG. 2D). Flag-tagged Creb312(N) overexpressed in COS7 cells was used to co-immunoprecipitate endogenous Creb312 from nuclear extracts of E7.5 total embryos or E8.5-E12.5 embryonic hearts. Both overexpressed and endogenous Creb312 were then detected with a Creb312-antibody targeted against the N-terminal region. The inventors found that the nuclear, proteolytically cleaved form of Creb312 was clearly detected in the heart after E8.5, but never at E7.5 (FIG. 2E), even when an excess of E7.5 nuclear extract was used. These results demonstrate that, although Creb312 transcript is present in the heart as of E7.5, it is only as of E8.5 that the nuclear form of the protein can be detected. Since E8.5 coincides with the initiation of a physiological stressor, the onset of heart beat, the inventors investigated and demonstrated that the proteolytic cleavage and formation of transcriptionally active Creb312 occurs in response to this signalling cue (data not shown).

### Example 2

### Generation of Creb312 Mutant Mice

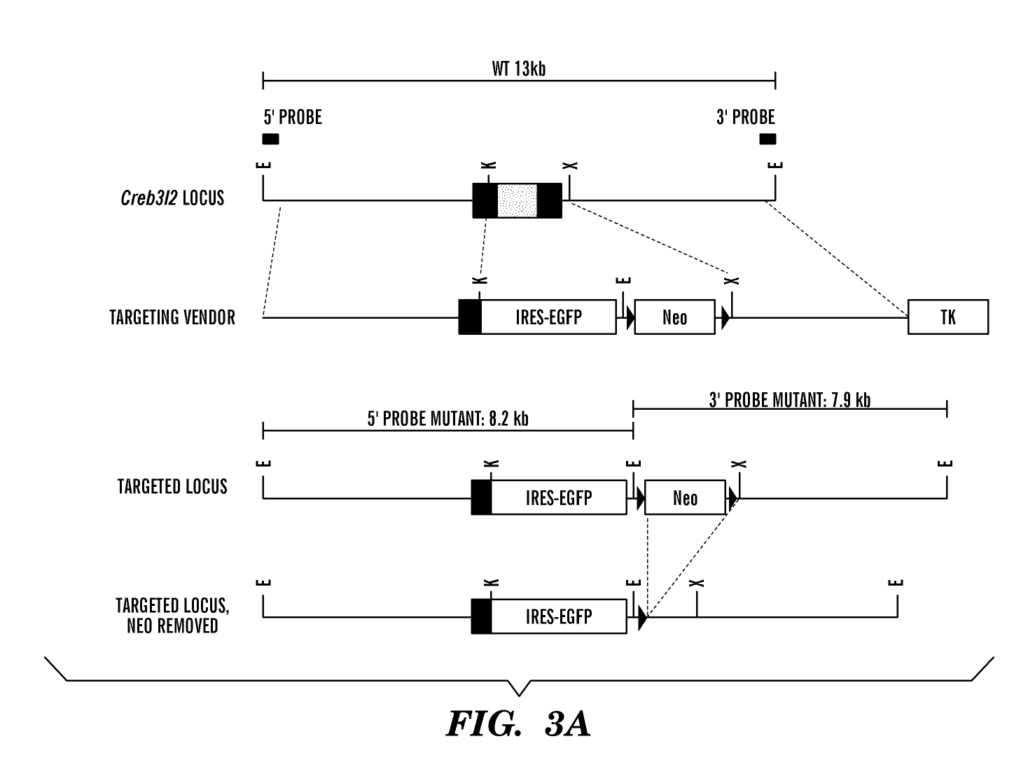

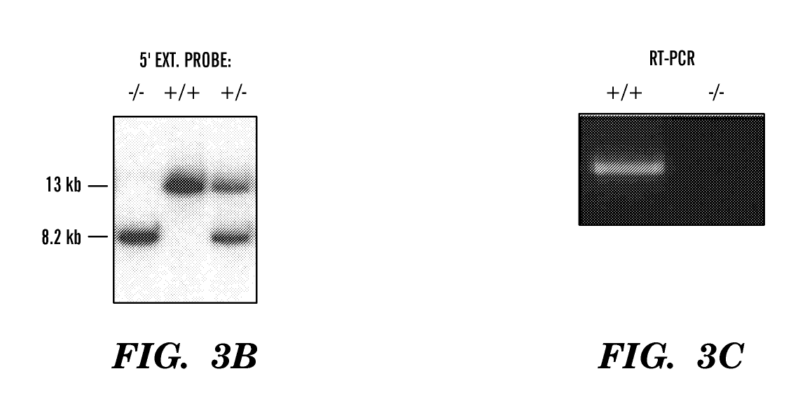

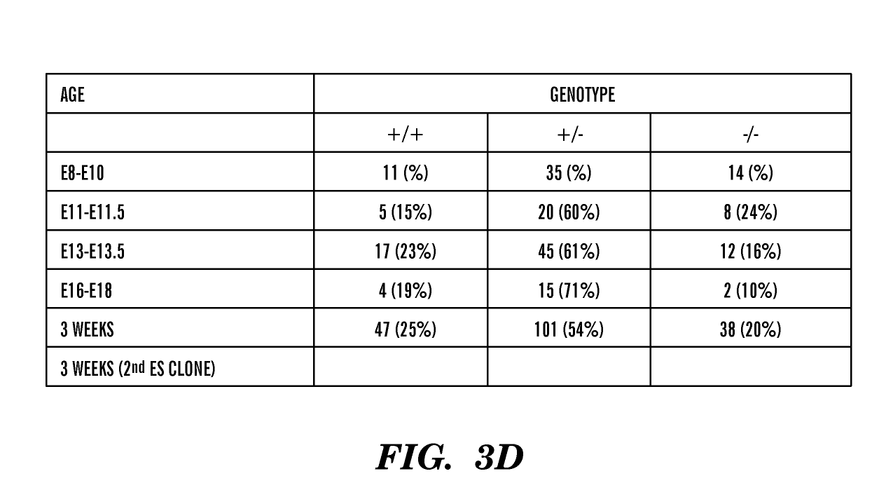

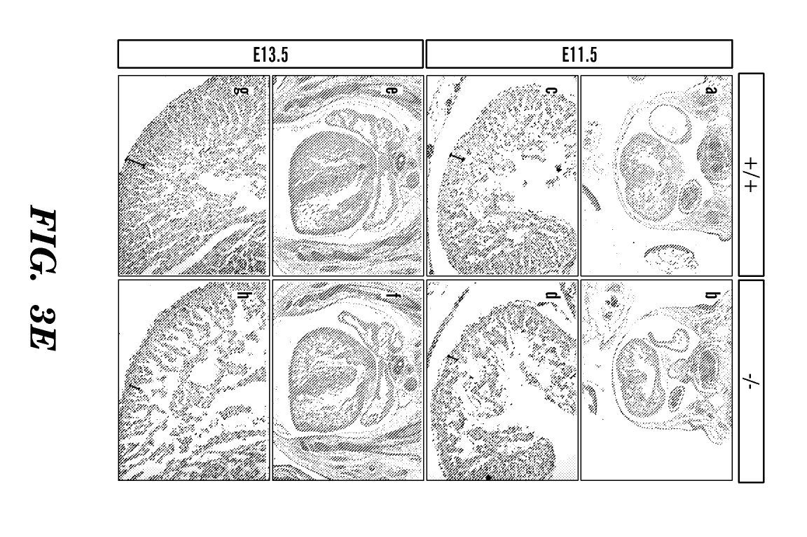

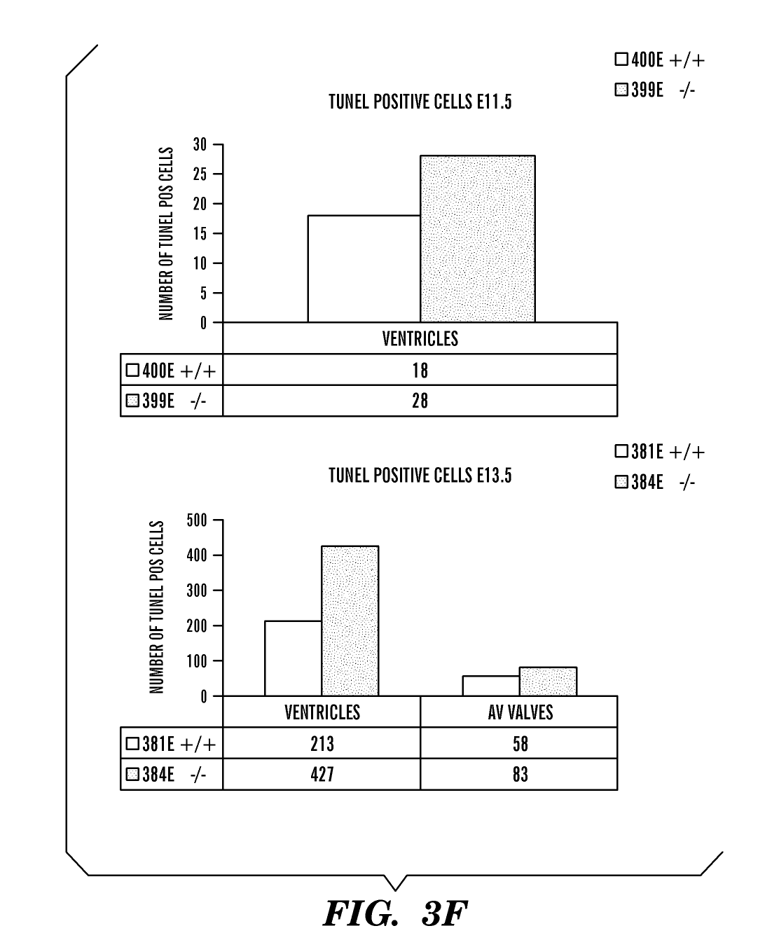

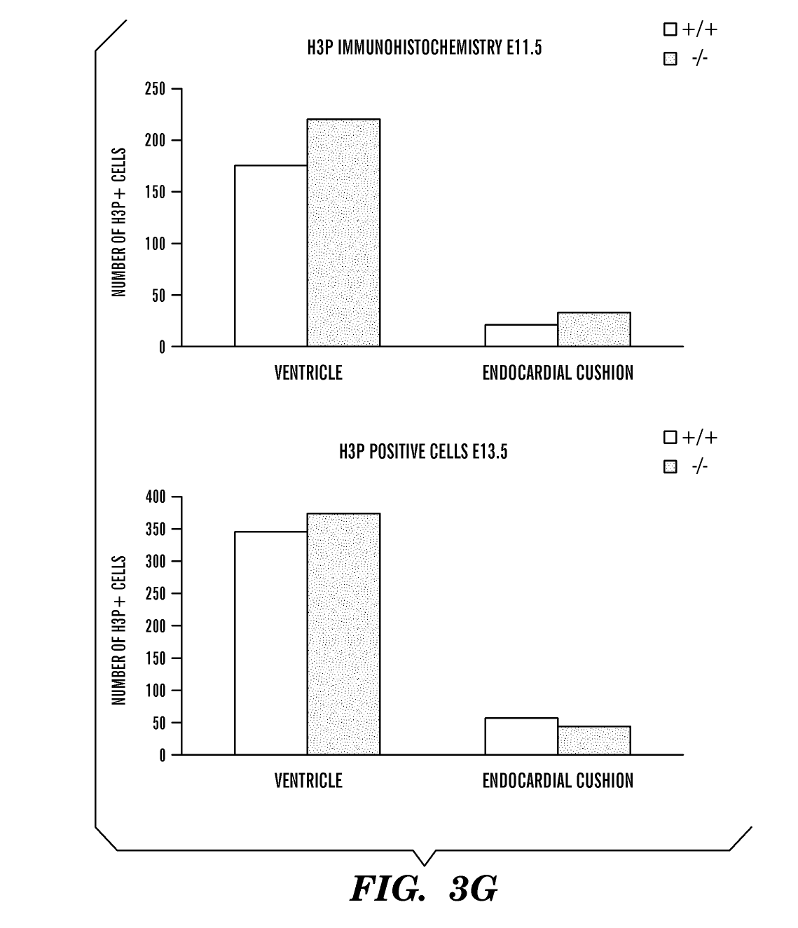

In order to investigate the function of Creb312 in vivo, the inventors generated Creb312 knockout mice through targeted homologous recombination. The DNA-binding basic region, the nuclear localization signal (nls) and the leucine zipper domain required for the dimerization were deleted in the targeting vector and replaced with an IRES-EGFP reporter as well as a floxed neomycin resistance gene cassette (FIG. 3A). Four ES cell clones out of 203 screened G418/gancyclovir resistant clones showed the correct targeting and removal of the Creb312 domain as well as the nuclear localization signal (nls), that are required for the function of Creb312 as a transcription factor, as confirmed by Southern Blot as well as long range PCR on both sides with external probes or primers outside of the targeting arms, respectively (FIG. 3B). In addition, the elimination of functional Creb312 mRNA was confirmed with RT-PCR analysis of RNA from Creb312 null mutant mice hearts (FIG. 3C).

Creb312 heterozygous mice were viable and fertile and maintained in the isogenic 129 background. To generate Creb312 null mutant mice, heterozygotes were intercrossed and the offsprings genotyped at three weeks of age. In the isogenic 129 background, viable homozygous mutants were found at this time with a frequency of 20%, thus deviating from the expected Mendelian ration of 25% (FIG. 3D). Similar results were obtained with mice derived from an independently targeted second ES cell line (data not shown).

To further determine the timepoint of the partial lethality in Creb312 homozygous mutants, embryos of various gestation stages were dissected and genotyped (FIG. 3D). At E8.5-E11.5 the inventors did not detect any deviation from the Mendelian ratio, while a partial embryonic lethality of 20% of the homozygous embryos were found at E13.5. These findings demonstrate the onset of a partial embryonic lethality between E11.5 and E13.5.

Embryonic Cardiac Phenotype in Creb312 Mutant Mice.

Before E10.5, the inventors did not detect any phenotypic abnormalities in Creb312−/− embryos. After E11.5, although no gross structural abnormalities such as ventricular septal defects (VSD), valve or outflow tract defects were observed in histological examinations, the right ventricular wall of Creb312 null mutant embryos were thinner and hypocellular (FIG. 3E, a-d). This phenotype was even more obvious after E13.5 (FIG. 3E, e-h), where the compact zone of the right ventricle appeared significantly thinner. No obvious thinning was observed in the left ventricular walls, although we do observe the consistent presence of disintegrated cardiomyocytes.

To assess whether the hypocellularity of the right ventricular compact zone was due to increased apoptosis of ventricular cardiomyocytes or due to decreased cell proliferation, the inventors performed TUNEL staining (FIG. 3F) and anti-phospho H3 immunohistochemistry (FIG. 3G), respectively, on histological sections. At E11.5, the inventors detected a 50% increase in the number of TUNEL-positive cells in creb312 null mutant hearts compared to the hearts of wildtype littermates. At E13.5, an even higher (100%) increase of TUNEL-positive cells was observed. However, the inventors did not detect a significant increase of TUNEL-positive cells in the AV valves, consistent with the discovery that observation of the absence of abnormal valve phenotypes. In contrast, no significant difference in the number of phospho-H3-positive cells was detected when comparing ventricular cardiomyocytes or endocardial cushions of wildtype and mutant hearts at either E11.5 or E13.5 (FIG. 3G).

### Example 3

### Proteolytic Cleavage of Creb312 by Biomechanical Stretch Stress, Hormonal Agonists and ER Stressors, but Not by Hypoxia

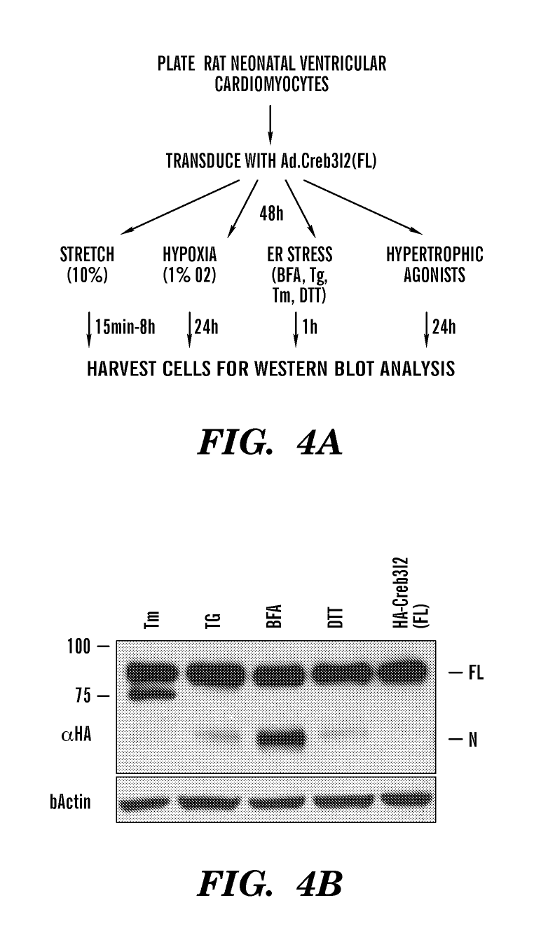

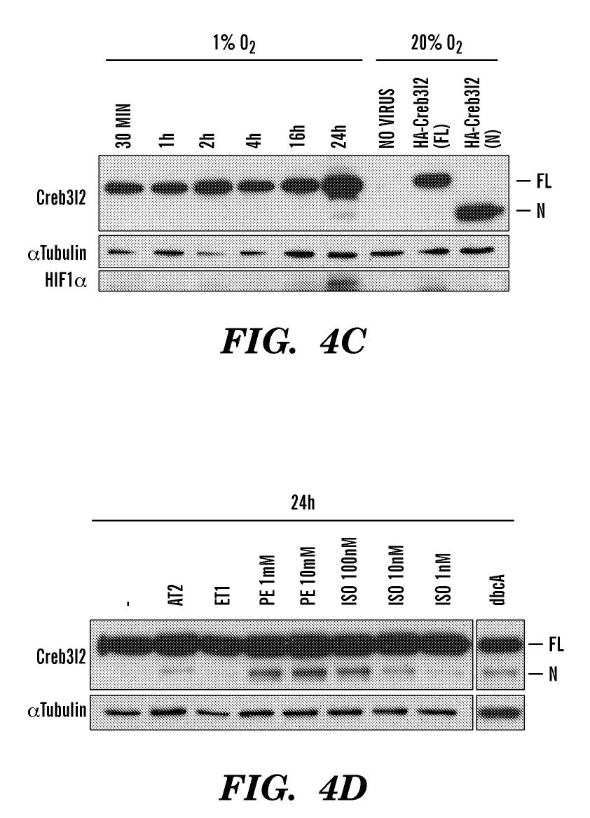

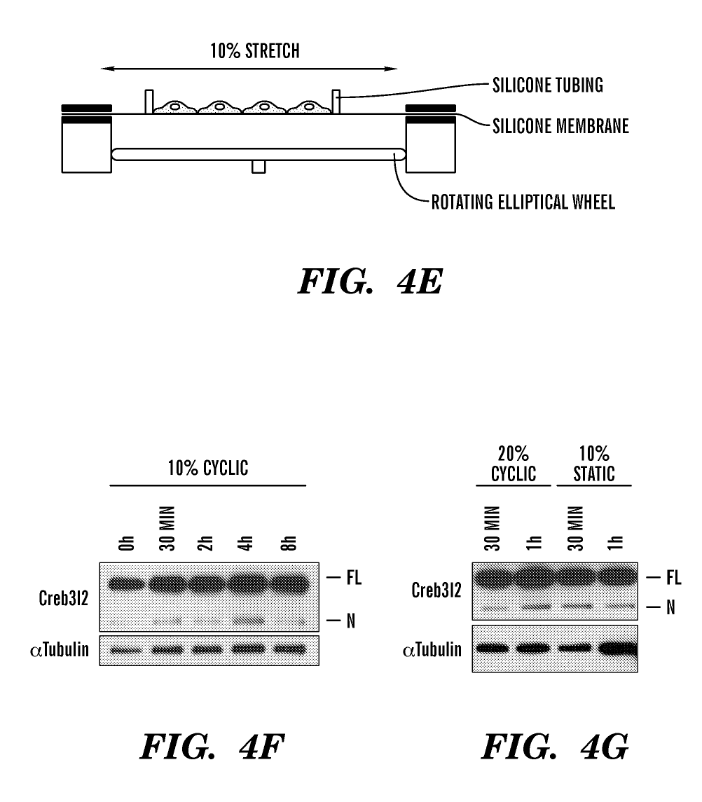

Since several Creb312 proteins closely related to Creb312 (such as Creb311/Oasis and CrebH) have been shown to be involved in the response to ER, ischaemic or inflammatory stress, the inventors investigated which stress signals or stimuli lead to the proteolytic cleavage and formation of the nuclear form of Creb312 in cardiomyocytes. To test various potential stress factors that cardiomyocytes have been implicated to be exposed to, the inventors transduced primary rat neonatal ventricular cardiomyocytes (NRVM) with recombinant adenovirus expressing the HA-tagged, full-length Creb312 (Ad.Creb312(FL)) and subjected them to chemical, hormonal, mechanical and hypoxic stress (see schematic in FIG. 4A). The formation of the proteolytically cleaved, nuclear form of Creb312 was monitored by Western Blot with anti-HA antibody. Treatment with known ER stress-inducing chemicals such as brefeldinA (BFA) and thapsigargin (TG), and to a lesser extend DTT and tunicamycin (Tm) caused proteolytic cleavage of Creb312 (FIG. 4B) within one hour, thus confirming that Creb312, like ATF6, Oasis and CrebH, is responsive to ER stress. Conversely, hypoxia induced the proteolytic cleavage of Creb312 only after 24 h, thus representing a slower response to hypoxic stress. The induction of the cellular response to hypoxic stress in the NRVM was confirmed by the detected translational upregulation of the marker gene HIF1-alpha (FIG. 4C).

To investigate whether Creb312 activation is mediated by hormonal stress stimuli, NRVM were treated for 24 h with hypertrophic agonists such as isoproterenol (ISO) (81/2 adrenergic agonist), phenylephrine (PE) (cd adrenergic agonist), angiotensin-2 (AT2) or endothelin-1 (ET1) (GCRP receptor agonist) (FIG. 4D). Proteolytic cleavage of Creb312 is most strongly induced by isoproterenol and phenylephrine in a dosage-dependent manner, and to a decreasing extend by angiotensin-2 and endothelin-1. To assess whether the isoproterenol-dependent proteolytic cleavage is also cAMP-dependent, NRVM were treated with dbcA, a chemically stabilized, cell membrane permeable form of cAMP. The inventors discovered that nuclear Creb312 is formed in response to cAMP treatment, demonstrating that the activation through isoproterenol (ISO) occurs through the cAMP-dependent pathway (data not shown). Next, the inventors subjected NRVM to 10% or 20% pulsary (cyclic) biaxial mechanical stretch for various time periods, (FIG. 4E) (Kleber paper). The inventors interestingly demonstrated that mechanical stretch stress induced proteolytic cleavage of Creb312 in a time- and stretch intensity-dependent manner, with nuclear Creb312 being first detectable 15 minutes after stretch initiation (FIG. 4F). To distinguish the response to pulsary (e.g cyclic) or static mechanical stretch, the inventors compared the response of transduced NRVM subjected to 10% pulsary (cyclic) stretch versus 10% static stretch. Surprisingly, static mechanical stretch also induced proteolytic cleavage of Creb312, demonstrating that Creb312 responds to mechanical as well as shear stress (FIG. 4G). This discovery was further confirmed through immunocytochemical staining of Ad.Creb312(FL)-transduced NRVM before and after 10% pulsary stretch (data not shown). In particular, double immunocytochemical staining with anti-HA and anti-calnexin of NRVM cells expressing HA-Creb312(FL) following 10% static biaxial mechanical stretch showed anti-HA staining (e.g. Creb312(N)) in the nucleus after 10% static biaxial mechanical stretch (data not shown). Thus, the inventors have discovered that, like other Creb312 members of the Oasis family, Creb312 is an ER stress-responsive transcription factor. In addition, however, Creb312 showed a timewise differential response to various physiological stress signals. In particular, the inventors have discovered that while nuclear Creb312 is formed rapidly in response to ER stress or mechanical stretch stress (within 1 h to 15 min, respectively), they have also discovered that slower response of nuclear form of Creb312 is formed in response to hormonal (cardiac hypertrophic agonists) or hypoxic stress.

### Example 4

### Creb312 Activation by Mechanical Stretch Stress is Mediated by the ILK Pathway and Modulated by PI3K/Akt

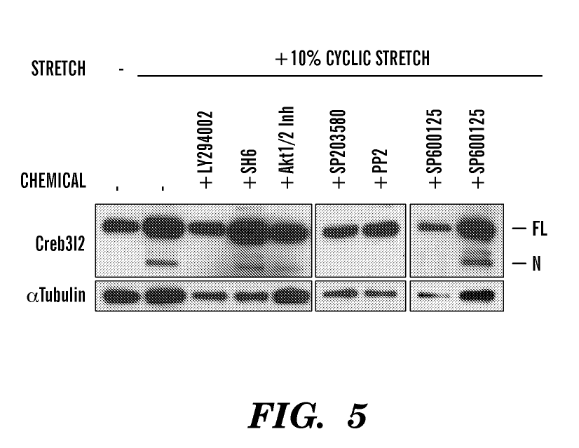

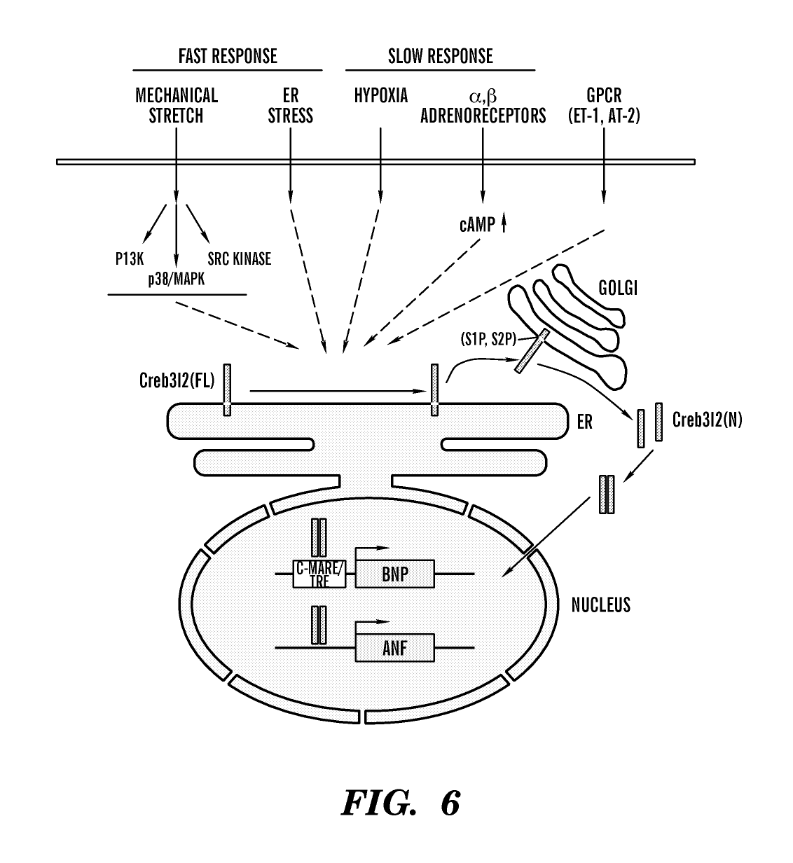

Since the inventors have discovered the cleaved, nuclear form of Creb312 in NRVM after mechanical stretch stress and in the embryonic heart after the onset of heart beat, the inventors investigated which signalling pathway conferred the proteolytic cleavage of Creb312. Since several major signalling transduction pathways such as Erk1/2, JNK, Src kinase and PI3K/Akt have been shown to be activated upon mechanical stretch stress, the effect of various chemical inhibitors of these pathways was tested. The inventors treated Ad.Creb312(FL)-transduced NRVM with the chemicals prior to subjecting them to mechanical stretch stress and examined the formation of the cleaved, nuclear form of Creb312 through immunoblots. Inhibitors against the Erk1/2 (PD98059, U0126), JNK (SP600125), Src kinase (PP2), p38/MAPK (SB203580) did not show any effect on the formation of nuclear Creb312. However, the inventors discovered that PI3K inhibitors LY294002 and wortmannin completely inhibited the proteolytic activation of Creb312, demonstrating that the PI3K pathway is involved. The protein kinase PKB/Akt is an important downstream target for PI3K in cardiomyocytes, in particular in the anti-apoptotic cellular response to physiological stresses. The inventors discovered a complete ablation of Creb312 activation after treatment with Akt inhibitors SH6 and Akt1/2, demonstrating that Creb312 is proteolytically activated in response to signals modulated by the PI3K/Akt pathway (FIG. 5). Thus, the inventors have discovered that agents which activate Akt and/or PI3K are useful to activate Creb312 to produce the nuclear form of Creb312.

Furthermore, RNAi knockdown of ILK leads to reduction of Creb312 activation (data not shown), demonstrating the ILK pathway (where the ILK polypeptide corresponds to SEQ ID NO: 7) is involved. Accordingly, the inventors have discovered that agents that can activate the ILK protein, or administration of the ILK protein corresponding to SEQ ID NO: 7 to induce nuclear form of Creb3L2 is useful in the methods and compositions of the present invention.

Accordingly, the inventors have discovered that Creb312 is a Protease-Activated, Sarcoplasmic Reticulum Transcription Factor triggered by mechanical stress. The inventors have further discovered that Creb312 plays a pivotal role in mechanical stress induced survival pathways in the biology of cardiovascular progenitors and their transition to beating cardiomyocytes. Accordingly, the methods of the present invention can be used to prevent cardiomyopathy and the loss of cardiomyocytes as a response to mechanical stress induced survival cues in heart progenitors and their differentiated progeny.

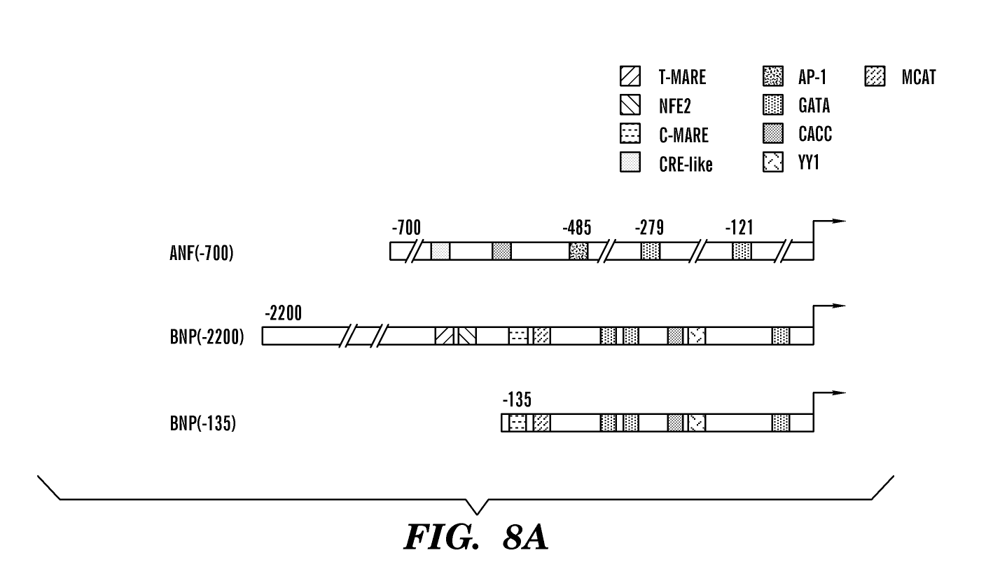

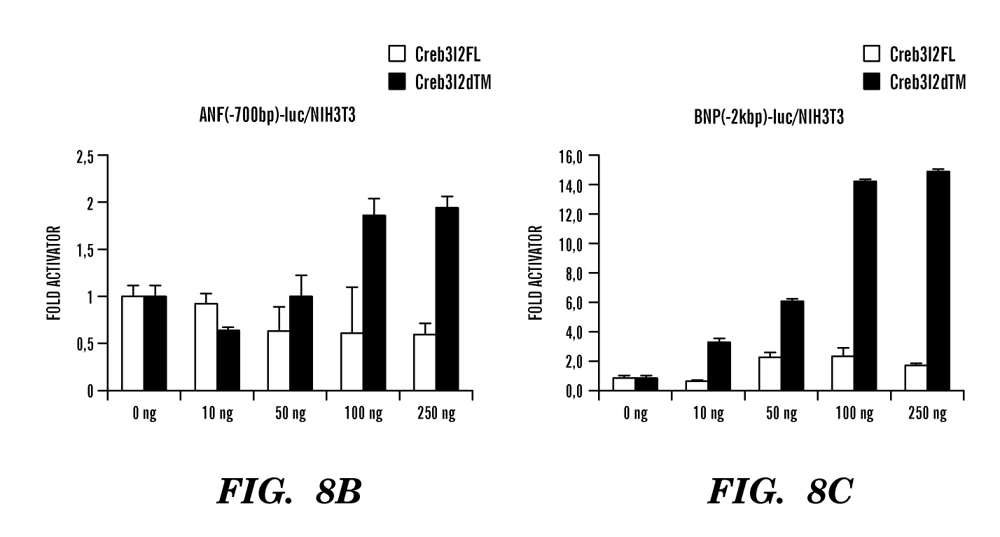

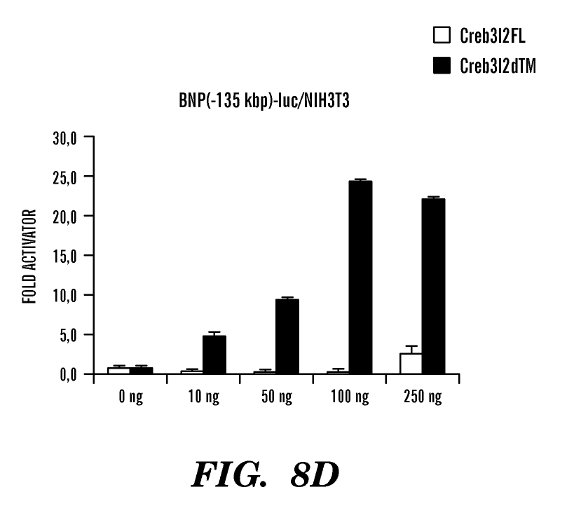

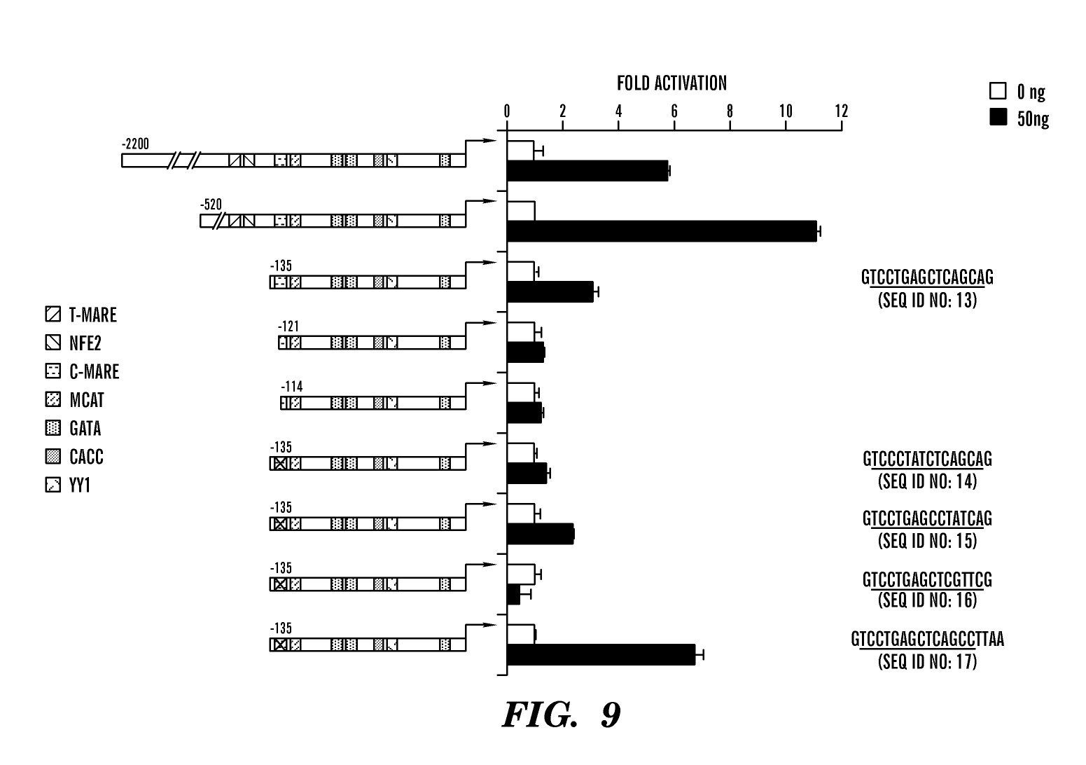

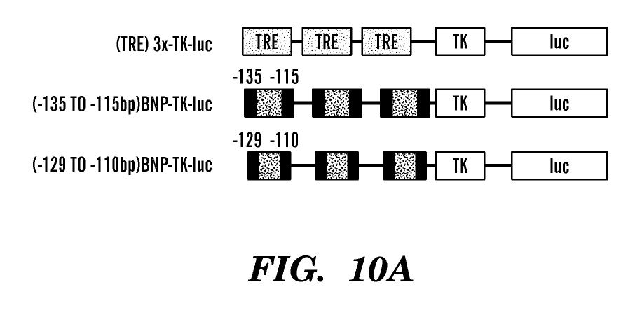

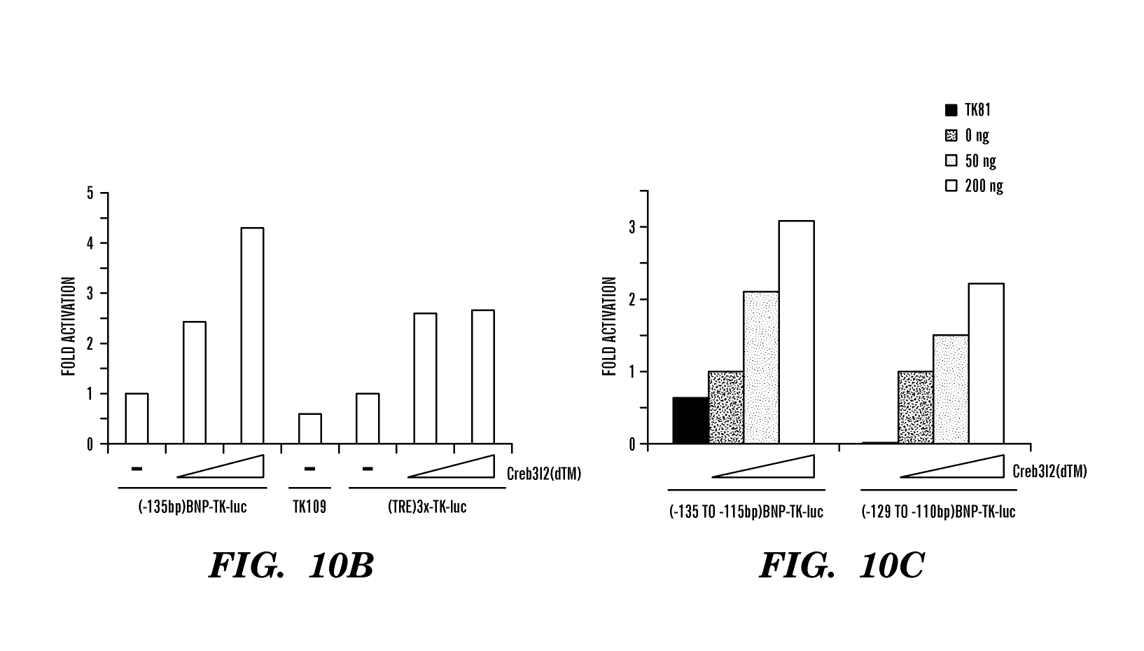

The inventors have discovered that creb312 has a cytoprotective role during mechanical stretch stress, as shown by RNAi knockdown of Creb312 in NRVM model and during mechanical stretch leads to more TUNEL positive cells. Conversely, the inventors demonstrate that by overexpressing Creb312 by adenovirus expression (Ad.Creb312(N)) there are less TUNEL positive cells, demonstrating the ability of Creb312 as a cytoprotection factor during mechanical stretch stress. The inventors also demonstrate that Creb312 functions to regulate the expression of some anti-apoptotic downstream genes, for example CHOP, ANF, BNP, other downstream targets of Akt/PI3K (see FIGS. 7-10).

### Example 5

### Expression of Creb312(N) in Human Isl1+ primordial Progenitor Cells or Post-Mitotic Cardiomyocytes and Transplantation for Treatment of Myocardial Infarction

The inventors demonstrate ex vivo manipulation of postmitotic cardiomyocytes to introduce nucleic acid expressing the Creb312(N) polypeptide, which are then transplanted into a mouse model of myocardial infarction. The cardiomyocytes were transfected with an expression vector comprising nucleic acid encoding Creb312(N).

Similarly, the inventors demonstrate ex vivo manipulation of human Isl1+ primoridial cardiac progenitors which comprise a nucleic acid sequence expressing the Creb312(N) polypeptide, which are then transplanted into a mouse model of myocardial infarction. The Isl1+ primordial cells (as disclosed in U.S. Provisional applications 61/185,752 and 61/256,960, which are incorporated herein in their entirety by reference) were transfected with a leniviral vector comprising nucleic acid encoding Creb312(N) operatively linked to an inducible promoter so that the expression of Creb312 could be regualated by the presence of a drug, e.g. doxycycline.

Transfection of HL-1

Immortalized cardiomyocyte HL-1 cell line (W. Claycomb, LSU Health Sciences Center, New Orleans) were transfected with pcDNA3/Creb312-N or empty pcDNA3. 48 h later, cells were treated with known apoptotic inducers staurosporin or TNFalpha for 24 h and 48 h. Apoptosis was assayed on through AnnexinV staining (BD Biosciences) and flow cytometry and analyzed with FlowJo software (Becton Dickinson).

ES Cell Lines

Nkx2.5-Cre^{+/-}; Rosa-YFP^{+/-} and AHF-Cre^{+/-}; Rosa-YFP^{+/-}; Rosa-rtTA-M2^{+/-} ES cell lines were derived from crosses between Nkx2.5-Cre (Robert Schwartz) and Rosa-YFP or AHF-Cre (Brian Black), Rosa-rtTA-M2 (Konrad Hochedlinger) and Rosa-YFP mice, respectively. Those ES cell lines with the highest differentiation efficiency into cardiac progenitors were stably transduced with lentivirus allowing inducible expression of Creb312-N after doxycycline treatment. YFP-positive Nkx2.5-expressing cardiac progenitor cells were obtained through hanging drop differentiation of the ES cell lines and purified through FACS sorting.

Myocardial Infarction and Cell Transplantation

Male 8-10 week-old NOD.SCID mice (NCI Fredericks) were subjected to myocardial infarction by ligation of the left coronary artery. In a second group, animals without prior MI were used for cell transplantation. Immediately after the MI, 2−5×10⁵ sorted YFP+ cardiac progenitor cells were injected into the border zone of the infracted heart. Animals were treated with doxycycline in drinking water to induce expression of Creb312-N in the grafted cells; as negative control, animals were not treated with doxycycline.

### REFERENCES

The references cited herein and throughout the application are incorporated herein by reference.
Cai, C. L., Liang, X., Shi, Y., Chu, P. H., Pfaff, S. L., Chen, J. et al., 2003. Isl1 identifies a cardiac progenitor population that proliferates prior to differentiation and contributes a majority of cells to the heart. Dev Cell 5, 877-889.
Vinson, C., Myakishev, M., Acharya, A., Mir, A. A., Moll, J. R., Bonovich, M., 2002. Classification of human B-ZIP proteins based on dimerization properties. Mol Cell Biol 22, 6321-6335.
Wilkinson, D. G., Nieto, M. A., 1993. Detection of messenger RNA by in situ hybridization to tissue sections and whole mounts. Methods Enzymol 225, 361-373.

### SEQUENCES

| SEQ ID NO: 1: - the nucleic acid sequence of human Creb312, | |
|---|---|
| (RefSeqID No: NM_194071, GeneID No: 64764, Accession No: AJ549092). | |
| Creb312 is also known as aliases; Creb3L2, cAMP responsive | |
| element binding protein 3-like 2, BBF2H7 and TCAG_1951439. | |
| 1 | ctgggtcctg gagcagagcc gaggagccct ggggtccctc aaagtttgtg tctggagccg |
| 61 | tagcggcaag tgggcttgcg gctaagggat tttcctggga tgagagcggg tcttctgcct |
| 121 | tcattttgga tgcacatccc gctttagccc cggcagcctt tggtccggct cgtgtccctg |
| 181 | gggattctcg gatctccgag gacaccggac gggagcgctt ggccatcctc tctccggcag |
| 241 | aggagcagac gtttgctttc caagtgcaaa actacagaca cgcgcgcgca cacacgcaag |
| 301 | cacacgcgga gagagaggaa ccttgccggt ccgaggcagc tctgcgcgtc ccctcctgcg |
| 361 | cttagcatcc tcggcccagc gcggcccgca ccgccatgga ggtgctggag agcggggagc |
| 421 | agggcgtgct gcagtgggac cgcaagctga gcgagctgtc agagcccggg gacggcgagg |
| 481 | ccctcatgta ccacacgcac ttctcagaac ttctggatga gttttcccag aacgtcttgg |
| 541 | gtcagctcct gaatgatcct ttcctctcag agaagagtgt gtcaatggag gtggaacctt |
| 601 | ccccgacgtc cccggcgcct ctcatccagg ctgagcacag ctactccctg tgcgaggagc |
| 661 | ctcgggccca gtcgcccttc acccacatta ccaccagtga cagcttcaat gacgatgagg |
| 721 | tggaaagtga gaaatggtac ctgtctacag acttcccttc aacatccatc aagacagagc |
| 781 | cagttacaga cgaaccaccc ccaggactcg ttccgtctgt cactctgacc atcacagcca |
| 841 | tctccacccc gttggaaaag gaggaacctc ctctggaaat gaacactggg gttgattcct |
| 901 | cgtgccagac cattattcct aaaattaagc tggagcctca tgaagtggat cagtttctaa |
| 961 | acttctctcc taaagaagcc ccagtggacc acctgcattt gccgcccacc cctccgagca |
| 1021 | gtcacggcag tgactcagag ggcagcctga gtcccaaccc acgcctgcac cccttcagcc |
| 1081 | tgcctcagac ccacagcccc tccagagctg caccccgggc cccctccgcc ctctccagct |
| 1141 | cccctctcct cacggctcct cataaactgc agggatcagg ccctctggtc ctgacagagg |
| 1201 | aggagaagag gaccctgatc gctgagggct atcccatccc caccaaattg cccctgtcaa |
| 1261 | aatcagagga gaaggccctg aagaaaattc ggaggaagat caagaataag atttctgctc |
| 1321 | aggaaagtag gagaaagaag aaagaataca tggacagcct ggagaaaaaa gtggagtctt |
| 1381 | gttcaactga gaacttggag cttcggaaga aggtagaggt tctagagaac actaatagga |
| 1441 | ctctccttca gcaactccag aagcttcaga ctttggtgat gggcaaggtt tctcgaacct |
| 1501 | gcaagttagc tggcacgcag actggcacct gcctcatggt tgtggtgctg tgctttgccg |
| 1561 | ttgcattcgg cagcttcttt caaggctacg ggccctatcc ttctgccacc aagatggctc |
| 1621 | tgcccagcca gcattccctg caggagccct acacagcctc cgtggtgaga tccagaaacc |
| 1681 | tgctgatcta cgaggaacat tctcccccag aggagtcatc cagcccgggc tcggctgggg |
| 1741 | agctgggggg ctgggataga ggttcctccc tgctcagggt gtcagggctg gagtccaggc |
| 1801 | cggatgtgga tcttccccat ttcattatct cgaatgagac cagcctggag aagtcagtgc |
| 1861 | ttttggagct gcagcagcac ctggtcagcg ccaaactgga ggggaatgaa acactaaaag |
| 1921 | ttgtagaact cgacagaaga gtgaacacca ctttctaaag aggctgcctg caccccctcc |
| 1981 | ctttccctta actctacttt tacatcccca aaccaccttt gtcatcagct tttcctcttt |
| 2041 | gccactggat cttcatggag acatgggcaa gcattagtgg cttcagattg gagaccagcc |
| 2101 | tgggacttcc ctgcagtgag agagcatctc cccctggtcc atgcccctcc tgtgcagaag |
| 2161 | ggagcctgca tccctccctt cctttctctt actgccatag gaaattattt taggggttgg |
| 2221 | aggtgggaca agcaggcttg tttccaccaa tagtgccaaa aagatattgc ctaatgtgca |
| 2281 | cctgtgaggt gtaacccccc gctttggaga cgagatggct cttgttcagt caagacccca |
| 2341 | gactctggcc acaaaaatgc cataatgcct gttggtattt ggcaaagcac tgacccgtgt |
| 2401 | cctccgttgc tcgcactggg gtctctggtg tgaacacccc cgacagcagc cctccgccca |
| 2461 | ctctgccccc tgggagccct cgctggatcg tctcgtctcc tgcagcagca ctggcaggcg |
| 2521 | agggctctcg ttcatattct caggccgcaa gtgcaatgcc tgaggggatc aggcttttct |
| 2581 | actccaggca aacctgcccc atcttgtcgc ttttaggacc tcccacaacc tggttcccca |
| 2641 | cacatccata gttctgcctc cccagcttct cctccccagt tgtaaatagt atttattagc |
| 2701 | ttgccgaggc ttcctgctag caaccacact gaagagatcg atgcctcctt tcaagctagc |
| 2761 | caagttttct gcgagccttc agagctagga gggcacccta ggctctggga tcccgtgtct |
| 2821 | ttccagacaa tgttttgttt cctttccttt gttttttctt ttaactggaa taattaccat |
| 2881 | tgaaaaagaa gttcctttga gcatgtatgt gtctgcctct aggatgagct cagagcgaga |
| 2941 | gatgacacaa tgcctcactc aggccccggg ctccctggcc acaagctttt tctatcctgt |
| 3001 | tttcatgaca gagaagggga agccctgttc tgacaacaga catttcagac aaccttgctg |
| 3061 | gctttccaca cctgcctggc cccctcctcc ctccacactt ccactttgtc ctcctcgtcc |
| 3121 | cctacctcaa caaagcaggg tggggtaggt gacatttgtg tatccacatt cttacctttg |
| 3181 | gtagtcaggt ttggctactt tgcagctcgc ccaaagagat acaacctaat ccccaaccta |
| 3241 | cttttagttt ttttgttttt tttttatggt taaaagtaac ttttgtagtt taaaaaaatc |
| 3301 | tttcctcttt catataaata agaagtggaa attgcctttt tattgtgtaa tgtagaaaac |
| 3361 | cctcaagtgt tttttccgag cttgggaaag attttgtgta ggaaatgtgc atagagtttg |
| 3421 | tattttattt ttattagcag ctgaaatgcc tttggttttg gcttctctct ctccctctct |
| 3481 | ctctctgtct ctccttctct ctctcccccc accacccacc cccacacacg tcatctgcat |
| 3541 | tgttattgga gcctgtactt agagggatta agcccacacc ctggcttcca ttccatatca |
| 3601 | ggtacaggat ttgatgttat taacatttgt cgtcatacct cataagtcgg tccctgcctt |
| 3661 | gtctgtctag gcccatttgg ggctccctgt gagtgattcc cctctctctg ctatgctgga |
| 3721 | gacggttcca gcctggaaag cggccaagtt catcttctca ctgtgagtgg aagctggatc |
| 3781 | gggcccccgt agtcctggca gccctgttgt ctggagggtt cttgttgtcc ctcccattag |
| 3841 | ccagggcgga gactgtctga gctgtgcagg aggagggttg ctagtaggtt ctgcttctgc |
| 3901 | ttctctctgc tccactgtct gcagcccaga tcctgttggg cctggctggt gtctggtaac |
| 3961 | catgggcctc cactgaccca tccctctctt ttaaactgtc aggtcattat caggcatagg |
| 4021 | cagcctatag ggcccaaaga aggcaaaaag ataagattta ctcaagtagc atttgggcaa |
| 4081 | tgaggaagga aaggtttcaa atttaggggc agaagtgaga gaatgagcca acccatgtac |
| 4141 | ctgctgcaac tgaaccagac tgggttttca aggctcccag acgtagagta ggaaacgtgc |
| 4201 | tcttctaaat gaggagggag aagataaagg aaacttctag cccctgtcct tagtgctttg |
| 4261 | aggattttat tttctccctt actacgcttg cttgacgtca ctctctctcg acctccaaac |
| 4321 | agcaggactc tttctctggg aaaccatcct tccaaaacgg aatctatgta gacaatggga |
| 4381 | cgttaggcag agagctcaga tggccctttt aagggggctc caagaaccaa catcactgct |
| 4441 | cttttagata aacctctgcc ctccactcct tgcttgagtg ggttaaagga actaacagtt |
| 4501 | gtccctttag gaggacaaaa tggggtcaag aggacacaga agagttgtat agcaccagat |
| 4561 | tggttccaaa tagttaatgg atgtgtgcac attttctgtt cagggattaa gaccagaata |
| 4621 | tcagtggatt tgttttcccc accaagtggc ctcttagact agtcattaac ttatgattag |
| 4681 | ctctaaagat ttcaaatagt ggcagacagt gtcttctgaa tgtaagtttt gagaaatacg |
| 4741 | agtctgtcag agcggccata agccataaag agtcaatctc ttaattatat ttttcatcat |
| 4801 | gtaaacaagt ttcccatttc cctttcttag attgcaccag tgaaggagat gttttgcaaa |
| 4861 | gattcagaga actaattttt cactggataa gacctgagta acccagaccc cccaccgtgg |
| 4921 | ttcttttcac agccctcgac tttgcactta aaaagggata ttgtaaatga aaggctgcag |
| 4981 | tgccagtttt aagaaagaat ttctgtgaag tgtgaggact ctggagtcta gctcacataa |
| 5041 | agagagtgtt atataaaaat ccgacagctg aactaggttg ctcttttttg gcagggagtg |
| 5101 | gggatgagat ttgacaccaa tatgggcaaa attagataac cttttggtta atataaatga |
| 5161 | ttttgatttg gaggcctaat ttgtagattg tgaaagcagc ttttagttta acttattcac |
| 5221 | agacccctta taattaccat gttttttttt ttcttcctaa atctcttggt tcagcttgtg |
| 5281 | aatcttacgt gcccgtaaag ttgggatgtt gaattggctc ttctttgttc tggcagtgag |
| 5341 | tcaagtgtcc agcatttttt cataagtgtt ttttaaaatt gttctccagc attttatggc |
| 5401 | tcctccctcc catgtcctca gacccagcaa aagcgtagag gcagaattag aggcctctcc |
| 5461 | aggccagctc ctctgcccac atgtcataca aggtgtgaat ttgagcacag tccagaaatg |
| 5521 | gagacatccc acccccagtt gaataatggc ccattcatgc caaccttgcc aacacggaga |
| 5581 | gggcagagat gcactagaag accttcatcc tccccttcct ctgccccaag tcactacagt |
| 5641 | tggttctatt gaagccagtc tttaagaaac ctgggttaaa gacaccagca cttctgcttg |
| 5701 | ctgggctggc tggacctgtg aagccatggg caggtagtgc cctcttgaga gtcattttat |
| 5761 | ttggccacct tcaggtgaga ctatccatag acacatgcta ggataggccc cgctgggagg |
| 5821 | gcagttacag gagagagtag gtggtggtga cgtgagggct gtgaaggatc cagagacaag |
| 5881 | acttagatgt ttcgttcatt cactcactca ttcagttact cctaagactt ttcagtttca |
| 5941 | taaggaagag tgttgcctga ggccctaggg aatattgggg aatagaaggg attgaggaaa |
| 6001 | cattaataat agttattcaa aagacccaaa tgcttatact tctctctccc ttcttctctc |
| 6061 | tctgacacac acacacacac acacacacac acacacacac acgtgcacat tcctccctta |
| 6121 | catgctcatt tgtgccttaa atgtgcctta taggtaaatc caggatgact gaggaatccc |
| 6181 | tcgtcactgg gagattttgt atatattctt ttattattag attgagttgg gtgtggggaa |
| 6241 | aaattttttt ctgaaggctc aaaagtggtt tcctaaaagt gagccactat cagatttgca |
| 6301 | catcaggaga aaagaaatag ggttacgtcc attaggaaaa tcccagtttg caggagtgca |
| 6361 | atcacatcaa aaaaacaacc agccaggatt aaaggtatta taaatcctca tagcggaaca |
| 6421 | tttctcaggg caaaggaacc tggctcattt gaagattaat gttccatgcc tttgtggtca |
| 6481 | aagggtcagc acttaacaca ggaaaaaact aggtgttgtt ttgttttgtt attttggaca |
| 6541 | acataaaatt caggaatgtt ttatttagcc ttggtttcta gaaggaaggg aaataatatt |
| 6601 | tcttgagcat ttactagggt gttgcgtgct gtgctaagta aattttaagt ctttcagttt |
| 6661 | tatagatacg gaaaacaagg gtgactcttt accacaggat gaataaagaa ctaagtaata |
| 6721 | tgggaaatgc agcaatttct ggactagctg agccgattcc ttcctgtgag cacactgtaa |
| 6781 | gctttcaagt tctctgggca ggaattacag cacctgtccc ctgcaatggc cctgctgtgt |
| 6841 | gatgctcatc gcttcccttc gtgctggagc agtcccccag gtgtccatct cctatctttt |
| 6901 | tgttccaatc ttctgtgagt tccagctagc aggctttaca tctggggaaa ggaaaaccag |
| 6961 | gggttttagc tctgttctct gctcccatcc ttcgctcacc agctgagtga gaacatgaac |
| 7021 | tttttgcacc atgtacccat ggcttacact acttagaaaa tcaccttttc agataaaaca |
| 7081 | gtttatgagt tcatagagaa caccagcact ctttgacaaa actgtgagtg acccttttta |
| 7141 | aacaatgctg agcaggccct gagctataat caacggtgag ctttaatgtc tatgctgaca |
| 7201 | gttaggtttt gctctctttt gtaacaggtt acgtagacca gcagtgttta aatctaaata |
| 7261 | cgttgtgagt ctgttatctg tcctatcgcg ttttttaaat gactttttat tctttatcat |
| 7321 | agctaagtaa ataccaaaaa aaaaaaaaag ctttgtagga cacttgtact tagtttggga |
| 7381 | aaaaaaaata aattgaaatt gttatgcttt tgtatttcca tttcttgcaa ataaatattt |
| 7441 | tttcttaaat agtaa |

SEQ ID NO: 2 - the amino acid sequence of human Creb312, (RefSeq ID
No: NP_919047, GeneID No: 64764). Bold indicates the transmembrane
domain of Creb312.
MEVLESGEQGVLQWDRKLSELSEPGDGEALMYHTHFSELLDEFS
QNVLGQLLNDPFLSEKSVSMEVEPSPTSPAPLIQAEHSYSLCEEPRAQSPFTHITTSD
SFNDDEVESEKWYLSTDFPSTSIKTEPVTDEPPPGLVPSVTLTITAISTPLEKEEPPL
EMNIGVDSSCQTIIPKIKLEPHEVDQFLNFSPKEAPVDHLHLPPTPPSSHGSDSEGSL
SPNPRLHPFSLPQTHSPSRAAPRAPSALSSSPLLTAPHKLQGSGPLVLTEEEKRTLIA
EGYPIPTKLPLSKSEEKALKKIRRKIKNKISAQESRRKKKEYMDSLEKKVESCSTENL
ELRKKVEVLENTNRTLLQQLQKLQTLVMGKVSRTCKLAGTQTGTCLMVVVLCFAVAFG
SFFQGYGPYPSATKMALPSQHSLQEPYTASVVRSRNLLIYEEHSPPEESSSPGSAGEL
GGWDRGSSLLRVSGLESRPDVDLPHFIISNETSLEKSVLLELQQHLVSAKLEGNETLK
VVELDRRVNTTF
SEQ ID NO: 3 - the amino acid sequence of human nuclear form of Creb312,
(amino acid sequences 1-376 of SEQ ID NO: 2). The nuclear form of Creb312
is also referred to herein as “Creb312(N)” or “Creb312(N)” or
“Creb312(ΔTM)”
MEVLESGEQGVLQWDRKLSELSEPGDGEALMYHTHFSELLDEFS
QNVLGQLLNDPFLSEKSVSMEVEPSPTSPAPLIQAEHSYSLCEEPRAQSPFTHITTSD
SFNDDEVESEKWYLSTDFPSTSIKTEPVTDEPPPGLVPSVTLTITAISTPLEKEEPPL
EMNTGVDSSCQTIIPKIKLEPHEVDQFLNFSPKEAPVDHLHLPPTPPSSHGSDSEGSL
SPNPRLHPFSLPQTHSPSRAAPRAPSALSSSPLLTAPHKLQGSGPLVLTEEEKRTLIA
EGYPIPTKLPLSKSEEKALKKIRRKIKNKISAQESRRKKKEYMDSLEKKVESCSTENL
ELRKKVEVLENINRTLLQQLQKLQTLVMGKVSRTCKLAGTQT
SEQ ID NO: 4:
RNAi #1: TAAGTGTCTGATGAGGAG
SEQ ID NO: 5:
RNAi #2: TTAGGAATAAGTGTCTGGCAT
SEQ ID NO: 6:
RNAi #3: TAGATTAGCAGGTTCCTGGAT
SEQ ID NO: 7 - the amino acid sequence of human intergrin-linked
kinase (ILK) protein (Accession No: NP_004508)
MDDIFTQCREGNAVAVRLWLDNTENDLNQSDDHGFSPLHWACRE
GRSAVVEMLIMRGARINVMNRGDDTPLHLAASHGHRDIVQKLLQYKADINAVNEHGNV
PLHYACFWGQDQVAEDLVANGALVSICNKYSEMPVDKAKAPLRELLRERAEKMGQNLN
RIPYKDTFWKGTTRTRPRNGTLNKHSGIDFKQLNFLTKLNENHSGELWKGRWQGNDIV
VKVLKVRDWSTRKSRDFNEECPRLRIFSHPNVLPVLGACQSPPAPHPTLITHWMPYGS
LYNVLHEGTNFVVDQSQAVKFALDMARGMAFLHTLEPLIPRHALNSRSVMIDEDMTAR
ISMADVKFSFQCPGRMYAPAWVAPEALQKKPEDTNRRSADMWSFAVLLWELVTREVPF
ADLSNMEIGMKVALEGLRPTIPPGISPHVCKLMKICMNEDPAKRPKFDMIVPILEKMQ
DK

1. A method for increasing the survival of a cell in response to stress, comprising introducing into a cell a nucleic acid encoding a nuclear form of Creb312 polypeptide, wherein the nuclear form of Creb312 polypeptide comprises the amino acid residues 1 to 376 of SEQ ID NO: 2 or a biologically active fragment or variant thereof, and wherein the expression of nuclear form of Creb312 polypeptide in the cell increases the survival of the cell in response to stress.

2. A method for inducing the proliferation of a cardiomyocyte, comprising introducing into the cardiomyocyte a nucleic acid encoding a nuclear form of Creb312 polypeptide, wherein the nuclear form of Creb312 polypeptide comprises the amino acid residues 1 to 376 of SEQ ID NO: 2 or a biologically active fragment or variant thereof, and wherein the expression of nuclear form of Creb312 polypeptide in the cardiomyocyte increases the proliferation of the cardiomyocyte.

3. The method of claim 1, wherein the agent comprises a polypeptide of amino acid sequence SEQ ID NO: 3 or a biologically active variant or biologically active fragment thereof.

4. The method of claim 2, wherein the agent comprises a polypeptide of amino acid sequence SEQ ID NO: 3 or a biologically active variant or biologically active fragment thereof.

5. The method of claim 1, wherein the stress is selected from the group consisting of; hormonal stress, mechanical stress, stretch stress, hypoxic stress.

6. The method of claim 1, wherein the cell is a cardiac cell.

7. The method of claim 6, wherein the cardiac cell is selected from the group consisting of; a cardiac progenitor cell, a Isl1+ primordial cardiac progenitor, a cardiomyocyte, a cardiomyocyte precursor.

8. The method of claim 2, wherein the cardiomyocyte is a human cardiomyocyte.

9. The method of claim 1, wherein the cell is a human cell.

10. The method of claim 1, wherein the cardiac cell is present in a subject present in a subject.

11. The method of claim 2, wherein the cardiomyocyte is present in a subject present in a subject.

12. A composition comprising a Creb312(N) polypeptide or a biologically active variant or fragment thereof, or a pharmaceutically acceptable derivative thereof for use in the manufacturer of a medicament to increase the survival of a cell to mechanical stress or the manufacturer of a medicament for treatment of a cardiovascular condition, disease or injury to induce the proliferation of cardiomyocytes.

13. The composition of claim 12, wherein the Creb312(N) polypeptide comprises amino acid residues 1 to 376 of SEQ ID NO: 2 or a biologically active fragment or variant thereof.

14. A method to promote the survival of a transplanted cell to mechanical stress, comprising; introducing into a transplanted cell a nucleic acid encoding a nuclear form of Creb312 polypeptide which is operatively linked to a promoter, wherein the nuclear form of Creb312 polypeptide comprises the amino acid residues 1 to 376 of SEQ ID NO: 2 or a biologically active fragment or variant thereof; wherein expression of a nuclear form of Creb312 polypeptide increases the survival of the transplanted cell to mechanical stress as compared to a cell not expressing Creb312(N) protein.

15. The method of claim 14, wherein the cell is a cardiac progenitor cell.

16. The method of claim 14, further comprising a transplanting the cell into a subject.

17. The method of claim 14, wherein the promoter is a cardiac promoter.

18. The method of claim 14, wherein the promoter is a mechanical stress promoter.

19. The method of claim 14, wherein the cell is a human Isl1+ cardiac progenitor, or a progeny thereof.

20. The method of claim 16, wherein the subject is a human subject.


SEQUENCE LISTING (19 sequences)

SEQ ID NO: 1 - DNA, 7455 bp, Homo sapiens
ctgggtcctggagcagagccgaggagccctggggtccctcaaagtttgtgtctggagccgtagcggcaagtgggcttgcggctaagggattttcctgggatgagagcgggtcttctgccttcattttggatgcacatcccgctttagccccggcagcctttggtccggctcgtgtccctggggattctcggatctccgaggacaccggacgggagcgcttggccatcctctctccggcagaggagcagacgtttgctttccaagtgcaaaactacagacacgcgcgcgcacacacgcaagcacacgcggagagagaggaaccttgccggtccgaggcagctctgcgcgtcccctcctgcgcttagcatcctcggcccagcgcggcccgcaccgccatggaggtgctggagagcggggagcagggcgtgctgcagtgggaccgcaagctgagcgagctgtcagagcccggggacggcgaggccctcatgtaccacacgcacttctcagaacttctggatgagttttcccagaacgtcttgggtcagctcctgaatgatcctttcctctcagagaagagtgtgtcaatggaggtggaaccttccccgacgtccccggcgcctctcatccaggctgagcacagctactccctgtgcgaggagcctcgggcccagtcgcccttcacccacattaccaccagtgacagcttcaatgacgatgaggtggaaagtgagaaatggtacctgtctacagacttcccttcaacatccatcaagacagagccagttacagacgaaccacccccaggactcgttccgtctgtcactctgaccatcacagccatctccaccccgttggaaaaggaggaacctcctctggaaatgaacactggggttgattcctcgtgccagaccattattcctaaaattaagctggagcctcatgaagtggatcagtttctaaacttctctcctaaagaagccccagtggaccacctgcatttgccgcccacccctccgagcagtcacggcagtgactcagagggcagcctgagtcccaacccacgcctgcaccccttcagcctgcctcagacccacagcccctccagagctgcaccccgggccccctccgccctctccagctcccctctcctcacggctcctcataaactgcagggatcaggccctctggtcctgacagaggaggagaagaggaccctgatcgctgagggctatcccatccccaccaaattgcccctgtcaaaatcagaggagaaggccctgaagaaaattcggaggaagatcaagaataagatttctgctcaggaaagtaggagaaagaagaaagaatacatggacagcctggagaaaaaagtggagtcttgttcaactgagaacttggagcttcggaagaaggtagaggttctagagaacactaataggactctccttcagcaactccagaagcttcagactttggtgatgggcaaggtttctcgaacctgcaagttagctggcacgcagactggcacctgcctcatggttgtggtgctgtgctttgccgttgcattcggcagcttctttcaaggctacgggccctatccttctgccaccaagatggctctgcccagccagcattccctgcaggagccctacacagcctccgtggtgagatccagaaacctgctgatctacgaggaacattctcccccagaggagtcatccagcccgggctcggctggggagctggggggctgggatagaggttcctccctgctcagggtgtcagggctggagtccaggccggatgtggatcttccccatttcattatctcgaatgagaccagcctggagaagtcagtgcttttggagctgcagcagcacctggtcagcgccaaactggaggggaatgaaacactaaaagttgtagaactcgacagaagagtgaacaccactttctaaagaggctgcctgcaccccctccctttcccttaactctacttttacatccccaaaccacctttgtcatcagcttttcctctttgccactggatcttcatggagacatgggcaagcattagtggcttcagattggagaccagcctgggacttccctgcagtgagagagcatctccccctggtccatgcccctcctgtgcagaagggagcctgcatccctcccttcctttctcttactgccataggaaattattttaggggttggaggtgggacaagcaggcttgtttccaccaatagtgccaaaaagatattgcctaatgtgcacctgtgaggtgtaaccccccgctttggagacgagatggctcttgttcagtcaagaccccagactctggccacaaaaatgccataatgcctgttggtatttggcaaagcactgacccgtgtcctccgttgctcgcactggggtctctggtgtgaacacccccgacagcagccctccgcccactctgccccctgggagccctcgctggatcgtctcgtctcctgcagcagcactggcaggcgagggctctcgttcatattctcaggccgcaagtgcaatgcctgaggggatcaggcttttctactccaggcaaacctgccccatcttgtcgcttttaggacctcccacaacctggttccccacacatccatagttctgcctccccagcttctcctccccagttgtaaatagtatttattagcttgccgaggcttcctgctagcaaccacactgaagagatcgatgcctcctttcaagctagccaagttttctgcgagccttcagagctaggagggcaccctaggctctgggatcccgtgtctttccagacaatgttttgtttcctttcctttgttttttcttttaactggaataattaccattgaaaaagaagttcctttgagcatgtatgtgtctgcctctaggatgagctcagagcgagagatgacacaatgcctcactcaggccccgggctccctggccacaagctttttctatcctgttttcatgacagagaaggggaagccctgttctgacaacagacatttcagacaaccttgctggctttccacacctgcctggccccctcctccctccacacttccactttgtcctcctcgtcccctacctcaacaaagcagggtggggtaggtgacatttgtgtatccacattcttacctttggtagtcaggtttggctactttgcagctcgcccaaagagatacaacctaatccccaacctacttttagtttttttgttttttttttatggttaaaagtaacttttgtagtttaaaaaaatctttcctctttcatataaataagaagtggaaattgcctttttattgtgtaatgtagaaaaccctcaagtgttttttccgagcttgggaaagattttgtgtaggaaatgtgcatagagtttgtattttatttttattagcagctgaaatgcctttggttttggcttctctctctccctctctctctctgtctctccttctctctctccccccaccacccacccccacacacgtcatctgcattgttattggagcctgtacttagagggattaagcccacaccctggcttccattccatatcaggtacaggatttgatgttattaacatttgtcgtcatacctcataagtcggtccctgccttgtctgtctaggcccatttggggctccctgtgagtgattcccctctctctgctatgctggagacggttccagcctggaaagcggccaagttcatcttctcactgtgagtggaagctggatcgggcccccgtagtcctggcagccctgttgtctggagggttcttgttgtccctcccattagccagggcggagactgtctgagctgtgcaggaggagggttgctagtaggttctgcttctgcttctctctgctccactgtctgcagcccagatcctgttgggcctggctggtgtctggtaaccatgggcctccactgacccatccctctcttttaaactgtcaggtcattatcaggcataggcagcctatagggcccaaagaaggcaaaaagataagatttactcaagtagcatttgggcaatgaggaaggaaaggtttcaaatttaggggcagaagtgagagaatgagccaacccatgtacctgctgcaactgaaccagactgggttttcaaggctcccagacgtagagtaggaaacgtgctcttctaaatgaggagggagaagataaaggaaacttctagcccctgtccttagtgctttgaggattttattttctcccttactacgcttgcttgacgtcactctctctcgacctccaaacagcaggactctttctctgggaaaccatccttccaaaacggaatctatgtagacaatgggacgttaggcagagagctcagatggcccttttaagggggctccaagaaccaacatcactgctcttttagataaacctctgccctccactccttgcttgagtgggttaaaggaactaacagttgtccctttaggaggacaaaatggggtcaagaggacacagaagagttgtatagcaccagattggttccaaatagttaatggatgtgtgcacattttctgttcagggattaagaccagaatatcagtggatttgttttccccaccaagtggcctcttagactagtcattaacttatgattagctctaaagatttcaaatagtggcagacagtgtcttctgaatgtaagttttgagaaatacgagtctgtcagagcggccataagccataaagagtcaatctcttaattatatttttcatcatgtaaacaagtttcccatttccctttcttagattgcaccagtgaaggagatgttttgcaaagattcagagaactaatttttcactggataagacctgagtaacccagaccccccaccgtggttcttttcacagccctcgactttgcacttaaaaagggatattgtaaatgaaaggctgcagtgccagttttaagaaagaatttctgtgaagtgtgaggactctggagtctagctcacataaagagagtgttatataaaaatccgacagctgaactaggttgctcttttttggcagggagtggggatgagatttgacaccaatatgggcaaaattagataaccttttggttaatataaatgattttgatttggaggcctaatttgtagattgtgaaagcagcttttagtttaacttattcacagaccccttataattaccatgtttttttttttcttcctaaatctcttggttcagcttgtgaatcttacgtgcccgtaaagttgggatgttgaattggctcttctttgttctggcagtgagtcaagtgtccagcattttttcataagtgttttttaaaattgttctccagcattttatggctcctccctcccatgtcctcagacccagcaaaagcgtagaggcagaattagaggcctctccaggccagctcctctgcccacatgtcatacaaggtgtgaatttgagcacagtccagaaatggagacatcccacccccagttgaataatggcccattcatgccaaccttgccaacacggagagggcagagatgcactagaagaccttcatcctccccttcctctgccccaagtcactacagttggttctattgaagccagtctttaagaaacctgggttaaagacaccagcacttctgcttgctgggctggctggacctgtgaagccatgggcaggtagtgccctcttgagagtcattttatttggccaccttcaggtgagactatccatagacacatgctaggataggccccgctgggagggcagttacaggagagagtaggtggtggtgacgtgagggctgtgaaggatccagagacaagacttagatgtttcgttcattcactcactcattcagttactcctaagacttttcagtttcataaggaagagtgttgcctgaggccctagggaatattggggaatagaagggattgaggaaacattaataatagttattcaaaagacccaaatgcttatacttctctctcccttcttctctctctgacacacacacacacacacacacacacacacacacacacgtgcacattcctcccttacatgctcatttgtgccttaaatgtgccttataggtaaatccaggatgactgaggaatccctcgtcactgggagattttgtatatattcttttattattagattgagttgggtgtggggaaaaatttttttctgaaggctcaaaagtggtttcctaaaagtgagccactatcagatttgcacatcaggagaaaagaaatagggttacgtccattaggaaaatcccagtttgcaggagtgcaatcacatcaaaaaaacaaccagccaggattaaaggtattataaatcctcatagcggaacatttctcagggcaaaggaacctggctcatttgaagattaatgttccatgcctttgtggtcaaagggtcagcacttaacacaggaaaaaactaggtgttgttttgttttgttattttggacaacataaaattcaggaatgttttatttagccttggtttctagaaggaagggaaataatatttcttgagcatttactagggtgttgcgtgctgtgctaagtaaattttaagtctttcagttttatagatacggaaaacaagggtgactctttaccacaggatgaataaagaactaagtaatatgggaaatgcagcaatttctggactagctgagccgattccttcctgtgagcacactgtaagctttcaagttctctgggcaggaattacagcacctgtcccctgcaatggccctgctgtgtgatgctcatcgcttcccttcgtgctggagcagtcccccaggtgtccatctcctatctttttgttccaatcttctgtgagttccagctagcaggctttacatctggggaaaggaaaaccaggggttttagctctgttctctgctcccatccttcgctcaccagctgagtgagaacatgaactttttgcaccatgtacccatggcttacactacttagaaaatcaccttttcagataaaacagtttatgagttcatagagaacaccagcactctttgacaaaactgtgagtgaccctttttaaacaatgctgagcaggccctgagctataatcaacggtgagctttaatgtctatgctgacagttaggttttgctctcttttgtaacaggttacgtagaccagcagtgtttaaatctaaatacgttgtgagtctgttatctgtcctatcgcgttttttaaatgactttttattctttatcatagctaagtaaataccaaaaaaaaaaaaaagctttgtaggacacttgtacttagtttgggaaaaaaaaataaattgaaattgttatgcttttgtatttccatttcttgcaaataaatattttttcttaaatagtaa

SEQ ID NO: 2 - protein, 520 aa, Homo sapiens
MEVLESGEQGVLQWDRKLSELSEPGDGEALMYHTHFSELLDEFSQNVLGQLLNDPFLSEKSVSMEVEPSPTSPAPLIQAEHSYSLCEEPRAQSPFTHITTSDSFNDDEVESEKWYLSTDFPSTSIKTEPVTDEPPPGLVPSVTLTITAISTPLEKEEPPLEMNTGVDSSCQTIIPKIKLEPHEVDQFLNFSPKEAPVDHLHLPPTPPSSHGSDSEGSLSPNPRLHPFSLPQTHSPSRAAPRAPSALSSSPLLTAPHKLQGSGPLVLTEEEKRTLIAEGYPIPTKLPLSKSEEKALKKIRRKIKNKISAQESRRKKKEYMDSLEKKVESCSTENLELRKKVEVLENTNRTLLQQLQKLQTLVMGKVSRTCKLAGTQTGTCLMVVVLCFAVAFGSFFQGYGPYPSATKMALPSQHSLQEPYTASVVRSRNLLIYEEHSPPEESSSPGSAGELGGWDRGSSLLRVSGLESRPDVDLPHFIISNETSLEKSVLLELQQHLVSAKLEGNETLKVVELDRRVNTTF

SEQ ID NO: 3 - protein, 376 aa, Homo sapiens
MEVLESGEQGVLQWDRKLSELSEPGDGEALMYHTHFSELLDEFSQNVLGQLLNDPFLSEKSVSMEVEPSPTSPAPLIQAEHSYSLCEEPRAQSPFTHITTSDSFNDDEVESEKWYLSTDFPSTSIKTEPVTDEPPPGLVPSVTLTITAISTPLEKEEPPLEMNTGVDSSCQTIIPKIKLEPHEVDQFLNFSPKEAPVDHLHLPPTPPSSHGSDSEGSLSPNPRLHPFSLPQTHSPSRAAPRAPSALSSSPLLTAPHKLQGSGPLVLTEEEKRTLIAEGYPIPTKLPLSKSEEKALKKIRRKIKNKISAQESRRKKKEYMDSLEKKVESCSTENLELRKKVEVLENTNRTLLQQLQKLQTLVMGKVSRTCKLAGTQT

SEQ ID NO: 4 - DNA, 19 bp, Artificial Sequence
  Description of Artificial Sequence Synthetic oligonucleotide
ataagtgtctgatgaggag

SEQ ID NO: 5 - DNA, 21 bp, Artificial Sequence
  Description of Artificial Sequence Synthetic oligonucleotide
ttaggaataagtgtctggcat

SEQ ID NO: 6 - DNA, 21 bp, Artificial Sequence
  Description of Artificial Sequence Synthetic oligonucleotide
tagattagcaggttcctggat

SEQ ID NO: 7 - protein, 452 aa, Homo sapiens
MDDIFTQCREGNAVAVRLWLDNTENDLNQGDDHGFSPLHWACREGRSAVVEMLIMRGARINVMNRGDDTPLHLAASHGHRDIVQKLLQYKADINAVNEHGNVPLHYACFWGQDQVAEDLVANGALVSICNKYGEMPVDKAKAPLRELLRERAEKMGQNLNRIPYKDTFWKGTTRTRPRNGTLNKHSGIDFKQLNFLTKLNENHSGELWKGRWQGNDIVVKVLKVRDWSTRKSRDFNEECPRLRIFSHPNVLPVLGACQSPPAPHPTLITHWMPYGSLYNVLHEGTNFVVDQSQAVKFALDMARGMAFLHTLEPLIPRHALNSRSVMIDEDMTARISMADVKFSFQCPGRMYAPAWVAPEALQKKPEDTNRRSADMWSFAVLLWELVTREVPFADLSNMEIGMKVALEGLRPTIPPGISPHVCKLMKICMNEDPAKRPKFDMIVPILEKMQDK

SEQ ID NO: 8 - DNA, 13 bp, Artificial Sequence
  Description of Artificial Sequence Synthetic oligonucleotide
tcctgagctcagc

SEQ ID NO: 9 - protein, 70 aa, Unknown
  Description of Unknown Oasis peptide
SKISRPYKMAATQTGTCLMVAALCFVLVLGSLVPCLPAFSSGSMTVKEDPIAADSVYAASQMPSRSLLFY

SEQ ID NO: 10 - protein, 70 aa, Unknown
  Description of Unknown Creb3l2 peptide
GKVSRTCKLAGTQTGTCLMVVVLCFAVAFGSFFQGYGPYPSATKMALPSQHPLSEPYTASVVRSRNLLIY

SEQ ID NO: 11 - protein, 70 aa, Unknown
  Description of Unknown ATF6 peptide
SENQRLKVPSPKRRAVCVMIVLAFIMLNYGPMSMLEQESRRVKPSVSPANQRRHLLEFSAKEVKDTSDGD

SEQ ID NO: 12 - protein, 70 aa, Unknown
  Description of Unknown Srebp2 peptide
EPDSPPVALGMVDRSRILLCVLTFLGLSFNPLTSLLQWGGAHNTDQHPYSGSGRSVLSLESGAGGWFDWM

SEQ ID NO: 13 - DNA, 16 bp, Artificial Sequence
  Description of Artificial Sequence Synthetic oligonucleotide
gtcctgagctcagcag

SEQ ID NO: 14 - DNA, 16 bp, Artificial Sequence
  Description of Artificial Sequence Synthetic oligonucleotide
gtccctatctcagcag

SEQ ID NO: 15 - DNA, 16 bp, Artificial Sequence
  Description of Artificial Sequence Synthetic oligonucleotide
gtcctgagcctatcag

SEQ ID NO: 16 - DNA, 16 bp, Artificial Sequence
  Description of Artificial Sequence Synthetic oligonucleotide
gtcctgagctcgttcg

SEQ ID NO: 17 - DNA, 19 bp, Artificial Sequence
  Description of Artificial Sequence Synthetic oligonucleotide
gtcctgagctcagccttaa

SEQ ID NO: 18 - protein, 5 aa, Artificial Sequence
  Description of Artificial Sequence Synthetic peptide
GGGGC

SEQ ID NO: 19 - DNA, 23 bp, Artificial Sequence
  Description of Artificial Sequence Synthetic oligonucleotide
aannnnnnnnnnnnnnnnnnntt